(19) United States
(12) Patent Application Publication
(10) Pub. No.: US 2019/0218304 A1
(43) Pub. Date: Jul. 18, 2019

(54) POTENTIATING THE EFFECT OF ATP RELEASE

(71) Applicant: INNATE PHARMA, Marseille (FR)

(72) Inventors: Stéphanie CHANTEUX, Marseille (FR); Nicolas GOURDIN, Marseille (FR); Carine PATUREL, Marcy l'Etoile (FR); Ivan PERROT, Cassis (FR); Benjamin ROSSI, Marseille (FR)

(21) Appl. No.: 16/370,789

(22) Filed: Mar. 29, 2019

Related U.S. Application Data
(63) Continuation-in-part of application No. PCT/EP2018/081364, filed on Nov. 15, 2018.
(60) Provisional application No. 62/586,224, filed on Nov. 15, 2017.
(60) Provisional application No. 62/686,149, filed on Jun. 18, 2018.
(60) Provisional application No. 62/733,175, filed on Sep. 19, 2018.

(51) Int. Cl. C07K 16/28; A61P 35/00; C07K 16/40; A61K 39/395; A61K 31/282

(52) U.S. Cl. CPC C07K 16/2896; A61P 35/00; C07K 16/2818; A61K 2039/505; A61K 39/3955; A61K 31/282; C07K 2317/76; C07K 16/40

(57) ABSTRACT

The present invention relates to methods of using compounds that inhibit the enzymatic activity of soluble human CD39 in the treatment of cancer to potentiate the activity of an agent or treatment that induces the extracellular release of ATP from tumor cells and/or induces the death of tumor cells.

### CROSS-REFERENCE TO RELATED APPLICATIONS

This application claims the benefit of U.S. Provisional Application Nos. 62/586,224 filed 15 Nov. 2017, U.S. 62/686,149 filed 18 Jun. 2018, U.S. 62/733,175 filed 18 Sep. 2018 and International patent application no. PCT/EP2018/081364 filed 15 Nov. 2018; all of which are incorporated herein by reference in their entireties; including any drawings.

### REFERENCE TO SEQUENCE LISTING

The present application is being filed along with a Sequence Listing in electronic format. The Sequence Listing is provided as a file entitled “CD39-8_CIP_ST25”, created 28 Mar. 2019, which is 85 KB in size. The information in the electronic format of the Sequence Listing is incorporated herein by reference in its entirety.

### FIELD OF THE INVENTION

This invention relates to the use of CD39 neutralizing agents for the treatment of cancer.

### BACKGROUND OF THE INVENTION

NTPDase 1 (ectonucleoside triphosphate diphosphohydrolasel), also known as CD39/ENTPD1 or vascular CD39, functions together with another enzyme, CD73 (ecto-5′-nucleotidase), to hydrolyze extracellular adenosine triphosphate (ATP) and adenosine diphosphate (ADP) to generate adenosine, which binds to adenosine receptors and inhibits T-cell and natural killer (NK)-cell responses, thereby suppressing the immune system. The generation of adenosine via the CD73/CD39 pathway is recognized as a major mechanism of regulatory T cell (Treg) immunosuppressive function. CD39 has two transmembrane domains near the N- and C-terminal ends, short cytoplasmic N- and C-terminal segments, and a large extracellular domain containing the active site. However, while CD39 is typically anchored to the membrane by the two transmembrane domains at the two ends of the molecule, it has recently also been reported that a soluble catalytically active form of CD39 can be found in circulation in human and mice (Yegutkin et al., (2012) FASEB J. 26(9): 3875-3883).

Radiotherapy and some chemotherapeutic agents have been shown induce specific immune responses resulting in immunogenic cancer cell death (Martins et al. 2009 Cell Cycle 8(22): 3723-3728). An anti-tumor immune response induced by such treatments depends on the capacity of dendritic cells (DC) to present antigen from dying cancer cells and to prime tumor-specific cytotoxic T lymphocytes (CTL). To mount a CTL response, DC must incorporate antigens from stressed or dying cells, acquire the competence of antigen processing in a maturation step and present antigenic peptides bound to MHC molecules in the context of costimulatory signals and cytokines that stimulate the differentiation/activation of specific CTL.

There remains, however, a need to improve the efficacy of current therapies designed to eliminate cancer cells, including radiotherapy and chemotherapeutic agents.

### SUMMARY OF THE INVENTION

The present invention arises, inter alia, from the discovery that antibodies that neutralize the ATPase activity of the CD39 protein in the presence of significant concentrations of ATP are able to reverse the immunosuppressive effect of CD39 in dendritic cells (DCs) in the presence of exogenously added ATP. The antibodies are furthermore able to induce or increase the proliferation of T cells co-cultured with the DCs. The ability to reduce the CD39-mediated inhibition of DC activation provides an advantageous use of antibodies in combination with agents or treatments that induce the extracellular release of ATP from tumor cells, notably agents or treatments that induce immunogenic cancer cell death, e.g., agents or treatments that induce the death of tumor cells (inter alia, chemotherapeutic agents, radiotherapy). While the ATP release has the potential to be immunogenic and promote DC activation, it can also be subject to catabolism by CD39, in turn suppressing the immunogenic effect of the extracellular ATP. Furthermore, during chemotherapy increased levels of ATP are associated with higher expression of CD39 on DCs. Within an ATP-enriched tumor microenvironment, infiltrating DCs may contribute to ATP degradation by modulating CD39 expression, in turn decreasing the chemotherapy-induced immunogenic tumor cell death. Combined use with the anti-CD39 antibodies thus permits the potentiation of the immunogenic effect of agents or treatments that induce the death (e.g., apoptosis or necrosis) of tumor cells.

Accordingly, in one aspect the present invention provides improved methods of enhancing an anti-tumor immune response, via the use of antibodies that bind and neutralize CD39 in the presence of ATP, in combination with an agent or treatment that induces the death of tumor cells, e.g., an agent or treatment that is capable of inducing the extracellular release of ATP from tumor cells, an agent or treatment that induces immunogenic cancer cell death. In one aspect the present invention provides improved methods of enhancing an anti-tumor immune response, via the use of antibodies that are capable of binding and neutralizing CD39 in the presence of ATP, in combination with a means for inducing the death of tumor cells, e.g., a means for inducing apoptosis and/or the extracellular release of ATP from tumor cells. In one aspect provided is, in a method of treating a tumor in a human individual by administering an agent or treatment that induces the extracellular release of ATP, the improvement comprising further administering to the individual an effective amount of an antibody that is capable of binding and inhibiting the ATPase activity of a soluble extracellular domain human CD39 in the presence of exogenously added ATP. In one aspect provided is, in a method of treating a tumor in a human individual by administering an antibody that is capable of binding and inhibiting the ATPase activity of a soluble extracellular domain human CD39 in the presence of exogenously added ATP, the improvement comprising further administering to the individual an effective amount of an agent or treatment that induces the extracellular release of ATP. In one aspect, provided is a method of treating a tumor in a human individual, the treatment comprising administering to the individual an effective amount of each of: (a) a means for inhibiting the ATPase activity of a soluble extracellular domain human CD39, and (b) a means for inducing the extracellular release of ATP from tumor cells. In one aspect, provided is, in a method of treating a tumor in a human individual by administering an agent or treatment that induces the extracellular release of ATP, the improvement comprising further administering to the individual an effective amount of means for inhibiting the ATPase activity of a soluble extracellular domain human CD39. In one aspect of any embodiment herein, the agent or treatment (or means) that is capable of inducing the extracellular release of ATP from tumor cells comprises an anthracycline, an oxaliplatin, a cisplatin, X-rays, a PARP inhibitor, a taxane, an anthracycline, a DNA damaging agent, a camptothecin, an epothilone, a mytomycin, a combretastatin, a vinca alkaloid, a nitrogen mustard, a maytansinoid, a calicheamycin, a duocarmycin, a tubulysin, a dolastatin, an auristatin, an enediyne, an amatoxin, a pyrrolobenzodiazepine, an ethylenimine, a radioisotope, a therapeutic protein or peptide toxin, or an antibody that binds an antigen expressed by a tumor cell and that mediates ADCC. In one embodiment, the antibody that is capable of binding and neutralizing CD39 is capable of neutralizing the activity of both soluble extracellular domain CD39 protein (sCD39) and membrane-bound CD39 protein (memCD39). As shown herein, the antibodies that are able to reverse the immunosuppressive effect of CD39 in dendritic cells (DCs) in the presence of exogenously added ATP are also characterized by being capable of neutralizing the activity of both soluble extracellular domain CD39 protein (sCD39) and membrane-bound CD39 protein (memCD39). Notably, antibody BY40 which is unable to reverse the immunosuppressive effect of CD39 in dendritic cells (DCs) in the presence of exogenously ATP is also unable to neutralize the ATPase activity of soluble extracellular domain CD39 protein (sCD39) and has a lower maximal inhibition of membrane-bound CD39 protein (memCD39) ATPase activity.

Without wishing to be bound by theory, it is believed that antibodies that neutralize membrane-bound CD39 at the cell surface operate by inhibiting the domain motion of membrane-bound CD39 (memCD39), however without similarly affecting the activity of the soluble CD39 protein (sCD39). It has been reported that memCD39 occurs as homo-multimers (e.g., tetramers and/or other multimers, in addition to monomeric forms) while sCD39 is a monomer, and moreover that the transmembrane domains in memCD39 undergo dynamic motions that underlie a functional relationship with the active site (Schulte am Esch et al. 1999 Biochem. 38(8):2248-58). Antibodies that block only memCD39 may recognize CD39 outside of the enzyme active site and prevent multimerization without blocking the monomeric form of CD39. Blockade of multimerization may reduce enzyme activity, and it has been reported that CD39 multimerization substantially augments ATPase activity. In contrast, antibodies that also block sCD39 may interfere with CD39 substrate and inhibit monomeric form of the enzyme. Such antibodies may also prevent multimerization of memCD39, thus providing a second mechanism of inhibition of the enzymatic activity of CD39. In the presence of ATP (e.g., as in the tumor environment), partial inhibition of CD39 by prevention of multimerization without blockade of sCD39 may lead to sufficient residual AMP to prevent any detectable additive effect on activation of DC. Consequently, antibodies that bind and inhibit the ATPase activity of monomeric and/or soluble CD39 (e.g., monomeric sCD39) can be used advantageously to achieve greater neutralization of CD39 activity in an individual by neutralizing both membrane-bound and soluble CD39 protein (an extracellular domain protein in solution).

In one aspect, provided herein is an agent that binds CD39 and inhibits the enzymatic (ATPase activity) activity of human CD39 protein, for use in the treatment of cancer, wherein the agent that binds CD39 is administered in combination with an agent that induces the extracellular release of ATP from tumor cells, optionally an agent that induces the death of tumor cells, optionally inducing apoptosis and/or necrosis. In another aspect, provided herein is an agent that induces the extracellular release of ATP from tumor cells, optionally an agent that induces the death of tumor cells, optionally inducing apoptosis and/or necrosis, for use in the treatment of cancer, wherein the agent that induces the extracellular release of ATP from tumor cells is administered in combination with an agent that binds CD39 and inhibits the enzymatic (ATPase activity) activity of human CD39 protein.

In one embodiment, provided is a method for treating or preventing a cancer in an individual, the method comprising administering to an individual: (a) an agent that binds and that inhibits the ATPase activity of a CD39 protein, and (b) an agent that is capable of inducing the extracellular release of ATP from tumor cells.

In one embodiment, provided is a method of potentiating the anti-tumor effect of an antibody that is capable of binding and inhibiting the ATPase activity of CD39 in the presence of exogenously added ATP, the method comprising administering to the individual an agent or treatment that induces the extracellular release of ATP from tumor cells and/or induces the death of tumor cells.

In one embodiment, provided is a method of sensitizing an individual to treatment with an agent or treatment that induces the extracellular release of ATP from tumor cells, comprising administering to the individual a means of binding and inhibiting the ATPase activity of a soluble CD39 in the presence of exogenously added ATP (or, e.g., a pharmaceutical composition comprising such means). In one embodiment, provided is a method of sensitizing an individual to treatment with an agent or treatment that induces the extracellular release of ATP from tumor cells, comprising administering to the individual an antibody that is capable of binding and inhibiting the ATPase activity of a soluble CD39 in the presence of exogenously added ATP.

In one embodiment, provided is a method of treating cancer in an individual having a poor response, or prognostic for response, to treatment with an agent or treatment that induces the extracellular release of ATP from tumor cells and/or induces the death of tumor cells (in the absence of combined treatment with anti-CD39 antibody), the method comprising administering to the individual an antibody that is capable of binding and inhibiting the ATPase activity of CD39 in the presence of exogenously added ATP.

In one embodiment, the agent that is capable of inducing the extracellular release of ATP from tumor cells directly induces apoptosis of tumor cells. In one embodiment, the agent that is capable of inducing the extracellular release of ATP from tumor cells directly induces necrosis of tumor cells. In one embodiment, the agent comprises a cytotoxic agent that directly causes the death of tumor cells, optionally a chemotherapeutic agent used in the treatment of cancer. In one embodiment, the agent comprises a depleting antibody. In one embodiment, the agent comprises an immunoconjugate comprising an antibody that specifically binds a protein expressed by a tumor cell and a cytotoxic agent. In one embodiment, the agent comprises an antibody that specifically binds a protein expressed by a tumor cell and that is not conjugated to a cytotoxic agent (e.g., a naked antibody). Optionally the antibody is capable of directly inducing the apoptosis of tumor cells.

In one embodiment, the agent that binds CD39 and inhibits the ATPase activity of human CD39 protein is capable of neutralizing the ATPase activity of CD39 in the presence of exogenously added ATP.

In one embodiment, the agent that binds CD39 and inhibits the ATPase activity of human CD39 protein is capable of neutralizing the ATPase activity of a soluble extracellular domain human CD39 protein. Optionally the agent is capable of neutralizing the ATPase activity of the soluble extracellular domain human CD39 protein in the presence of exogenously added ATP, optionally wherein added ATP at a concentration of 20 μM. Assays can be for example as shown in the Examples herein, e.g., anti-CD39 antibody is incubated in plates with soluble recombinant human CD39 protein for 1 h at 37° C., 20 μM ATP is added to the plates for 30 additional minutes at 37° C. before addition of CTG (Cell Titer Glo) reagent, and emitted light is quantified using an Enspire™ luminometer after a short incubation period of 5 min in the dark.

Optionally, the antibody is capable of causing a decrease in the ATPase activity of the human extracellular domain CD39 protein in solution by more than 50%, optionally more than 60%, 70%, 75% or 80%.

In one embodiment, an agent that binds CD39 will provide an at least 50%, 60%, 70%, 75%, 80% or 90% reduction in the ATPase activity of a soluble human CD39 protein (e.g., as assessed by the methods disclosed herein), optionally further at a concentration compatible with administration of an antibody to a human.

In one embodiment, the agent that binds CD39 and inhibits the ATPase activity of human CD39 protein is capable of causing an increase in expression of a cell surface marker of activation in monocyte-derived dendritic cells, when such moDC are incubated in vitro with the antibody and ATP, optionally wherein exogenously added ATP is provided at 0.125 mM, 0.25 mM or 0.5 mM.

In one embodiment, the agent that binds CD39 and inhibits the ATPase activity of human CD39 protein is capable of binding and neutralizing the ATPase activity of human CD39 at the surface of a cell. In one embodiment, the agent is capable of increasing the activation of dendritic cells in the presence of ATP. In one embodiment, the agent is capable of causing an increase in expression of a cell surface marker of activation in monocyte-derived dendritic cells, when such moDC are incubated in vitro with the antibody and ATP. Optionally, ATP is exogenously added ATP provided at 0.125 mM, 0.25 mM or 0.5 mM. Optionally, an increase in expression of a cell surface marker of activation is assessed by incubating moDC in presence of ATP for 24 hours and analyzing cell surface expression of CD80, CD83 and/or HLA-DR on moDC by flow cytometry. Optionally, the increase in expression of a cell surface marker is at least 40%, 50%, 75% or 80%, compared to a negative control (e.g., medium).

In one aspect of any embodiment herein, the agent that inhibits or neutralizes the ATPase activity of a CD39 protein is or comprises an antibody or antibody fragment that binds CD39 protein (e.g., a monospecific antibody, a bispecific or multispecific antibody).

The combined use of the agents will be useful in promoting an adaptive immune response against a tumor by increasing the pool of available ATP in the tumor microenvironment. These antibodies will therefore be useful in reversing the immunosuppressive effect of CD39 on the activity of DCs and/or T cells. In one embodiment, the methods of the disclosure are useful for increasing or enhancing anti-tumor immunity, for reducing immunosuppression, for enhancing an adaptive anti-tumor immune response (optionally further for enhancing dendritic cell activation and/or dendritic cell mediated T cell proliferation) or for activating and/or potentiating the activity of a DC, a T cell, a tumor-infiltrating and/or tumor-specific T cell, in an individual.

In one aspect of any embodiment herein, the sCD39 protein can be characterized as lacking the two transmembrane domains (i.e. the transmembrane domains near the N- and C-terminal ends) found in membrane bound CD39. In one embodiment, sCD39 is a non-membrane bound sCD39 protein found in circulation, e.g., in a human individual. In one embodiment, sCD39 comprises or consists of the amino acid sequence of SEQ ID NO: 2 (optionally further comprising a C-terminal tag or another non-CD39-derived amino acid sequence), for example a sCD39 protein as produced in the Examples herein. In one embodiment, the protein, antibody or antibody fragment inhibits or neutralizes the ATPase activity of sCD39 when incubated with sCD39 in solution, e.g., according to the methods disclosed herein. In one embodiment, the protein, antibody or antibody fragment specifically binds the human CD39 protein, both in soluble (extracellular domain protein) and in membrane-bound form.

In one aspect of any embodiment herein, the individual can be specified to be a human.

In one embodiment, the anti-CD39 antibody is administered in a therapeutically effective amount.

In one embodiment, the anti-CD39 antibody is administered to an individual having a cancer in an amount and frequency sufficient to neutralize the activity of CD39 (sCD39 and/or memCD39) in the periphery and/or in the tumor microenvironment. In one embodiment, the antibody is administered in an amount and frequency sufficient to decrease the catabolism of ATP in the tumor microenvironment. Optionally, the antibody is administered in an amount and frequency sufficient to provide continued inhibition of the activity of CD39 (sCD39 and/or memCD39) in the periphery and/or in the tumor microenvironment, and/or continued decrease the catabolism of ATP in the tumor microenvironment, for the duration of time between two successive administration of the agent capable of inducing the extracellular release of ATP from tumor cells.

In one embodiment, the agent capable of inducing the extracellular release of ATP from tumor cells is administered in a therapeutically effective amount. In one embodiment, the agent capable of inducing the extracellular release of ATP from tumor cells is administered to an individual having a cancer in an amount and frequency sufficient to induce the death, apoptotis and/or necrosis of tumor cells. In one embodiment, the agent capable of inducing the extracellular release of ATP from tumor cells is administered to an individual having a cancer in an amount and frequency sufficient to induce the extracellular release of ATP in the tumor microenvironment.

In one embodiment, the anti-CD39 antibody and agent capable of inducing the extracellular release of ATP from tumor cells is each administered for at least one administration cycle, the administration cycle comprising at least a first and second (and optionally a 3^{rd}, 4^{th}, 5^{th}, 6^{th}, 7^{th} and/or 8^{th} or further) administration of the anti-CD39 antibody and agent capable of inducing the extracellular release of ATP from tumor cells.

In one embodiment, the cancer is a leukemia, a glioma or glioblastoma, or a cancer of the bladder, breast, colon, esophagus, kidney, liver, lung, ovary, uterus, prostate, pancreas, stomach, cervix, thyroid, head and neck (head and neck squamous cell carcinoma, and skin (e.g., melanoma). In one embodiment the cancer is an advanced and/or refractory solid tumor. In one embodiment the cancer is an advanced and/or refractory solid tumor. In one non-limiting embodiment, the cancer (e.g., the advanced refractory solid tumor) is selected from the group consisting of non-small cell lung cancer (NSCLC), kidney cancer, pancreatic or esophagus adenocarcinoma, breast cancer, renal cell carcinoma (RCC), melanoma, colorectal cancer, and ovarian cancer (and optionally a further cancer type described herein).

In certain optional aspects an anti-CD39 agent can be used to treat a cancer in an individual having a cancer or tumor characterized by immunosuppression, optionally lack of or insufficient immune infiltrate in tumors, optionally lack of or insufficient anti-tumor immunity.

In certain optional aspects the treatments disclosed herein can be used to treat a cancer in an individual having a poor disease prognosis, notably a poor prognosis for response to treatment with an anti-cancer agent, e.g., an agent capable of inducing the extracellular release of ATP from tumor cells, a cytotoxic agent, a chemotherapeutic agent, or an agent that inhibits the enzymatic activity of CD39. An individual having a poor disease prognosis is, for example, at a higher risk of progression, based on one or more predictive factors. In one embodiment, a predictive factor(s) comprises presence or absence of a mutation in one or more genes. In one embodiment, the predictive factor(s) comprises level(s) of expression of one or more genes or proteins, or example inhibitory or activating receptors on immune effector cells. In one embodiment, a predictive factor(s) comprises presence (e.g., numbers) of cells in circulation or in the tumor environment expressing CD39, and/or expression levels of CD39 on the surface of cells in circulation or in the tumor environment; in one embodiment, the cells are tumor cells; in one embodiment the cells are leukocytes, e.g., B cells, regulatory T cells (Treg); in one embodiment the cells are dendritic cells. Presence of elevated expression of CD39, and/or elevated numbers of CD39 expressing cells can indicate an individual has a poor prognosis for response to treatment with an antibody that neutralizes CD39.

In any aspect herein, an individual may be an individual who is a non-responder, or who has experienced a partial or an incomplete response to treatment with an agent capable of inducing the extracellular release of ATP from tumor cells, or whose disease has relapsed or progressed following treatment with an agent capable of inducing the extracellular release of ATP from tumor cells.

In one aspect, provided is an anti-CD39 agent for use in the treatment a cancer in an individual who is a non-responder, or who has experienced a partial or an incomplete response to treatment with an agent capable of inducing the extracellular release of ATP from tumor cells, or whose disease has relapsed or progressed following treatment with an agent capable of inducing the extracellular release of ATP from tumor cells. In one embodiment, the anti-CD39 agent is administered in combination with a treatment (e.g., an agent) capable of inducing the extracellular release of ATP from tumor cells. Optionally, the anti-CD39 agent is capable of binding and inhibiting the ATPase activity of CD39 in the presence of ATP and/or capable of binding and inhibiting the ATPase activity of a soluble extracellular domain human CD39 protein.

In one embodiment, the anti-CD39 agent competes for binding to an epitope or determinant on CD39 with the antibody I-394, I-395, I-396, I-397, I-398, I-399, 31414, 31895, 31873, 31901 or 31905. In one embodiment, the anti-CD39 agent competes for binding to CD39 with an antibody having the heavy and light chains of SEQ ID NOS: 37 and 38 respectively. The agent can be, e.g., a human or humanized anti-CD39 antibody. In one embodiment, the anti-CD39 antibody is an antibody comprising the heavy chain CDRs of the heavy chain of SEQ ID NOS: 37 and the light chain CDRs of the light chain of SEQ ID NO: 37 respectively. In one embodiment, the anti-CD39 antibody comprises a heavy chain comprising an amino acid sequence at least 60%, 70%, 75%, 80%, 85%, 90% or 95% identical to the heavy chain amino acid sequence of SEQ ID NO: 37 and a light chain comprising an amino acid sequence at least 60%, 70%, 75%, 80%, 85%, 90% or 95% identical the light chain amino acid sequence of SEQ ID NO: 38 respectively. In one embodiment, the anti-CD39 agent competes with the antibody having the heavy and light chain variable region amino acid sequences of SEQ ID NOS: 81 and 82 respectively, in binding to an epitope or determinant on CD39. The agent can be, e.g., a human or humanized anti-CD39 antibody. In one embodiment, the anti-CD39 antibody comprises a heavy chain variable region comprising an amino acid sequence at least 60%, 70%, 75%, 80%, 85%, 90 or 95% identical to the heavy chain variable region amino acid sequence of SEQ ID NO: 81 and a light chain variable region comprising an amino acid sequence at least 60%, 70%, 75%, 80%, 85%, 90 or 95% identical the light chain variable region amino acid sequence of SEQ ID NO: 82 respectively.

In certain optional aspects, individuals can be identified for treatment with a CD39-neutralizing agent and an agent capable of inducing the extracellular release of ATP from tumor cells by assessing whether the patient is a poor responder (has a poor prognosis for response) for an anti-cancer agent (e.g., a composition comprising a cytotoxic compound, a chemotherapeutic agent, a depleting antibody). A poor responder can be treated with a combination of a CD39-neutralizing agent and the agent capable of inducing the extracellular release of ATP from tumor cells.

In certain optional aspects, patients can be identified for treatment with a CD39-neutralizing agent by assessing the presence of extracellular ATP in a tumor sample (e.g., tumor tissue and/or tumor adjacent tissue), optionally wherein a pre-determined concentration of ATP indicates an individual is suitable for treatment with the anti-CD39 agent, optionally wherein the concentration of extracellular ATP is at least 0.01 mM, 0.02 mM, 0.05 mM, 0.125 mM, 0.25 mM or 0.5 mM.

In other embodiments, the treatment methods described herein can be used in combination with any other suitable treatments. In one embodiment, the treatment methods described herein further comprise administering to the individual an agent, optionally an antibody, that neutralizes the inhibitory activity of human PD-1. In one embodiment, the treatment methods described herein further comprise administering to the individual an agent, optionally an antibody, that neutralizes the 5′-ectonucleotidase activity of human CD73 protein.

In other embodiments, pharmaceutical compositions and kits are provided, as well as methods for using them. In one embodiment, provided is a pharmaceutical composition comprising a compound that neutralizes the ATPase activity of a human CD39 polypeptide and an agent capable of inducing the extracellular release of ATP from tumor cells. In one embodiment, provided is a kit comprising a compound that neutralizes the inhibitory activity of a human CD39 polypeptide and an agent capable of inducing the extracellular release of ATP from tumor cells.

In other embodiments, provided are methods for predicting or assessing the efficacy or suitability of an anti-cancer agent for combined use with an antibody that is capable of binding and inhibiting the ATPase activity of a soluble extracellular domain human CD39 protein, the method comprising determining or assessing (e.g., in vitro) whether the anti-cancer agent induces the extracellular release of ATP from cells (e.g. tumor cells), wherein a determination that the anti-cancer agent induces the extracellular release of ATP from cells (e.g. tumor cells) indicates that the agent can be used for treatment of cancer in combination with said antibody that is capable of binding and inhibiting the ATPase activity of a soluble extracellular domain human CD39 protein. Determining or assessing whether the anti-cancer agent induces the extracellular release of ATP from tumor cells can comprise, for example, bringing cells (e.g. tumor cells) into contact with the agent in vitro and assessing extracellular release of ATP.

These aspects are more fully described in, and additional aspects, features, and advantages will be apparent from, the description of the invention provided herein.

### BRIEF DESCRIPTION OF THE DRAWINGS

FIG. 1 shows a representative screening result, showing antibodies I-397, I-398 and I-399 compared to positive control I-394 antibody.

FIG. 2A shows that antibodies BY40, I-394, I-395 and I-396 inhibit cell-membrane bound CD39, with both I-394 and I-395 showing greater potency at all concentrations as well as greater maximal inhibition of cellular CD39 compared to BY40. FIG. 2B shows that antibodies I-395 and I-396 both inhibit soluble CD39 in comparison to negative control (BY40) and positive control (I-394) antibodies.

FIG. 3A shows the position of residues mutated in mutants 5 (M5), 15 (M15) and 19 (M19) on the surface of the CD39 protein. FIG. 3B shows results of binding to mutants 5, 15 and 19 for different antibodies.

FIG. 4 shows binding of antibody I-394 to cells expressing human CD39, as assessed by flow cytometry. I-394 binds cells expressing human CD39 (CHO-huCD39), cells expressing cynomolgus CD39 (CHO-cyCD39) and to Ramos lymphoma cells, but not to cells expressing murine CD39 (CHO-moCD39).

FIG. 5 shows antibody I-394 is highly potent at blocking CD39 enzymatic activity in tumor (Ramos) cells, in cells expressing human CD39 (CHO-huCD39), and in cells expressing cynomolgus CD39 (CHO-cyCD39), as assessed by quantifying luminescence units which are proportional to the amount of ATP present.

FIG. 6 shows antibody I-394 is highly potent at blocking the enzymatic activity of soluble recombinant human CD39 protein, as assessed by quantifying luminescence units which are proportional to the amount of ATP present.

FIG. 7 shows antibody I-394 binds to human CD39 but not to any of the human isoforms CD39-L1, -L2, -L3 or -L4, as assessed in an ELISA assay.

FIG. 8 shows the experimental procedure for assessing the effect of ATP-mediated DC activation on CD4 T cells activation, ATP-activated DC were washed and then incubated with allogenic CD4 T cells (ratio 1 MoDC/4 T cells) for a mixed lymphocytes reaction (MLR) during 5 days. T cells activation and proliferation were analyzed through CD25 expression and Cell Trace Violet dilution by flow cytometry.

FIG. 9 shows HLA-DR expression on moDC and FIG. 10 shows CD83 expression on moDC. These figures show that the anti-CD39 blocking antibody I-394 and chemical inhibitors of CD39 lead to moDC activation at each of 0.125 mM, 0.25 mM or 0.5 mM. However, anti-CD39 antibody BY40 or anti-CD73 antibodies were not able to favor ATP-induced activation of dendritic cell (DC), suggesting that antibodies are not able to block enzymatic activity sufficiently to avoid ATP catabolism. The legends, top to bottom, correspond to the bars in the graph, from left to right.

FIG. 11 shows CD25 expression, showing that MoDC activated in presence of ATP were able to induce T cell activation and proliferation in a MLR assay; the enhancement of ATP-mediated MoDC activation by anti-CD39 blocking antibody I-394 resulted in higher T cell proliferation and activation. The legends, top to bottom, correspond to the bars in the graph, from left to right.

FIG. 12 shows tumor growth and survival in mice treated at day 5 post tumor cell engraftment with either control (1 group) PBS, or oxaliplatin chemotherapy (2 groups). In parallel, one group of mice treated with oxaliplatin was injected twice a week with anti-CD39 antibody, with the anti-CD39 antibody treatment starting just one day before oxaliplatin treatment (at day 4). This ensured that oxaliplatin induced ATP release in a tumor environment where CD39 was already and fully inhibited, and thus providing optimal prevention of ATP degradation by intratumoral CD39.

FIG. 13 shows tumor growth and survival in mice treated at day 5 post tumor cell engraftment with either control (1 group) PBS, anti-CD39 antibody, oxaliplatin, or combination of oxaliplatin and anti-CD39 antibody. The oxaliplatin injection was repeated one week after the first oxaliplatin injection, again just one day after the treatment with I-394 antibody, to provide optimal inhibition of ATP degradation.

### DETAILED DESCRIPTION

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

Human CD39, also known as NTPdasel, ENTPD1, ATPDase and vascular ATP diphosphohydrolase, exhibits ATPase activity. CD39 is a membrane bound protein that hydrolyzes extracellular ATP and ADP to AMP, which is further converted to adenosine by another enzyme, 5-prime nucleotidase. The amino acid sequence of the human CD39 mature polypeptide chain is shown in Genbank under accession number P49961, the entire disclosure of which is incorporated herein by reference, and as follows:

(SEQ ID NO: 1)
MEDTKESNVK TFCSKNILAI LGFSSIIAVI ALLAVGLTQN KALPENVKYG IVLDAGSSHT
SLYIYKWPAE KENDTGVVHQ VEECRVKGPG ISKFVQKVNE IGIYLTDCME RAREVIPRSQ
HQETPVYLGA TAGMRLLRME SEELADRVLD VVERSLSNYP FDFQGARIIT GQEEGAYGWI
TINYLLGKFS QKTRWFSIVP YETNNQETFG ALDLGGASTQ VTFVPQNQII ESPDNALQFR
LYGKDYNVYT HSFLCYGKDQ ALWQKLAKDI QVASNEILRD PCFHPGYKKV VNVSDLYKTP
CTKRFEMTLP FQQFEIQGIG NYQQCHQSIL ELFNTSYCPY SQCAFNGIFL PPLQGDFGAF
SAFYFVMKFL NLTSEKVSQE KVTEMMKKFC AQPWEEIKTS YAGVKEKYLS EYCFSGTYIL
SLLLQGYHFT ADSWEHIHFI GKIQGSDAGW TLGYMLNLTN MIPAEQPLST PLSHSTYVFL
MVLFSLVLFT VAIIGLLIFH KPSYFWKDMV.

In the context herein, “inhibit”, “inhibiting”, “neutralize” or “neutralizing” when referring to the CD39 polypeptide (e.g., “neutralize CD39”, “neutralize the activity of CD39” or “neutralize the enzymatic activity of CD39”, etc.), refers to a process in which the ATP hydrolysis (ATPase) activity of CD39 is inhibited. This comprises, notably the inhibition of CD39-mediated generation of AMP and/or ADP, i.e., the inhibition of CD39-mediated catabolism of ATP to AMP and/or ADP. This can be measured for example in a cellular assay that measures the capacity of a test compound to inhibit the conversion of ATP to AMP and/or ADP, either directly or indirectly. For example, disappearance of ATP and/or generation of AMP can be assessed, as described herein. In one embodiment, an antibody preparation causes at least a 60% decrease in the conversion of ATP to AMP, at least a 70% decrease in the conversion of ATP to AMP, or at least an 80% or 90% decrease in the conversion of ATP to AMP, referring, for example, to the assays described herein (e.g., disappearance of ATP and/or generation of AMP).

“EC₅₀” with respect to an agent and a particular activity (e.g., binding to a cell, inhibition of enzymatic activity, activation or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., CD39, as assessed using either recombinant forms of the proteins, epitopes therein, or native proteins present on the surface of isolated target cells. Competitive binding assays and other methods for determining specific binding are well known in the art. For example binding can be detected via radiolabels, physical methods such as mass spectrometry, or direct or indirect fluorescent labels detected using, e.g., cytofluorometric analysis (e.g., FACScan). Binding above the amount seen with a control, non-specific agent indicates that the agent binds to the target.

When an antibody is said to “compete with” a particular monoclonal antibody, it means that the antibody competes with the monoclonal antibody in a binding assay using either recombinant molecules (e.g., CD39) or surface expressed molecules (e.g., CD39). For example, if a test antibody reduces the binding of an antibody having a heavy chain of SEQ ID NO: 3 and a light chain of SEQ ID NO: 4 to a CD39 polypeptide or CD39-expressing cell in a binding assay, the antibody is said to “compete” respectively with such antibody.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term “conformational” is therefore often used interchangeably with “structural”.

The term “deplete” or “depleting”, with respect to tumor cells, means a process, method, or compound that results in killing, elimination, lysis or induction of such killing, elimination or lysis, so as to negatively affect the number of such tumor cells present in a sample or in a subject.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

Agents that Inhibit CD39

The agent that binds and inhibits CD39 for use in accordance herein can be an antigen binding domain or a protein comprising such, optionally an antibody or antibody fragment, that binds to and inhibits or neutralizes the ATPase activity of a CD39 protein, e.g., a soluble CD39 protein (sCD39), a monomeric CD39 protein, a membrane bound CD39 protein (memCD39), e.g., expressed at the surface of a cell.

In one embodiment a sCD39 protein is a CD39 protein lacking the two transmembrane domains (i.e. the transmembrane domains near the N- and C-terminal ends) found in membrane bound CD39. In one embodiment, sCD39 is a non-membrane bound sCD39 protein found in circulation, e.g., in a human individual. In one embodiment, sCD39 comprises or consists of the amino acid sequence of SEQ ID NO: 2 (optionally further comprising a C-terminal tag or another non-CD39-derived amino acid sequence) optionally wherein the amino acid sequence of SEQ ID NO: 2 further comprises at its N-terminal residues 1 to 37 of the sequence of SEQ ID NO: 1 (i.e. having the N- but not the C-terminal transmembrane domains). The sCD39 protein can also be characterized as comprising or consisting of the Thr38-Va1478 fragment of CD39. In one embodiment, the protein, antibody or antibody fragment inhibits the ATPase activity of sCD39 when incubated with sCD39 in solution, e.g., according to the methods disclosed herein. In one embodiment, the protein, antibody or antibody fragment specifically binds the human CD39 protein, both in soluble (extracellular domain protein) and in membrane-bound form.

In one embodiment, the anti-CD39 antibody does not increase or induce intracellular internalization of, or more generally down-modulation of, cell surface-expressed CD39 and/or does not depend thereupon for its CD39 inhibitory activity.

In one aspect, an anti-CD39 antibody is capable of: (a) inhibiting the enzymatic activity of membrane-bound CD39 protein (e.g., comprising an amino acid sequence of SEQ ID NO: 1) expressed at the surface of cells, and (b) inhibiting the enzymatic activity of soluble CD39 protein (e.g., a CD39 protein having an amino acid sequence of SEQ ID NO: 2, a CD39 protein lacking its transmembrane domains). Examples of antibodies are disclosed herein, including antibodies I-394, I-395, I-396, I-399, 31414, 31895, 31873, 31901 or 31905 (see also PCT publication WO2019/027935 for antibodies and further results of antibodies in inhibition of cell and soluble CD39, the disclosure of which is incorporated herein in its entirety by reference). In one aspect, an anti-CD39 antibody is any antibody available or otherwise known at the filing date of the application disclosing the present invention, or an antibody fragment thereof (e.g. a fragment comprising the heavy and light chain CDRs) that retains the ability to bind CD39 and to inhibit the enzymatic activity of soluble CD39 protein.

In one embodiment, an anti-CD39 antibody does not substantially bind (e.g., via its Fc domain) to human Fcγ receptors (e.g., CD16, CD32a, CD32b, CD64) and/or C1q, and/or do not substantially directing ADCC and/or CDC toward a CD39-expressing cell. Optionally, the antibody retains an Fc domain (e.g., of human IgG isotype) and retains binding to human FcRn.

In one embodiment, the CD39 neutralizing antibodies can be characterized by being capable of causing a decrease in the ATPase activity of a sCD39 polypeptide and/or of a monomeric CD39 polypeptide, optionally causing a decrease of AMP generation by a soluble monomeric human CD39 protein, e.g., a CD39 protein consisting of the amino acid sequence of SEQ ID NO: 2, by at least 50%, 60%, 70%, 80% or 90%.

In one embodiment, the CD39 neutralizing antibodies can be characterized by being capable of causing a decrease in cells' ATPase activity of CD39, optionally causing a decrease of AMP generation by a CD39-expressing cell, by at least 50%, 60%, 70%, 80% or 90%. In one embodiment, the CD39-neutralizing antibodies can be characterized by an EC₅₀ for inhibition of ATPase activity (e.g., EC₅₀ for inhibition of AMP generation by a CD39-expressing cell) of CD39 expressed by a cell of no more than 1 μg/ml, optionally no more than 0.5 μg/ml, optionally no more than 0.2 μg/ml.

In one embodiment, the CD39 neutralizing antibodies can be characterized by being capable of causing an increase in expression of a cell surface marker of activation in human monocyte-derived dendritic cells (moDC), when such moDC are incubated in vitro with the antibody and ATP, optionally wherein ATP is exogenously added ATP, optionally further wherein the added ATP is provided at 0.125 mM, 0.25 mM or 0.5 mM.

An antigen-binding compound can be produced as further described herein, and at any desired stage be assessed for its ability to inhibit the enzymatic activity of CD39, notably to block the ATPase activity of sCD39 and to reduce the production of ADP and AMP (and, together with CD73, adenosine) by soluble CD39 protein and optionally further by a CD39-expressing cell, and in turn restore the ATP-mediated activation of dendritic cell activity and/or T cell proliferation.

The inhibitory activity (e.g., immune enhancing potential) of an antibody can also be assessed for example, in an assay to detect the disappearance (hydrolysis) of ATP and/or the generation of AMP.

The ability of an antibody to inhibit soluble recombinant human CD39 protein can be tested by detecting ATP after incubating test antibody with soluble CD39 protein (e.g., the CD39 protein having the amino acid sequence of SEQ ID NO: 2, as produced in Example 1, optionally further comprising a purification tag or other functional or non-functional non-CD39-derived amino acid sequence). See, e.g., the Examples. Briefly, ATP can be quantified using the Cell Titer Glo™ (Promega), in an assay in which dose ranges of test antibody are incubated with soluble recombinant human CD39 protein described in the Examples, for 1 hour at 37° C. 20 μM ATP are added to the plates for 30 additional minutes at 37° C. before addition of CTG reagent. Emitted light is quantified using an Enspire™ luminometer after a short incubation period of 5 min in the dark.

The ability of an antibody to inhibit cells expressing CD39 protein can be tested by detecting ATP after incubating test antibody with cells (e.g., Ramos cells, cells transfected with CD39, etc.). See, e.g., Examples. Cells can be incubated for 1 hour at 37° C. with test antibody. Cells are then incubated with 20 μM ATP for 1 additional hour at 37° C. Plates are centrifuged for 2 min at 400 g and cell supernatant are transferred in a luminescence microplate (white wells). CTG is added to the supernatant and emitted light is quantified after a 5 min incubation in the dark using an Enspire™ luminometer. Anti-CD39 antibody efficacy is determined by comparing emitted light in presence of antibody with ATP alone (maximal light emission) and ATP together with cells (minimal light emission).

A decrease in hydrolysis of ATP into AMP, and/or an increase of ATP and/or a decrease in generation of AMP, in the presence of antibody indicate the antibody inhibits CD39. In one embodiment, an antibody preparation is capable of causing at least a 60% decrease in the enzymatic activity of a CD39 polypeptide expressed by a cell, preferably the antibody causes at least a 70%, 80% or 90% decrease in the enzymatic activity of a CD39 polypeptide in a cell, as assessed by detecting ATP using the Cell Titer Glo™ (Promega) after incubating cells expressing CD39 polypeptide (e.g., Ramos cells) with a test antibody, e.g., as in Example 1.

In one embodiment, an antibody preparation is capable of causing at least a 60% decrease in the enzymatic activity of a soluble recombinant CD39 polypeptide, preferably at least a 70%, 80% or 90% decrease in the enzymatic activity of a soluble recombinant CD39 polypeptide, as assessed by detecting ATP using the Cell Titer Glo™ (Promega) after incubating soluble recombinant CD39 polypeptide with a test antibody, e.g., as in Example 1.

In one example, provided is an in vitro method for producing or identifying an anti-CD39 antibody or antigen binding domain capable of use in the methods of the disclosure (e.g., for use combination with an agent that induces release of ATP from tumor cells), the method comprising the steps of:

(a) providing a plurality of antibodies that bind a human CD39 polypeptide,

(b) bringing each of the antibodies into contact with human monocyte-derived dendritic cells (moDC), in the presence of ATP, optionally wherein ATP is exogenously added ATP, and

(c) selecting an antibody of step (b) that results in an increase in expression of a cell surface marker of activation in the moDC.

Optionally, in any embodiment herein, a neutralizing anti-CD39 antibody binds an antigenic determinant present on both sCD39 and CD39 expressed at the cell surface (memCD39).

Optionally, in any embodiment herein, a neutralizing anti-CD39 antibody competes for binding to an epitope on CD39 bound by antibody I-394, (e.g., that competes for binding to an epitope on a CD39 polypeptide with an antibody having the heavy and light chain CDRs or variable regions of any of I-394). Optionally, in any embodiment herein, a neutralizing anti-CD39 antibody competes for binding to an epitope on CD39 bound by antibody 31414, 31895, 31873, 31901 or 31905, (e.g., that competes for binding to an epitope on a CD39 polypeptide with an antibody having the heavy and light chain CDRs or variable regions of any of 31414, 31895, 31873, 31901 or 31905).

Optionally, in any embodiment herein, a neutralizing anti-CD39 antibody binds the same epitope and/or competes for binding to a CD39 polypeptide with monoclonal antibody I-394 (e.g., that competes for binding to a CD39 polypeptide with an antibody having the heavy and light chain CDRs or variable regions of I-394. In one embodiment, a neutralizing anti-CD39 antibody binds the same epitope and/or competes for binding to a CD39 polypeptide with an antibody having respectively a VH and VL region of SEQ ID NOS: 3 and 4. Optionally, in any embodiment herein, a neutralizing anti-CD39 antibody binds the same epitope and/or competes for binding to a CD39 polypeptide with monoclonal antibody 31414, 31895, 31873, 31901 or 31905, e.g., that competes for binding to a CD39 polypeptide with an antibody having the heavy and light chain CDRs or variable regions of 31414, 31895, 31873, 31901 or 31905. In one embodiment, a neutralizing anti-CD39 antibody binds the same epitope and/or competes for binding to a CD39 polypeptide with an antibody having respectively a VH and VL region of SEQ ID NOS: 81 and 82.

Optionally, in any embodiment herein, an anti-CD39 antibody binds an epitope comprising one, two or three amino acid residues selected from the group consisting of the amino acid residues on CD39 bound by I-394. Optionally, in any embodiment herein, an anti-CD39 antibody binds an epitope comprising one, two or three amino acid residues selected from the group consisting of the amino acid residues on CD39 bound by 31414, 31895, 31873, 31901 or 31905.

Optionally, in any embodiment herein, the binding molecule (e.g., anti-CD39 antibody) comprises the variable heavy chain domain (V_{H}) comprising a light chain CDR1, 2 and 3 as described herein, and a variable light chain domain (V_{L}) comprising a heavy chain CDR1, 2 and 3 as described herein, or an amino acid sequence in which the CDR (or set of heavy and/or light chain CDRs) has at least 60%, 70%, 80%, 90% or 95% amino acid identity to said CDR (or said set of heavy and/or light chain CDRs). In one aspect of any of the embodiments herein, the antibody may comprise a heavy chain comprising the three CDRs of the heavy chain variable region (VH) of antibody I-394 and a light chain comprising the three CDRs of the light chain variable region (VL) of antibody I-394.

Optionally, in any embodiment herein, anti-CD39 antibody comprises an Fc domain of human IgG4 isotype (optionally modified, e.g. comprising a S228P substitution) or an Fc domain of any human gamma isotype (e.g, IgG1, IgG2, IgG3, IgG4) that is modified (compared to a wild-type Fc domain of the same isotype) to reduce binding between the Fc domain and human CD16A, CD16B, CD32A, CD32B and/or CD64 polypeptides, optionally wherein the antibody comprises: (i) a heavy chain comprising CDR 1, 2 and 3 of the heavy chain variable region of SEQ ID NO: 3 and (ii) a light chain comprising CDR 1, 2 and 3 of the light chain variable region of SEQ ID NO: 4. In one aspect, the Fc domain is modified (compared to a wild-type Fc domain of the same isotype) to reduce binding between the Fc domain and human C1q polypeptide. In one embodiment, the antibody comprises an amino acid substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330 and 331 (Kabat EU numbering). In one embodiment, the antibody has an amino acid substitution in a heavy chain constant region at any three, four, five or more of residues selected from the group consisting of: 234, 235, 237, 322, 330 and 331. In one embodiment, the antibody comprises an Fc domain comprising an amino acid sequence shown below.

In one embodiment, an antibody comprises a heavy chain constant region or Fc domain comprising the amino acid sequence below, or an amino acid sequence at least 90%, 95% or 99% identical thereto but retaining the amino acid residues at Kabat positions 234, 235 and 331 (underlined):

(SEQ ID NO: 44)
A S T K G P S V F P L A P S S K S T S G G T A A L G
C L V K D Y F P E P V T V S W N S G A L T S G V H T
F P A V L Q S S G L Y S L S S V V T V P S S S L G T
Q T Y I C N V N H K P S N T K V D K R V E P K S C D
K T H T C P P C P A P E A E G G P S V F L F P P K P
K D T L M I S R T P E V T C V V V D V S H E D P E V
K F N W Y V D G V E V H N A K T K P R E E Q Y N S T
Y R V V S V L T V L H Q D W L N G K E Y K C K V S N
K A L P A S I E K T I S K A K G Q P R E P Q V Y T L
P P S R E E M T K N Q V S L T C L V K G F Y P S D I
A V E W E S N G Q P E N N Y K T T P P V L D S D G S
F F L Y S K L T V D K S R W Q Q G N V F S C S V M H
E A L H N H Y T Q K S L S L S P G K

In one embodiment, an antibody comprises a heavy chain constant region or Fc domain comprising the amino acid sequence below, or an amino acid sequence at least 90%, 95% or 99% identical thereto but retaining the amino acid residues at Kabat positions 234, 235 and 331 (underlined):

(SEQ ID NO: 45)
A S T K G P S V F P L A P S S K S T S G G T A A L G
C L V K D Y F P E P V T V S W N S G A L T S G V H T
F P A V L Q S S G L Y S L S S V V T V P S S S L G T
Q T Y I C N V N H K P S N T K V D K R V E P K S C D
K T H T C P P C P A P E F E G G P S V F L F P P K P
K D T L M I S R T P E V T C V V V D V S H E D P E V
K F N W Y V D G V E V H N A K T K P R E E Q Y N S T
Y R V V S V L T V L H Q D W L N G K E Y K C K V S N
K A L P A S I E K T I S K A K G Q P R E P Q V Y T L
P P S R E E M T K N Q V S L T C L V K G F Y P S D I
A V E W E S N G Q P E N N Y K T T P P V L D S D G S
F F L Y S K L T V D K S R W Q Q G N V F S C S V M H
E A L H N H Y T Q K S L S L S P G K

In one embodiment, an antibody comprises a heavy chain constant region or Fc domain comprising the amino acid sequence below, or an amino acid sequence at least 90%, 95% or 99% identical thereto but retaining the amino acid residues at Kabat positions 234, 235, 237, 330 and 331 (underlined):

(SEQ ID NO: 46)
A S T K G P S V F P L A P S S K S T S G G T A A L G
C L V K D Y F P E P V T V S W N S G A L T S G V H T
F P A V L Q S S G L Y S L S S V V T V P S S S L G T
Q T Y I C N V N H K P S N T K V D K R V E P K S C D
K T H T C P P C P A P E A E G A P S V F L F P P K P
K D T L M I S R T P E V T C V V V D V S H E D P E V
K F N W Y V D G V E V H N A K T K P R E E Q Y N S T
Y R V V S V L T V L H Q D W L N G K E Y K C K V S N
K A L P S S I E K T I S K A K G Q P R E P Q V Y T L
P P S R E E M T K N Q V S L T C L V K G F Y P S D I
A V E W E S N G Q P E N N Y K T T P P V L D S D G S
F F L Y S K L T V D K S R W Q Q G N V F S C S V M H
E A L H N H Y T Q K S L S L S P G K

In one embodiment, an antibody comprises a heavy chain constant region or Fc domain comprising the amino acid sequence below, or a sequence at least 90%, 95% or 99% identical thereto but retaining the amino acid residues at Kabat positions 234, 235, 237 and 331 (underlined):

(SEQ ID NO: 47)
A S T K G P S V F P L A P S S K S T S G G T A A L G
C L V K D Y F P E P V T V S W N S G A L T S G V H T
F P A V L Q S S G L Y S L S S V V T V P S S S L G T
Q T Y I C N V N H K P S N T K V D K R V E P K S C D
K T H T C P P C P A P E A E G A P S V F L F P P K P
K D T L M I S R T P E V T C V V V D V S H E D P E V
K F N W Y V D G V E V H N A K T K P R E E Q Y N S T
Y R V V S V L T V L H Q D W L N G K E Y K C K V S N
K A L P A S I E K T I S K A K G Q P R E P Q V Y T L
P P S R E E M T K N Q V S L T C L V K G F Y P S D I
A V E W E S N G Q P E N N Y K T T P P V L D S D G S
F F L Y S K L T V D K S R W Q Q G N V F S C S V M H
E A L H N H Y T Q K S L S L S P G K

In one aspect, the anti-CD39 antibody binds the same epitope as antibody I-394, I-395, I-396, I-397, I-398 or I-399. In one aspect, the anti-CD39 antibody binds the same epitope as antibody 31414, 31895, 31873, 31901 or 31905. In one embodiment, the antibodies bind to an epitope of CD39 that at least partially overlaps with, or includes at least one residue in, the epitope bound by antibody I-394, I-395, I-396, I-397, I-398 or I-399. The residues bound by the antibody can be specified as being present on the surface of the CD39 polypeptide, e.g., in a CD39 polypeptide expressed on the surface of a cell.

Binding of anti-CD39 antibody to cells transfected with CD39 mutants can be measured and compared to the ability of anti-CD39 antibody to bind wild-type CD39 polypeptide (e.g., SEQ ID NO: 1). A reduction in binding between an anti-CD39 antibody and a mutant CD39 polypeptide (e.g., a mutant of Table 1) means that there is a reduction in binding affinity (e.g., as measured by known methods such FACS testing of cells expressing a particular mutant, or by Biacore testing of binding to mutant polypeptides) and/or a reduction in the total binding capacity of the anti-CD39 antibody (e.g., as evidenced by a decrease in Bmax in a plot of anti-CD39 antibody concentration versus polypeptide concentration). A significant reduction in binding indicates that the mutated residue is directly involved in binding to the anti-CD39 antibody or is in close proximity to the binding protein when the anti-CD39 antibody is bound to CD39.

In some embodiments, a significant reduction in binding means that the binding affinity and/or capacity between an anti-CD39 antibody and a mutant CD39 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type CD39 polypeptide. In certain embodiments, binding is reduced below detectable limits. In some embodiments, a significant reduction in binding is evidenced when binding of an anti-CD39 antibody to a mutant CD39 polypeptide is less than 50% (e.g., less than 45%, 40%, 35%, 30%, 25%, 20%, 15% or 10%) of the binding observed between the anti-CD39 antibody and a wild-type CD39 polypeptide.

In some embodiments, anti-CD39 antibodies exhibit significantly lower binding for a mutant CD39 polypeptide in which a residue in a segment comprising an amino acid residue bound by antibody I-394, I-395, I-396, I-397, I-398 or I-399 is substituted with a different amino acid. In some embodiments, anti-CD39 antibodies exhibit significantly lower binding for a mutant CD39 polypeptide in which a residue in a segment comprising an amino acid residue bound by antibody 31414, 31895, 31873, 31901 or 31905 is substituted with a different amino acid.

In some embodiments, anti-CD39 antibodies (e.g., other than I-394) are provided that bind the epitope on CD39 bound by antibody I-394, I-395, I-396, I-397, I-398 or I-399. In some embodiments, anti-CD39 antibodies (e.g., other than I-394) are provided that bind the epitope on CD39 bound by antibody 31414, 31895, 31873, 31901 or 31905.

In one aspect, the antibody binds an epitope on CD39 comprising an amino acid residue (e.g., one, two or three of the residues) selected from the group consisting of R138, M139 and E142 (with reference to SEQ ID NO: 1).

In one aspect, an anti-CD39 antibody exhibits reduced binding (e.g. substantially complete loss of binding) to a CD39 polypeptide having a mutation at one, two or three of the residues selected from the group consisting of: R138, M139 and E142 (with reference to SEQ ID NO: 1), compared to a wild-type CD39 polypeptide (a CD39 polypeptide of SEQ ID NO: 1); optionally, the mutant CD39 polypeptide has the mutations: R138A, M139A and E142K. In one optional aspect, the antibody does not have a loss of binding to any of the mutant CD39 polypeptide of Table 1 other than mutant 19. In another optional aspect, the anti-CD39 antibody exhibits reduced binding (optionally reduced but not a substantially complete loss of binding; or optionally a substantially complete loss of binding) to a CD39 polypeptide having a mutation at one, two, three or four of the residues selected from the group consisting of: Q96, N99, E143 and R147 (with reference to SEQ ID NO: 1), compared to a wild-type CD39 polypeptide (a CD39 polypeptide of SEQ ID NO: 1); optionally, the mutant CD39 polypeptide has the mutations: Q96A, N99A, E143A and R147E.

In one aspect, the antibody binds an epitope on CD39 comprising an amino acid residue (e.g., one, two, three or four of the residues) selected from the group consisting of Q96, N99, E143 and R147 (with reference to SEQ ID NO: 1). In one aspect, the antibody has reduced binding (e.g. substantially complete loss of binding) to a mutant CD39 polypeptide comprising a mutation at 1, 2, 3 or 4 residues selected from the group consisting of Q96, N99, E143 and R147 (with reference to SEQ ID NO: 1), in each case relative to binding between the antibody and a wild-type CD39 polypeptide comprising the amino acid sequence of SEQ ID NO: 1.

In one aspect, the antibody binds an epitope on CD39 comprising (a) an amino acid residue (e.g., one, two or three of the residues) selected from the group consisting of R138, M139 and E142 (with reference to SEQ ID NO: 1), and (b) an amino acid residue (e.g., one, two, three or four of the residues) selected from the group consisting of Q96, N99, E143 and R147.

In one aspect, an anti-CD39 antibody exhibits reduced (e.g. substantially complete loss of) binding to both (a) a CD39 polypeptide having a mutation at one, two, three or four of the residues selected from the group consisting of: Q96, N99, E143 and R147 (with reference to SEQ ID NO: 1), and (b) a CD39 polypeptide having a mutation at one, two, or three of the residues selected from the group consisting of: R138, M139 and E142 (with reference to SEQ ID NO: 1), in each case compared to a wild-type CD39 polypeptide (a CD39 polypeptide of SEQ ID NO: 1). Optionally, the mutant CD39 polypeptide of (a) has the mutations: Q96A, N99A, E143A and R147E. Optionally, the mutant CD39 polypeptide of (b) has the mutations: R138A, M139A and E142K. Optionally the antibody does not have a loss of binding to any of the mutant CD39 polypeptide of Table 1 other than mutants 5 and 19.

In one aspect, the antibody binds an epitope on CD39 comprising an amino acid residue (e.g., one, two, three or four of the residues) selected from the group consisting of K87, E100 and D107 (with reference to SEQ ID NO: 1).

In one aspect, an anti-CD39 antibody exhibits reduced binding (e.g. substantially complete loss of binding) to a CD39 polypeptide having a mutation at one, two, three or four of the residues selected from the group consisting of: K87, E100 and D107 (with reference to SEQ ID NO: 1), compared to a wild-type CD39 polypeptide (a CD39 polypeptide of SEQ ID NO: 1); optionally, the mutant CD39 polypeptide has the mutations: K87A, E100A and D107A. Optionally the antibody does not have a loss of binding to any of the mutant CD39 polypeptide of Table 1 other than mutant 15.

In one aspect, the antibody binds an epitope on CD39 comprising an amino acid residue (e.g., one, two, three or four of the residues) selected from the group consisting of N371, L372, E375, K376 and V377 (with reference to SEQ ID NO: 1).

In one aspect, an anti-CD39 antibody exhibits reduced (e.g. substantially complete loss of) binding to a CD39 polypeptide having a mutation at one, two, three, four or five of the residues selected from the group consisting of: N371, L372, E375, K376 and V377 (with reference to SEQ ID NO: 1), compared to a wild-type CD39 polypeptide (a CD39 polypeptide of SEQ ID NO: 1); optionally, the mutant CD39 polypeptide has the mutations: N371K, L372K, E375A, K376G and V377S, and an insertion of a valine between residues 376 and 377. Optionally the antibody does not have a loss of binding to any of the mutant CD39 polypeptide of Table 1 other than mutant 11.

In one aspect, the antibody binds to residues bound by 31414, 31895, 31873, 31901 and/or 31905 antibodies. In one aspect, the antibody binds to residues in a segment of CD39 comprising residues 143-158 (with reference to SEQ ID NO: 1). In one aspect, the antibody binds an epitope on CD39 comprising an amino acid residue (e.g., one, two, three, four, five or more of the residues) selected from the group consisting of residues 143-158 (with reference to SEQ ID NO: 1). In one aspect, an anti-CD39 antibody exhibits reduced binding (e.g. substantially complete loss of binding) to a CD39 polypeptide having a mutation at one, two or three of the residues selected from the group consisting of: D150, E153 and R154 (with reference to SEQ ID NO: 1), compared to a wild-type CD39 polypeptide (a CD39 polypeptide of SEQ ID NO: 1). In one aspect, an anti-CD39 antibody exhibits reduced binding (e.g. substantially complete loss of binding) to a CD39 polypeptide having a mutation at one, two or three of the residues selected from the group consisting of: N99, E153 and R154 (with reference to SEQ ID NO: 1), compared to a wild-type CD39 polypeptide (a CD39 polypeptide of SEQ ID NO: 1). In one aspect, an anti-CD39 antibody exhibits reduced binding (e.g. substantially complete loss of binding) to a CD39 polypeptide having a mutation at one, two, three or four of the residues selected from the group consisting of: N99, D150, E153 and R154 (with reference to SEQ ID NO: 1), compared to a wild-type CD39 polypeptide (a CD39 polypeptide of SEQ ID NO: 1).

An anti-CD39 antibody may for example comprise: a HCDR1 comprising an amino acid sequence: DYNMH (SEQ ID NO: 5), or a sequence of at least 4 contiguous amino acids thereof, optionally wherein one or more of these amino acids may be substituted by a different amino acid; a HCDR2 comprising an amino acid sequence: YIVPLNGGSTFNQKFKG (SEQ ID NO: 6), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GGTRFAY (SEQ ID NO: 7), or a sequence of at least 4, 5 or 6 contiguous amino acids thereof, optionally wherein one or more of these amino acids may be substituted by a different amino acid; a LCDR1 comprising an amino acid sequence: RASESVDNFGVSFMY (SEQ ID NO: 8), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: GASNQGS (SEQ ID NO: 9) or a sequence of at least 4, 5 or 6 contiguous amino acids thereof, optionally wherein one or more of these amino acids may be substituted by a different amino acid; and/or a LCDR3 region of I-394 comprising an amino acid sequence: QQTKEVPYT (SEQ ID NO: 10), or a sequence of at least 4, 5, 6, 7 or 8 contiguous amino acids thereof, optionally wherein one or more of these amino acids may be deleted or substituted by a different amino acid. CDR positions may be according to Kabat numbering.

An exemplary anti-CD39 VH and VL pair of an antibody that inhibits the enzymatic activity of human sCD39 protein is that of antibody I-394, the amino acid sequence of the heavy chain variable region of which is listed below (SEQ ID NO: 3), and the amino acid sequence of the light chain variable region of which is listed below (SEQ ID NO: 4). The CDRs according to Kabat numbering are underlined in SEQ ID NOS: 3 and 4. Optionally, the VH and VL comprise (e.g., are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 3. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 4.

I-394 VH:
(SEQ ID NO: 3)
EVQLQQSGPELVKPGASVKMSCKASGYTFTDYNMHWVKQSHGRTLEWIGYI
VPLNGGSTFNQKFKGRATLTVNTSSRTAYMELRSLTSEDSAAYYCARGGTR
FAYWGQGTLVTVSA.
I-394 VL:
(SEQ ID NO: 4)
DIVLTQSPASLAVSLGQRATISCRASESVDNFGVSFMYWFQQKPGQPPNLL
IYGASNQGSGVPARFRGSGSGTDFSLNIHPMEADDTAMYFCQQTKEVPYTF
GGGTKLEIK.

Another exemplary anti-CD39 VH and VL pair according to the disclosure is that of antibody I-395, the amino acid sequence of the heavy chain variable region of which is listed below (SEQ ID NO: 11), and the amino acid sequence of the light chain variable region of which is listed below (SEQ ID NO: 12). The CDRs according to Kabat numbering are underlined in SEQ ID NOS: 11 and 12. Optionally, the VH and VL comprise (e.g., are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 11. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 12.

I-395 VH:
(SEQ ID NO: 11)
EVQLQQSGPELVKPGASVRMSCKASGYTFTDYNMHWVKKNHGKGLEWIGY
INPNNGGTTYNQKFKGKATLTVNTSSKTAYMELRSLTSEDSAVYYCTRGG
TRFASWGQGTLVTVSA.
I-395 VL:
(SEQ ID NO: 12)
NIVLTQSPASLAVSLGQRATISCRASESVDNYGISFMYWFQQKPGQPPKL
LIYAASTQGSGVPARFSGSGSGTDFSLNIHPMEEDDTAMYFCQQSKEVPF
TFGSGTKLEIK.

An anti-CD39 antibody may for example comprise: a HCDR1 of I-395 comprising an amino acid sequence: DYNMH (SEQ ID NO: 13), or a sequence of at least 4 contiguous amino acids thereof, optionally wherein one or more of these amino acids may be substituted by a different amino acid; a HCDR2 of I-395 comprising an amino acid sequence: YINPNNGGTTYNQKFKG (SEQ ID NO: 14), or a sequence of at least 4, 5, 6, 7, 8, 9 or 10 contiguous amino acids thereof, optionally wherein one or more of these amino acids may be substituted by a different amino acid; a HCDR3 of I-395 comprising an amino acid sequence: GGTRFAS (SEQ ID NO: 15), or a sequence of at least 4, 5, 6 contiguous amino acids thereof, optionally wherein one or more of these amino acids may be substituted by a different amino acid; a LCDR1 of I-395 comprising an amino acid sequence: RASESVDNYGISFMY (SEQ ID NO: 16), or a sequence of at least 4, 5, 6, 7, 8, 9 or 10 contiguous amino acids thereof, optionally wherein one or more of these amino acids may be substituted by a different amino acid; a LCDR2 region of I-395 comprising an amino acid sequence: AASTQGS (SEQ ID NO: 17) or a sequence of at least 4, 5 or 6 contiguous amino acids thereof, optionally wherein one or more of these amino acids may be substituted by a different amino acid; and/or a LCDR3 region of I-395 comprising an amino acid sequence: QQSKEVPFT (SEQ ID NO: 18), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody I-396, the amino acid sequence of the heavy chain variable region of which is listed below (SEQ ID NO: 19), and the amino acid sequence of the light chain variable region of which is listed below (SEQ ID NO: 20). The CDRs according to Kabat numbering are underlined in SEQ ID NOS: 19 and 20. Optionally, the VH and VL comprise (e.g., are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 19. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 20.

I-396 VH:
(SEQ ID NO: 19)
EVQLQQSGAELVKPGASVKLSCIVSGFNIKDTYINWVKQRPEQGLEWIGR
IDPANGNTKYDPKFQGKATMTSDTSSNTAYLHLSSLTSDDSAVYYCARWG
YDDEEADYFDSWGQGTTLTVSS.
I-396 VL:
(SEQ ID NO: 20)
DIVLTQSPASLAVSLGQRATISCRASESVDNYGISFMNWFQQKPGQPPKL
LIYAASNQGSGVPARFSGSGSGTDFSLNILPMEEVDAAMYFCHQSKEVPW
TFGGGTKLEIK.

An anti-CD39 antibody may for example comprise: a HCDR1 comprising an amino acid sequence: DTYIN (SEQ ID NO: 21), or a sequence of at least 4 contiguous amino acids thereof, optionally wherein one or more of these amino acids may be substituted by a different amino acid; a HCDR2 comprising an amino acid sequence: RIDPANGNTKYDPKFQG (SEQ ID NO: 22), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: WGYDDEEADYFDS (SEQ ID NO: 23), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: RASESVDNYGISFMN (SEQ ID NO: 24), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: AASNQGS (SEQ ID NO: 25) or a sequence of at least 4, 5 or 6 contiguous amino acids thereof, optionally wherein one or more of these amino acids may be substituted by a different amino acid; and/or a LCDR3 region of I-396 comprising an amino acid sequence: HQSKEVPWT (SEQ ID NO: 26), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody I-399, the amino acid sequence of the heavy chain variable region of which is listed below (SEQ ID NO: 27), and the amino acid sequence of the light chain variable region of which is listed below (SEQ ID NO: 28). The CDRs according to Kabat numbering are underlined in SEQ ID NOS: 27 and 28. Optionally, the VH and VL comprise (e.g., are modified to incorporate) human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 27. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 28.

I-399 VH:
(SEQ ID NO: 27)
PVQLQQPGAEVVMPGASVKLSCKASGYTFTSFWMNWMRQRPGQGLEWIGE
IDPSDFYTNSNQRFKGKATLTVDKSSSTAYMQLSSLTSEDSAVYFCARGD
FGWYFDVWGTGTSVTVSS.
I-399 VL:
(SEQ ID NO: 28)
EIVLTQSPTTMTSSPGEKITFTCSASSSINSNYLHWYQQKPGFSPKLLIY
RTSNLASGVPTRFSGSGSGTSYSLTIGTMEAEDVATYYCQQGSSLPRTFG
GGTKLEIK.

An anti-CD39 antibody may for example comprise: a HCDR1 comprising an amino acid sequence: SFWMN (SEQ ID NO: 29), or a sequence of at least 4 contiguous amino acids thereof, optionally wherein one or more of these amino acids may be substituted by a different amino acid; a HCDR2 comprising an amino acid sequence: EIDPSDFYTNSNQRFKG (SEQ ID NO: 30), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GDFGWYFDV (SEQ ID NO: 31), or a sequence of at least 4, 5 or 6 contiguous amino acids thereof, optionally wherein one or more of these amino acids may be substituted by a different amino acid; a LCDR1 comprising an amino acid sequence: SASSSINSNYLH (SEQ ID NO: 32), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: RTSNLAS (SEQ ID NO: 33) or a sequence of at least 4, 5 or 6 contiguous amino acids thereof, optionally wherein one or more of these amino acids may be substituted by a different amino acid; and/or a LCDR3 region of I-399 comprising an amino acid sequence: QQGSSLPRT (SEQ ID NO: 34), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody 31905, the amino acid sequence of the heavy chain variable region of which is listed below (SEQ ID NO: 49), and the amino acid sequence of the light chain variable region of which is listed below (SEQ ID NO: 50). The CDRs according to Kabat numbering are underlined in SEQ ID NOS: 49 and 50. The VH and VL can be specified as comprising human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 49. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 50.

31905 VH:
(SEQ ID NO: 49)
QVQLVQSGAEVKKPGSSVKVSCKASGGTFPSNAISWVRQAPGQGLEWMGG
IGFGTANYAQKFQGRVTITADESTSTAYMELSSLRSEDTAVYYCARGGAK
YARTYGMDVWGQGTTVTVSS.
31905 VL:
(SEQ ID NO: 50)
DIVMTQSPDSLAVSLGERATINCKSSKSVLYSNNNKNYLAWYQQKPGQPP
KLLIYWASTRQSGVPDRFSGSGSGTDFTLTISSLQAEDVAVYYCQQYLLY
PLTFGGGTKVEIK.

An anti-CD39 antibody may for example comprise: a HCDR1 comprising an amino acid sequence: SNAIS (SEQ ID NO: 51), or a sequence of at least 4 contiguous amino acids thereof, optionally wherein one or more of these amino acids may be substituted by a different amino acid; a HCDR2 comprising an amino acid sequence: GIGFGTANYAQKFQG (SEQ ID NO: 52), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GGAKYARTYGMDV (SEQ ID NO: 53), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: KSSKSVLYSNNNKNYLA (SEQ ID NO: 54), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: WASTRQS (SEQ ID NO: 55) or a sequence of at least 4, 5 or 6 contiguous amino acids thereof, optionally wherein one or more of these amino acids may be substituted by a different amino acid; and/or a LCDR3 region comprising an amino acid sequence: QQYLLYPLT (SEQ ID NO: 56), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody 31901, the amino acid sequence of the heavy chain variable region of which is listed below (SEQ ID NO: 57), and the amino acid sequence of the light chain variable region of which is listed below (SEQ ID NO: 58). The CDRs according to Kabat numbering are underlined in SEQ ID NOS: 57 and 58. The VH and VL can be specified as comprising human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 57. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 58.

31901 VH:
(SEQ ID NO: 57)
QVQLVQSGAEVKKPGSSVKVSCKASGGTFSSLPISWVRQAPGQGLEWMGG
IGFGTANYAQKFQGRVTITADESTSTAYMELSSLRSEDTAVYYCARGGAK
YAGRYGMDVWGQGTTVTVSS.
31901 VL:
(SEQ ID NO: 58)
GIVMTQSPDSLAVSLGERATINCKSSQSVLFSSNNKNYLAWYQQKPGQPP
KLLIYWASTRASGVPDRFSGSGSGTDFTLTISSLQAEDVAVYYCQQYYLY
PLTFGGGTKVEIK.

An anti-CD39 antibody may for example comprise: a HCDR1 comprising an amino acid sequence: SLPIS (SEQ ID NO: 59), or a sequence of at least 4 contiguous amino acids thereof, optionally wherein one or more of these amino acids may be substituted by a different amino acid; a HCDR2 comprising an amino acid sequence: GIGFGTANYAQKFQ (SEQ ID NO: 60), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GGAKYAGRYGMDV (SEQ ID NO: 61), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: KSSQSVLFSSNNKNYLA (SEQ ID NO: 62), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: WASTRAS (SEQ ID NO: 63) or a sequence of at least 4, 5 or 6 contiguous amino acids thereof, optionally wherein one or more of these amino acids may be substituted by a different amino acid; and/or a LCDR3 region comprising an amino acid sequence: QQYYLYPLT (SEQ ID NO: 64), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody 31873, the amino acid sequence of the heavy chain variable region of which is listed below (SEQ ID NO: 65), and the amino acid sequence of the light chain variable region of which is listed below (SEQ ID NO: 66). The CDRs according to Kabat numbering are underlined in SEQ ID NOS: 65 and 66. The VH and VL can be specified as comprising human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 65. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 66.

31873 VH:
(SEQ ID NO: 65)
QVQLVQSGAEVKKPGSSVKVSCKASGGTFSKYGISWVRQAPGQGLEWMGS
IIPEFGIANYAQKFQGRVTITADESTSTAYMELSSLRSEDTAVYYCARES
GGYRDHRLGVWGQGTMVTVSS.
31873 VL:
(SEQ ID NO: 66)
EIVMTQSPATLSVSPGERATLSCRASQSVGSNLAWYQQKPGQAPRLLIYG
ASTRASGIPARFSGSGSGTEFTLTISSLQSEDFAVYYCQQYLLWPLTFGG
GTKVEIK.

An anti-CD39 antibody may for example comprise: a HCDR1 comprising an amino acid sequence: KYGIS (SEQ ID NO: 67), or a sequence of at least 4 contiguous amino acids thereof, optionally wherein one or more of these amino acids may be substituted by a different amino acid; a HCDR2 comprising an amino acid sequence: SIIPEFGIANYAQKFQG (SEQ ID NO: 68), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: ESGGYRDHRLGV (SEQ ID NO: 69), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: RASQSVGSNLA (SEQ ID NO: 70), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: GASTRAS (SEQ ID NO: 71) or a sequence of at least 4, 5 or 6 contiguous amino acids thereof, optionally wherein one or more of these amino acids may be substituted by a different amino acid; and/or a LCDR3 region comprising an amino acid sequence: QQYLLWPLT (SEQ ID NO: 72), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody 31414, the amino acid sequence of the heavy chain variable region of which is listed below (SEQ ID NO: 73), and the amino acid sequence of the light chain variable region of which is listed below (SEQ ID NO: 74). The CDRs according to Kabat numbering are underlined in SEQ ID NOS: 73 and 74. The VH and VL can be specified as comprising human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 73. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 74.

31414 VH:
(SEQ ID NO: 73)
QVQLVQSGAEVKKPGASVKVSCKASGYTFKSYEMHWVRQAPGQGLEWMGR
INPSVGSTWYAQKFQGRVTMTRDTSTSTVYMELSSLRSEDTAVYYCARGK
REGGTEYLRNWGQGTLVTVSS.
31414 VL:
(SEQ ID NO: 74)
EIVLTQSPGTLSLSPGERATLSCRASQSVSSSYLAWYQQKPGQAPRLLIY
GASSRATGIPDRFSGSGSGTDFTLTISRLEPEDFAVYYCQQYHSYITFGG
GTKVEIK.

An anti-CD39 antibody may for example comprise: a HCDR1 comprising an amino acid sequence: SYEMH (SEQ ID NO: 75), or a sequence of at least 4 contiguous amino acids thereof, optionally wherein one or more of these amino acids may be substituted by a different amino acid; a HCDR2 comprising an amino acid sequence: RINPSVGSTWYAQKFQG (SEQ ID NO: 76), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GKREGGTEYLRN (SEQ ID NO: 77), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: RASQSVSSSYLA (SEQ ID NO: 78), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: GASSRAT (SEQ ID NO: 79) or a sequence of at least 4, 5 or 6 contiguous amino acids thereof, optionally wherein one or more of these amino acids may be substituted by a different amino acid; and/or a LCDR3 region comprising an amino acid sequence: QQYHSYIT (SEQ ID NO: 80), or a sequence of at least 4, 5, 6, 7 or 8 contiguous amino acids thereof, optionally wherein one or more of these amino acids may be deleted or substituted by a different amino acid. CDR positions may be according to Kabat numbering.

Another exemplary anti-CD39 VH and VL pair according to the disclosure is that of antibody 31895, the amino acid sequence of the heavy chain variable region of which is listed below (SEQ ID NO: 81), and the amino acid sequence of the light chain variable region of which is listed below (SEQ ID NO: 82). The CDRs according to Kabat numbering are underlined in SEQ ID NOS: 81 and 82. The VH and VL can be specified as comprising human acceptor frameworks. In one embodiment, an anti-CD39 antibody of the disclosure comprises the VH CDR1, CDR2 and/or CDR3 (e.g., according to Kabat numbering) of the heavy chain variable region having the amino acid sequence of SEQ ID NO: 81. In one embodiment, an anti-CD39 antibody of the disclosure comprise the VL CDR1, CDR2 and/or CDR3 (e.g., according to Kabat numbering) of the light chain variable region having the amino acid sequence of SEQ ID NO: 82.

31895 VH:
(SEQ ID NO: 81)
QVQLVQSGAEVKKPGASVKVSCKASGYTFKSYEMHWVRQAPGQGLEWMGR
INPSVGSTWYAQKFQGRVTMTRDTSTSTVYMELSSLRSEDTAVYYCARGK
REGGTEYLRKWGQGTLVTVSS.
31895 VL:
(SEQ ID NO: 82)
EIVLTQSPGTLSLSPGERATLSCRASQSVASSYLAWYQQKPGQAPRLLIY
GASNRHTGIPDRFSGSGSGTDFTLTISRLEPEDFAVYYCQQYHNAITFGG
GTKVEIK.

An anti-CD39 antibody may for example comprise: a HCDR1 comprising an amino acid sequence: SYEMH (SEQ ID NO: 83), or a sequence of at least 4 contiguous amino acids thereof, optionally wherein one or more of these amino acids may be substituted by a different amino acid; a HCDR2 comprising an amino acid sequence: RINPSVGSTWYAQKFQG (SEQ ID NO: 84), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GKREGGTEYLRK (SEQ ID NO: 85), or a sequence of at least 4, 5, 6, 7, 8, 9 or 10 contiguous amino acids thereof, optionally wherein one or more of these amino acids may be substituted by a different amino acid; a LCDR1 comprising an amino acid sequence: RASQSVASSYLA (SEQ ID NO: 86), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: GASNRHT (SEQ ID NO: 87) or a sequence of at least 4, 5 or 6 contiguous amino acids thereof, optionally wherein one or more of these amino acids may be substituted by a different amino acid; and/or a LCDR3 region comprising an amino acid sequence: QQYHNAIT (SEQ ID NO: 88), or a sequence of at least 4, 5, 6, 7 or 8 contiguous amino acids thereof, optionally wherein one or more of these amino acids may be deleted or substituted by a different amino acid. CDR positions may be according to Kabat numbering.

An exemplary 31895 antibody may have a heavy chain comprising the amino acid sequence of SEQ ID NO 89, shown below, and a light chain comprising the amino acid sequence of SEQ ID NO: 90, shown below.

31895 heavy chain:
(SEQ ID NO: 89)
QVQLVQSGAEVKKPGASVKVSCKASGYTFKSYEMHWVRQAPGQGLEWMGR
INPSVGSTWYAQKFQGRVIMTRDTSTSTVYMELSSLRSEDTAVYYCARGK
REGGTEYLRKWGQGTLVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
31895 light chain:
(SEQ ID NO: 90)
EIVLTQSPGTLSLSPGERATLSCRASQSVASSYLAWYQQKPGQAPRLLIY
GASNRHTGIPDRFSGSGSGTDFTLTISRLEPEDFAVYYCQQYHNAITFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In any of the I-394, I-395, I-396, I-399, 31414, 31895, 31873, 31901 or 31905 antibodies, the HCDR1, 2, 3 and LCDR1, 2, 3 sequences (each CDR independently, or all CDRs) can be specified as being those of the Kabat numbering system, (as indicated in the VH and VL sequences by underlining), those of the Chothia numbering system, or, those of the IMGT numbering system, or any other suitable numbering system.

In any aspect, the specified variable region, FR and/or CDR sequences may comprise one or more sequence modifications, e.g., a substitution (1, 2, 3, 4, 5, 6, 7, 8 or more sequence modifications). In one embodiment the substitution is a conservative modification.

In another aspect, the anti-CD39 compound comprises a VH domain having at least about 60%, 70% or 80% sequence identity, optionally at least about 85%, 90%, 95%, 97%, 98% or 99% identity, to the VH domain of an antibody disclosed herein. In another aspect, the anti-CD39 antibody comprises a VL domain having at least about 60%, 70% or 80% sequence identity, optionally at least about 85%, 90%, 95%, 97%, 98% or 99% identity, to the VL domain of an antibody disclosed herein.

Optionally, in any embodiment herein, an anti-CD39 antibody can be characterized as being a function-conservative variant of any of the antibodies, heavy and/or light chains, CDRs or variable regions thereof described herein. “Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein (e.g. heavy or light chains, or CDRs or variable regions thereof) to which it is compared. In one embodiment, the antibody comprises a heavy chain variable region that is a function-conservative variant of the heavy chain variable region of antibody I-394, I-395, I-396, I-397, I-398 or I-399, and a light chain variable region that is a function-conservative variant of the light chain variable region of the respective I-394, I-395, I-396, I-397, I-398 or I-399 antibody. In one embodiment, the antibody comprises a heavy chain that is a function-conservative variant of the heavy chain variable region of antibody I-394, I-395, I-396, I-397, I-398 or I-399 fused to a human heavy chain constant region disclosed herein, optionally a constant region of any of SEQ ID NOS: 44-47, and a light chain that is a function-conservative variant of the light chain variable region of the respective I-394, I-395, I-396, I-397, I-398 or I-399 antibody fused to a human Ckappa light chain constant region.

### Production of Antibodies

Anti-CD39 antibodies may be produced by any of a variety of techniques known in the art. Typically, they are produced by immunization of a non-human animal, for example a mouse, with an immunogen comprising a CD39 polypeptide, respectively, or by screening a library of candidate binding domains with a CD39 polypeptide. The CD39 polypeptide may comprise the full length sequence of a human CD39 polypeptide, respectively, or a fragment or derivative thereof, typically an immunogenic fragment, i.e., a portion of the polypeptide comprising an epitope exposed on the surface of cells expressing a CD39 polypeptide. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human CD39 polypeptide in a lipid membrane, typically at the surface of a cell. In a specific embodiment, the immunogen comprises intact cells, particularly intact human cells, optionally treated or lysed. In another embodiment, the polypeptide is a recombinant CD39 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual, Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

For monoclonal antibodies, splenocytes are isolated from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Murine myeloma lines include, but are not limited to, those derived from MOPC-21 and MPC-11 mouse tumors available from the Salk Institute Cell Distribution Center, San Diego, U.S.A, X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to CD39 polypeptide gene products. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to CD39 polypeptides, e.g., CD39-expressing cells.

Hybridomas that are confirmed to produce a monoclonal antibody can be grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that bind(s) to the antigen of interest, i.e. CD39, particularly or essentially the same region, determinant or epitope as monoclonal antibody I-394, I-395, I-396 or I-399, can be readily determined using any one of a variety of immunological screening assays in which antibody competition can be assessed. Many such assays are routinely practiced and are well known in the art (see, e.g., U.S. Pat. No. 5,660,827, issued Aug. 26, 1997, which is specifically incorporated herein by reference).

For example, where the test antibodies to be examined are obtained from different source animals, or are even of a different Ig isotype, a simple competition assay may be employed in which the control (I-394, I-395, I-396 or I-399, for example) and test antibodies are admixed (or pre-adsorbed) and applied to a sample containing CD39 polypeptides. Protocols based upon western blotting and the use of BIACORE analysis are suitable for use in such competition studies.

In certain embodiments, one pre-mixes the control antibodies (e.g., I-394, I-395, I-396 or I-399, for example) with varying amounts of the test antibodies (e.g., about 1:10 or about 1:100) for a period of time prior to applying to the CD39 antigen sample. In other embodiments, the control and varying amounts of test antibodies can simply be admixed during exposure to the CD39 antigen sample. As long as one can distinguish bound from free antibodies (e.g., by using separation or washing techniques to eliminate unbound antibodies) and I-394 from the test antibodies (e.g., by using species-specific or isotype-specific secondary antibodies or by specifically labeling I-394, I-395, I-396 or I-399 with a detectable label) one can determine if the test antibodies reduce the binding of I-394, I-395, I-396 or I-399 to the antigens. The binding of the (labelled) control antibodies in the absence of a completely irrelevant antibody can serve as the control high value. The control low value can be obtained by incubating the labelled (I-394, I-395, I-396 or I-399) antibodies with unlabelled antibodies of exactly the same type (I-394, I-395, I-396 or I-399), where competition would occur and reduce binding of the labelled antibodies. In a test assay, a significant reduction in labelled antibody reactivity in the presence of a test antibody is indicative of a test antibody that “cross-reacts” or competes with the labelled (I-394, I-395, I-396 or I-399) antibody. Any test antibody that reduces the binding of I-394, I-395, I-396 or I-399 to CD39 antigens by at least about 50%, such as at least about 60%, or more preferably at least about 80% or 90% (e.g., about 65-100%), at any ratio of I-394, I-395, I-396 or I-399:test antibody between about 1:10 and about 1:100 can be selected. In one embodiment, such test antibody will reduce the binding of I-394, I-395, I-396 or I-399 to the CD39 antigen by at least about 90% (e.g., about 95%).

Competition can also be assessed by, for example, a flow cytometry test. In such a test, cells bearing a given CD39 polypeptide can be incubated first with I-394, for example, and then with the test antibody labelled with a fluorochrome or biotin. The antibody is said to compete with I-394 if the binding obtained upon preincubation with a saturating amount of I-394 is about 80%, preferably about 50%, about 40% or less (e.g., about 30%, 20% or 10%) of the binding (as measured by mean of fluorescence) obtained by the antibody without preincubation with I-394. Alternatively, an antibody is said to compete with I-394 if the binding obtained with a labelled I-394 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

Determination of whether an antibody binds within an epitope region can be carried out in ways known to the person skilled in the art. As one example of such mapping/characterization methods, an epitope region for an anti-CD39 antibody may be determined by epitope “foot-printing” using chemical modification of the exposed amines/carboxyls in the respective CD39 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et al., Journal of Molecular Biology, Vol. 281 (1) pp. 6₁₋₆7 (1998); and Saito and Patterson, Methods. 1996 June; 9 (3): 516-24.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801. Protease digestion techniques also can be useful in the context of epitope mapping and identification. Antigenic determinant-relevant regions/sequences can be determined by protease digestion, e.g., by using trypsin in a ratio of about 1:50 to CD39 or o/n digestion at and pH 7-8, followed by mass spectrometry (MS) analysis for peptide identification. The peptides protected from trypsin cleavage by the anti-CD39 binder can subsequently be identified by comparison of samples subjected to trypsin digestion and samples incubated with antibody and then subjected to digestion by e.g., trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the CD39 polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is replaced with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron micros-copy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fagerstam et al., Journal Of Molecular Recognition 1990; 3:208-14; Nice et al., J. Chroma-togr. 1993; 646:159-168; Leipert et al., Angew. Chem. Int. Ed. 1998; 37:3308-3311; Kroger et al., Biosensors and Bioelectronics 2002; 17:937-944.

It should also be noted that an antibody binding the same or substantially the same epitope as an antibody can be identified in one or more of the exemplary competition assays described herein.

Typically, an anti-CD39 antibody provided herein has an affinity for a respective CD39 polypeptide in the range of about 10⁴ to about 10¹¹ M⁻¹ (e.g., about 10⁸ to about 10¹⁰ M⁻¹). For example, in a particular aspect the anti-CD39 antibody that have an average disassociation constant (K_{D}) of less than 1×10⁻⁹ M with respect to CD39, respectively, as determined by, e.g., surface plasmon resonance (SPR) screening (such as by analysis with a BIAcore™ SPR analytical device). In a more particular exemplary aspect, the anti-CD39 antibodies that have a KD of about 1×10⁻⁸ M to about 1×10⁻¹⁰ M, or about 1×10⁻⁹ M to about 1×10⁻¹¹ M, for CD39, respectively. In one embodiment, binding is monovalent binding. In one embodiment, binding is bivalent binding.

Antibodies can be characterized for example by a mean KD of no more than about (i.e. better affinity than) 100, 60, 10, 5, or 1 nanomolar, preferably sub-nanomolar or optionally no more than about 500, 200, 100 or 10 picomolar. KD can be determined for example for example by immobilizing recombinantly produced human CD39 proteins on a chip surface, followed by application of the antibody to be tested in solution. In one embodiment, the method further comprises a step of selecting antibodies from (b) that are capable of competing for binding to CD39 with antibody I-394.

In one aspect of any of the embodiments, the antibodies prepared according to the present methods are monoclonal antibodies. In another aspect, the non-human animal used to produce antibodies according to the methods herein is a mammal, such as a rodent, bovine, porcine, fowl, camelid, horse, rabbit, goat, or sheep.

DNA encoding an antibody that binds an epitope present on a CD39 polypeptide is isolated from a hybridoma and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the disclosure, e.g., antibody I-394, can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody, as well as a recombinant host cell comprising (e.g., in its genome) such nucleic acid. Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific (e.g., bispecific) antibodies formed from antibody fragments. Included, inter alia, are a nanobody, domain antibody, single domain antibody or a “dAb”.

In one aspect, the agent is an antibody selected from a fully human antibody, a humanized antibody, and a chimeric antibody.

In one aspect, the agent is a fragment of an antibody comprising a constant domain selected from IgG1, IgG2, IgG3 and IgG4. In one aspect, the agent is an antibody fragment selected from a Fab fragment, a Fab′ fragment, a Fab′-SH fragment, a F(ab)2 fragment, a F(ab′)2 fragment, an Fv fragment, a Heavy chain Ig (a llama or camel Ig), a V_{HH} fragment, a single domain FV, and a single-chain antibody fragment. In one aspect, the agent is a synthetic or semisynthetic antibody-derived molecule selected from a scFV, a dsFV, a minibody, a diabody, a triabody, a kappa body, an IgNAR; and a multispecific antibody. In one aspect, the antibody is in at least partially purified form. In one aspect, the antibody is in essentially isolated form.

An anti-CD39 such as an antibody can be incorporated in a pharmaceutical formulation comprising in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g., freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through several routes of administration, for example, intravenous. Suitable antibody formulations can also be determined by examining experiences with other already developed therapeutic monoclonal antibodies. Several monoclonal antibodies have been shown to be efficient in clinical situations, such as Rituxan (Rituximab), Herceptin (Trastuzumab) Xolair (Omalizumab), Bexxar (Tositumomab), Campath (Alemtuzumab), Zevalin, Oncolym and similar formulations may be used with the antibodies of this invention.

Also provided are kits which include a pharmaceutical composition containing an anti-CD39 antibody, and an agent that induces ATP release from tumor cells, and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a solid tumor). The kit also can include a syringe.

Optionally, the kits include multiple packages of the single-dose pharmaceutical compositions each containing an effective amount of the anti-CD39 or an agent that induces ATP release from tumor cells for a single administration in accordance with the methods provided above. Instruments or devices necessary for administering the pharmaceutical composition(s) also may be included in the kits. For instance, a kit may provide one or more pre-filled syringes containing an amount of the anti-CD39 and agent that induces ATP release from tumor cells.

In one embodiment, the present invention provides a kit for treating a cancer in a human patient, the kit comprising:

(a) a dose of an anti-CD39 antibody that neutralizes the activity of sCD39, optionally wherein the antibody comprises the hypervariable region (e.g., CDR1, CDR2 and CDR3 domains) of a heavy chain variable region of antibody I-394, and the hypervariable region (e.g., CDR1, CDR2 and CDR3 domains) of a light chain variable region of antibody I-394;

(b) a dose of an agent that induces ATP release from tumor cells; and

(c) optionally, instructions for using the anti-CD39 antibody and agent that induces ATP release from tumor cells in any of the methods described herein.

### Diagnostics, Prognostics, and Treatment of Malignancies

Described herein are methods useful in the diagnosis, prognosis, monitoring, treatment and prevention of a cancer in an individual through the use of anti-CD39 antibodies to potentiate the activity of an agent that induces the extracellular release of ATP from tumor cells.

Extracellular ATP is released from tumor cells in case of stress (mechanical, hypotonic or hypoxic) or in case of cell death. Necrosis favors the passive release of ATP through the release of total cellular content, whereas apoptosis favors the release of ATP by activation of caspases 3 and 9 which cleave and activate Panexini (ATP transporter). Examples of agents that induce the extracellular release of ATP from tumor cells can include chemotherapy, radiotherapy, and, more generally, agents that induce apoptosis and thereby favor ATP release. Agents that induce the extracellular release of ATP have been shown to induce immunogenic cell death. For example, substantial ATP release can be induced by anthracyclines, oxaliplatin, cisplatin and X-rays, PARP inhibitors, taxanes, anthracyclines, DNA damaging agents, camptothecins, epothilones, mytomycins, combretastatins, vinca alkaloids, nitrogen mustards, maytansinoids, calicheamycins, duocarmycins, tubulysins, dolastatins and auristatins, enediynes, amatoxins, pyrrolobenzodiazepines, ethylenimines, radioisotopes, therapeutic proteins and peptides, and toxins or fragments thereof.

ATP can also be released through administration with depleting antibodies that bind antigens expressed at the surface of cancer cells (e.g. tumor antigen), for example antibodies that are coupled to a chemotherapeutic agent that induces ATP release, antibodies that are capable of mediating apoptosis or antibodies that direct antibody-dependent cell-mediated cytotoxicity (ADCC) toward the cancer cells (for example wherein the antibody has an Fc domain of human IgG1 isotype so as to mediate ADCC).

Anthracyclines include, for example daunorubicin, doxorubicin, epirubicin or idarubicin, optionally liposomal formulations thereof, e.g. liposomal daunorubicin such as DaunoXome™ or Vyxeos™ or CPX-351 (a combination of cytarabine and daunorubicin). Anthracyclines are widely used to treat solid and hematological malignancies, including for example acute myeloid leukemia (AML), acute lymphocytic leukemia (ALL), chronic myelogenous leukemia (CML), and Kaposi's sarcoma. Doxorubicin and its derivative, epirubicin, are used in breast cancer, childhood solid tumors, soft tissue sarcomas, and aggressive lymphomas. Daunorubicin is used to treat acute lymphoblastic or myeloblastic leukemias, and its derivative, idarubicin is used in multiple myeloma, non-Hodgkin's lymphomas, and breast cancer. Nemorubicin is used for treatment of hepatocellular carcinoma, pixantrone, used as a second-line treatment of non-Hodgkin's lymphomas. Sabarubicin is used for non-small cell lung cancer, hormone refractory metastatic prostate cancer, and platinum- or taxane-resistant ovarian cancer. Valrubicin is used for the topical treatment of bladder cancer.

Platinum agents include for example oxaliplatin, cisplatin, carboplation, nedaplatin, Phenanthriplatin, picoplatin, satraplatin.

Taxanes, include, for example Paclitaxel (Taxol) and docetaxel (Taxotere).

DNA damaging agents, include for example DNA intercalating agents, e.g. an agent that inserts itself into the DNA structure of a cell and binds to the DNA, in turn causing DNA damage (e.g. daunorubicin). Compounds include topoisomerase inhibitors, chemical compounds that block the action of topoisomerase (topoisomerase I and II. Such compounds are used for a wide range of solid tumor and hematological malignancies, notably lymphomas. Topoisomerase I inhibitors include camptothecins, for example irinotecan (approved for treatment of colon cancer), topotecan (approved for treatment of ovarian and lung cancer), camptothecin, lamellarin D, indenoisoquinoline, indimitecan. Further camtpthecins include silatecan, cositecan, exatecan, lurtotecan, gimatecan, belotecan, and rubitecan. Topoisomerase II inhibitors include for example etoposide (VP-16), teniposide, doxorubicin, daunorubicin, mitoxantrone, amsacrine, ellipticines, aurintricarboxylic acid, and HU-331, a quinolone synthesized from cannabidiol.

PARP inhibitors have been reported to tilt cell death from necrosis to caspase-independent apoptosis in cancer cells. Inhibition of PARP results in increase of intracellular ATP, which in turn is believed to result in extracellular release of ATP upon apoptosis. Poly(ADP-ribose) polymerase (PARP) family of enzymes transform NAD+ to nicotinamide and ADP-ribose to form long and branched (ADP-ribose) polymers on glutamic acid residues of a number of acceptor proteins usually associated with chromatin. PARP-1, the most abundant PARP, is a nuclear enzyme that catalyzes the formation of poly (ADP-ribose) on its target proteins using NAD+ as a substrate. PARP inhibitors typically contain as key pharmacophore benzoxazole or benzamide moieties, and various benzamide-derivatives have been reported. Example of approved PARP inhibitors that can be used in accordance with the disclosure include Olaparib (AZD-2281, Lynparza® by Astra Zeneca, approved in ovarian cancer, also effective for treatment of breast cancer, prostate cancer and colorectal cancer), Rucaparib (PF-01367338, Rubraca® by Clovis Oncology, approved in ovarian cancer), Niraparib (MK-4827, Zejula® by Tesaro, approved for epithelial, ovarian, fallopian tube, and primary peritoneal cancer). Further examples of PARP inhibitors that can be used in accordance with the disclosure include Talazoparib (BMN-673, BioMarin Pharmaceutical Inc., Pfizer) for hematological and advanced or recurrent solid tumors, Veliparib (ABT-888, developed by AbbVie) for ovarian cancer, triple-negative breast cancer and in non-small cell lung cancer (NSCLC), melanoma, CEP 9722 for non-small-cell lung cancer (NSCLC), E7016 (developed by Eisai) for melanoma, and Pamiparib (BGB-290) developed by Beigene for a variety of solid tumor malignancies.

Epothilones include for example, epothilone B and its various analogues, e.g. ixabepilone (BMS-247550) approved for treatment of breast cancer. Vinca alkaloids, include for example, vinblastine, vincristine, vindesine, and vinorelbine. Nitrogen mustards, include for example, cyclophosphamide, chlorambucil, uramustine, ifosfamide, melphalan, and bendamustine. Maytansinoids, include for example, Ansamitocin, or mertansine (DM1) or DM4 developed by Immunogen Inc.

The agents can be in any suitable configuration or formulation, including for example as free compound or as part of a conjugate, nanoparticle-formulation, encapsulated (e.g. in a liposome), in each case optionally further in a combination with additional pharmaceutically active agents. Agents can conveniently be conjugated to a targeting moiety, such as in an immunoconjugate. The terms “immunoconjugate” and “antibody conjugate” are used interchangeably and refer to an antigen binding agent, e.g., an antibody binding protein or an antibody that is conjugated to another moiety (e.g., a cytotoxic agent, a chemotherapeutic agent that induces ATP release described herein). An immunoconjugate comprising an antigen binding agent conjugated to a cytotoxic agent can also be referred to as a “antibody drug conjugate” or an “ADC”.

While the treatment regimens and methods described herein are particularly useful for the treatment of solid tumors, the treatment regimens and methods described herein can also be used for a variety of hematological cancers. The methods and compositions of the present invention are utilized for example the treatment of a variety of cancers and other proliferative diseases including, but not limited to: carcinoma, including that of the bladder, breast, colon, kidney, liver, lung, ovary, uterus, prostate, pancreas, stomach, cervix, thyroid, head and neck (head and neck squamous cell carcinoma, and skin (e.g., melanoma); hematopoietic tumors of lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma, and multiple myeloma; hematopoietic tumors of myeloid lineage, including acute and chronic myelogenous leukemias, promyelocytic leukemia, and myelodysplastic syndrome; tumors of mesenchymal origin, including fibrosarcoma and rhabdomyoscarcoma; other tumors, including melanoma, seminoma, terato-carcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, and thyroid follicular cancer.

Combination therapies for the treatment of cancer provided herein involve administration of a neutralizing anti-CD39 agent (e.g., an antibody) and an agent that induces the extracellular release of ATP, to treat subjects afflicted with cancer. In one embodiment, the invention provides an anti-CD39 antibody and a treatment (e.g., an agent) that induces the extracellular release of ATP, for use in combination, to treat subjects having a solid tumor (e.g., a solid tumor, an advanced refractory solid tumor) or subjects having a hematological tumor. In one embodiment, the provided is an anti-CD39 antibody (e.g. having the further characteristics described herein), for use in treating an individual having a cancer, where the treatment comprises administration to the individual of the anti-CD39 antibody in combination with a means for inducing apoptosis in cancerous cells (e.g., so as to induce the extracellular release of ATP in cancerous cells). In one embodiment, the provided is an anti-CD39 antibody (e.g. having the further characteristics described herein), for use in treating an individual having a cancer, where the treatment comprises administration to the individual of the anti-CD39 antibody in combination with a pharmaceutical composition comprising (a) a means (e.g. an agent or treatment) for inducing the apoptosis of cancerous cells and (b) a pharmaceutically acceptable carrier. In one embodiment, the provided is an anti-CD39 antibody (e.g. having the further characteristics described herein), for use in treating an individual having a cancer, where the treatment comprises administration to the individual of the anti-CD39 antibody in combination with a pharmaceutical composition comprising (a) a means (e.g. and agent or treatment) for inducing the extracellular release of ATP in cancerous cells and (b) a pharmaceutically acceptable carrier.

In one embodiment, the invention provides an anti-CD39 antibody for use in combination with a platinum agent (e.g. oxaliplatin, cisplatin, carboplatin, nedaplatin, phenanthriplatin, picoplatin, satraplatin, or a combination regimen comprising a platinum agent), to treat an individual having a solid tumor. In one embodiment, the solid tumor is a lung cancer, a squamous lung cancer, non-small cell lung cancer (NSCLC), an ovarian cancer, a carcinoma, a head and neck squamous cell carcinoma (HNSCC), a colorectal cancer, an urothelial cancer, a bladder cancer, a cervical cancer, a gastric cancer, an esophageal cancer or a breast cancer. In one embodiment, the combination regimen comprising a platinum agent is FOLFOX (folinic acid, 5-Fu and oxaliplatin). In one embodiment, the combination regimen comprising a platinum agent comprises carboplatin and a taxane (e.g. paclitaxel).

In one embodiment, the invention provides an anti-CD39 antibody for use in combination with a taxane agent (e.g. Paclitaxel (Taxol™) or docetaxel (Taxotere™)), to treat an individual having a solid tumor, e.g. an ovarian cancer, a breast cancer.

In one embodiment, the invention provides an anti-CD39 antibody for use in combination with gemcitabine to treat an individual having a solid tumor, e.g. an ovarian cancer.

In one embodiment, the provided is an anti-CD39 antibody for use in combination with an anthracycline agent (e.g. daunorubicin, doxorubicin, epirubicin or idarubicin), to treat an individual having a solid tumor, e.g. an ovarian cancer, a breast cancer, a non-small cell lung cancer, a colorectal cancer, a prostate cancer, a soft tissue sarcoma or a bladder cancer. In one embodiment, the invention provides an anti-CD39 antibody for use in combination with an anthracycline agent (e.g. daunorubicin, doxorubicin, epirubicin or idarubicin), to treat an individual having a hematologicall tumor, e.g. an AML, an acute lymphocytic leukemia (ALL), a chronic myelogenous leukemia (CML), a lymphoma, an acute lymphoblastic, a myeloblastic leukemia, a multiple myeloma or a non-Hodgkin's lymphoma.

In one embodiment, the invention provides an anti-CD39 antibody for use in combination with a PARP inhibitor agent (e.g. a PARP-1 inhibitor, olaparib, rucaparib, niraparib, talazoparib, veliparib, CEP 9722, E7016 or pamiparib), to treat an individual having a solid tumor, e.g. an epithelial cancer, an ovarian cancer, a breast cancer, a prostate cancer, a colorectal cancer, a fallopian tube cancer, a peritoneal cancer, a lung cancer, a non-small cell lung cancer (NSCLC) or a melanoma.

The combination treatments described herein can be particularly effective in the treatment of cancers characterized by high expression of CD39 (with or without a prior step of assessing expression or levels of CD39 in the individual), including in particular ovarian cancer, gastric cancer and esophageal cancer.

In one embodiment, provided is an anti-CD39 antibody for use in combination with a platinum agent (e.g. carboplatin), and optionally further in combination with gemcitabine, to treat an individual having an ovarian cancer.

In one embodiment, provided is an anti-CD39 antibody for use in combination with a platinum agent (e.g. oxaliplatin), optionally wherein the anti-CD39 antibody is used in combination with the FOLFOX regimen (folinic acid, 5-Fu and oxaliplatin), to treat an individual having a gastric cancer or an esophageal cancer.

In one embodiment, provided is an anti-CD39 antibody for use in combination with a platinum agent (e.g. carboplatin), and optionally further in combination with a taxane (e.g. paclitaxel), to treat an individual having a NSCLC, optionally wherein the NSCLC is a squamous cell lung cancer.

In one embodiment, provided is an anti-CD39 antibody for use in combination with FOLFOX.

In one embodiment, a solid tumor is an ovarian cancer and the individual is treated with anti-CD39 antibody in combination with oxaliplatin or carboplatin, optionally further in combination with gemcitabine. Optionally, in any embodiment herein, an ovarian cancer is a platinum-resistant ovarian cancer. An individual having a platinum-resistant ovarian cancer can for example be characterized as having cancer which has progressed, relapsed or not responded to prior treatment with a platinum-agent containing therapeutic regimen that does not include an anti-CD39 antibody. In another embodiment, an ovarian cancer can be characterized as a platinum-sensitive ovarian cancer.

The combination therapies involving an anti-CD39 antibody can be advantageously used to enhance the effect of an agent or treatment that induces the extracellular release of ATP from tumor cells and/or induces the death of tumor cells. This can be useful, for example, in an individual having a cancer (e.g., a lung cancer, ovarian cancer, colorectal cancer, gastric cancer, esophageal cancer) that is resistant to an agent or treatment that induces the extracellular release of ATP from tumor cells and/or induces the death of tumor cells. An individual having a cancer that is resistant to an agent or treatment can for example be characterized as having cancer which has progressed, relapsed or not responded to prior treatment with such agent or treatment (or a therapeutic regimen comprising such agent or treatment, wherein the regimen does not include an anti-CD39 antibody).

For example, an individual having a cancer (e.g., a breast cancer, ovarian cancer, colorectal cancer, gastric cancer, esophageal cancer) that is resistant to a member of a particular class of agent or treatment that induces the extracellular release of ATP from tumor cells (e.g. taxane, platinum agent, PARP inhibitor, or combination regimen comprising such) can be treated with an anti-CD39 antibody in combination with an agent of the said class of agent or treatment that induces the extracellular release of ATP from tumor cells. For example, in one embodiment, an anti-CD39 antibody can be used in combination with a taxane, to treat an individual having a taxane-resistant cancer. In another example, an anti-CD39 antibody can be used in combination with a platinum agent, to treat an individual having a platinum agent-resistant cancer. In another example, an anti-CD39 antibody can be used in combination with a PARP inhibitor, to treat an individual having a PARP inhibitor-resistant cancer.

In another embodiment, an individual having a cancer that is resistant to a member of a particular class of agent or treatment that induces the extracellular release of ATP from tumor cells (e.g. taxane, platinum agent, PARP inhibitor, or combination regimen comprising such) can be treated with an anti-CD39 antibody in combination with an agent of a different class of agent or treatment that induces the extracellular release of ATP from tumor cells. For example, in one embodiment, an anti-CD39 antibody can be used in combination with a taxane, to treat an individual having a platinum-resistant cancer. In another embodiment, an anti-CD39 antibody can be used in combination with a platinum agent, to treat an individual having a taxane-resistant cancer. In another embodiment, an anti-CD39 antibody can be used in combination with a PARP inhibitor, to treat an individual having a taxane-resistant and/or a platinum resistant cancer.

In one embodiment, provided is an antibody that is capable of binding and inhibiting the ATPase activity of a soluble extracellular domain human CD39 protein, for use in the treatment or prevention of a cancer in an individual, the treatment comprising:

a) determining whether the individual has a poor prognosis for response to treatment with an agent that induces the extracellular release of ATP from tumor cells, and

b) upon a determination that the individual has a poor prognosis for response to treatment with an agent that induces the extracellular release of ATP from tumor cells, administering to the individual an antibody that is capable of binding and inhibiting the ATPase activity of a human CD39 protein in the presence of exogenously added ATP. Optionally, the individual has a platinum-resistant cancer, a taxane-resistant cancer, or a PARP inhibitor-resistant cancer. Optionally, the step determining the individual has a poor prognosis for response to treatment with an agent that induces the extracellular release of ATP from tumor cells comprises assessing whether immune effector cells in a biological sample from the individual are characterized by one or markers of immune suppression and/or exhaustion, wherein the presence or and/or elevated levels of immune effector cells characterized by one or markers of immune suppression and/or exhaustion indicates a poor prognosis for response to treatment with an agent that induces the extracellular release of ATP from tumor cells.

The advantage of using an anti-CD39 antibody to enhance the effect of an agent or treatment that induces the extracellular release of ATP from tumor cells and/or induces the death of tumor cells can also be employed to achieve improved results in an individual having a cancer (e.g., a lung cancer, ovarian cancer, colorectal cancer, gastric cancer, esophageal cancer) that is sensitive (e.g. predicted or determined to be sensitive) to an agent or treatment that induces the extracellular release of ATP from tumor cells and/or induces the death of tumor cells. For example, in one embodiment, an anti-CD39 antibody can be used in combination with a taxane, to treat an individual having a taxane-sensitive cancer. In another example, an anti-CD39 antibody can be used in combination with a platinum agent, to treat an individual having a platinum agent-sensitive cancer. In another example, an anti-CD39 antibody can be used in combination with a PARP inhibitor, to treat an individual having a PARP inhibitor-sensitive cancer.

As used herein, adjunctive or combined administration (co-administration) includes simultaneous administration of the compounds in the same or different dosage form, or separate administration of the compounds (e.g., sequential administration). Thus, the anti-CD39 and the agent that induces the extracellular release of ATP can be simultaneously administered in a single formulation. Alternatively, the anti-CD39 and the agent that induces the extracellular release of ATP can be formulated for separate administration and are administered concurrently or sequentially.

A patient having a cancer can be treated with the anti-CD39 agent and the agent that induces the extracellular release of ATP with or without a prior detection step to assess tumoral ATPase activity, tumoral ATP (e.g., intratumoral ATP concentration), and/or CD39 expression on cells. Optionally, the treatment methods can comprise a step of detecting a CD39 nucleic acid or polypeptide in a biological sample of a tumor (e.g., on a tumor or tumor-infiltrating cell) from an individual.

Optionally, the treatment methods can comprise a step of detecting a CD39 nucleic acid or polypeptide in a biological sample from an individual. Examples of biological samples include any suitable biological fluid (for example serum, lymph, blood), cell sample, or tissue sample. Any determination that cells in a biological sample (e.g., cancer cells, lymphocytes, e.g., TReg cells, B cells, T cells) express CD39 at a high level, or that a high number of cells in the sample are CD39-positive, or show high intensity of staining with an anti-CD39 antibody, compared to a reference) can indicate that the individual has a cancer that may have a strong benefit from treatment with an agent that inhibits CD39 in combination with an agent that induces ATP release from tumor cells. In one embodiment, the treatment methods can comprise a step of detecting a CD39 nucleic acid or polypeptide in a biological sample of a tumor (e.g., on a tumor-infiltrating cell) from an individual.

In the treatment methods, the anti-CD39 antibody and the agent or treatment that induces the extracellular release of ATP can be administered separately, together or sequentially, or in a cocktail (where appropriate). In some embodiments, the agent or treatment that induces the extracellular release of ATP is administered prior to the administration of the anti-CD39 antibody. In preferred embodiments, the anti-CD39 antibody is administered prior to or concurrently with the administration of the agent or treatment that induces the extracellular release of ATP. In one advantageous embodiment, the anti-CD39 antibody is administered concurrently with or 0 to 15 days prior to, a course or cycle of treatment with the agent or treatment that induces the extracellular release of ATP. For example, an anti-CD39 antibody can be administered approximately 0 to 15 days prior to the administration of the agent or treatment that induces the extracellular release of ATP. In some embodiments, an anti-CD39 antibody is administered at least 1 hours, 12 hours, 24 hours or 48 hours prior to the administration of the agent or treatment that induces the extracellular release of ATP. In some embodiments, an anti-CD39 antibody is administered at least 1 hour, 12 hours, 24 hours or 48 hours prior to the administration of the agent or treatment that induces the extracellular release of ATP, but no more than 1 week prior to the administration of the agent or treatment that induces the extracellular release of ATP. In some embodiments, an anti-CD39 antibody is administered between 0 and 48 hours, or between 1 and 48 hours prior to the administration of the agent or treatment that induces the extracellular release of ATP. In some embodiments, an anti-CD39 antibody is administered from about 30 minutes to about 2 weeks, from about 30 minutes to about 1 week, from about 1 hour to about 2 weeks, from about 1 hour to about 1 week, from about 1 hour to about 2 hours, from about 2 hours to about 4 hours, from about 4 hours to about 6 hours, from about 6 hours to about 8 hours, from about 8 hours to 1 day, from about 24 or 48 hours to about 5, 6 or 7 days, from about 1 hour to about 5, 6 or 7 days, from about 1 hour to about 15 days, from about 24 or 48 hours to about 15 days, from about 3 to 7 days, or from about 1 to 5 days prior to the administration of the agent or treatment that induces the extracellular release of ATP. In some embodiments, an anti-CD39 antibody is administered concurrently with the administration of the agent or treatment that induces the extracellular release of ATP. In some advantageous embodiments, the agent or treatment that induces the extracellular release of ATP is administered at least twice within a period of 15 days or about two weeks following administration of the anti-CD39 antibody. In other embodiments, an anti-CD39 antibody is administered after the administration of agent or treatment that induces the extracellular release of ATP. In some embodiments, an anti-CD39 antibody is administered from about 30 minutes to about 2 weeks, from about 30 minutes to about 1 week, from about 1 hour to about 24, 36 or 48 hours, from about 1 hour to about 2 hours, from about 2 hours to about 4 hours, from about 4 hours to about 6 hours, from about 6 hours to about 8 hours, from about 8 hours to 1 day, or from about 1 day to about 2, 3, 4 or 5 days after the administration of the agent or treatment that induces the extracellular release of ATP.

The agent or treatment that induces the extracellular release of ATP can be administered in amounts and treatment regimens typically used for that agent or treatment in a monotherapy for the particular disease or condition being treated.

An example of a suitable amount of anti-CD39 antibody can be between 1 and 20 mg/kg body weight. In one embodiment, the amount is administered to an individual weekly, every two weeks, monthly or every two months.

In one embodiment provided is a method of treating a human individual having a cancer, comprising administering to the individual at least one administration cycle comprising an effective amount of an anti-CD39 antibody of the disclosure and an effective amount of an agent or treatment that induces the extracellular release of ATP. In one embodiment, the cycle is a period of eight weeks or less (e.g. 2 weeks, 4 weeks, 8 weeks). In one embodiment, for each of the at least one cycles, one, two, three or four doses of the anti-CD39 antibody are administered, optionally at a dose of I-20 mg/kg body weight. In one embodiment, the anti-CD39 antibody is administered by intravenous infusion. In one embodiment, for each of the at least one cycles, one, two, three or four doses of the agent or treatment that induces the extracellular release of ATP are administered.

As shown herein, the strongest anti-tumor responses were observed when repeated administration of chemotherapeutic agent in presence of saturating concentrations of anti-CD39 antibody permitted ATP accumulation and adenosine (Ado) suppression to take place during the classical two-week period required to mount an efficient anti-tumor immune response. Accordingly, in one advantageous embodiment, treatment according to the present disclosure comprises at least two successive administrations of the agent or treatment that induces the extracellular release of ATP. In one embodiment, the agent or treatment that induces the extracellular release of ATP is administered at least twice within a period of 15 days or about two weeks following administration of the anti-CD39 antibody. The anti-CD39 antibody can be administered in an amount and/or schedule such that the concentrations of anti-CD39 antibody in circulation and/or tissue of interest (e.g. tumor tissue) inhibit the ATPase activity of CD39 (e.g. at a concentration that saturates CD39 protein) at each of the two successive administrations of the agent or treatment that induces the extracellular release of ATP. For example, in one advantageous therapeutic regimen, an anti-CD39 antibody is administered concurrently with, or at least 1-48 hours before, administration of the chemotherapeutic agent inducing ATP release. In one advantageous therapeutic regimen, an anti-CD39 antibody is administered at least 1, 2, 3, 4, 5, 6 or 7 days before administration of the chemotherapeutic agent inducing ATP release.

In one embodiment, provided is antibody that is capable of binding and inhibiting the ATPase activity of human CD39 (NTPDase1) protein, for use in treating a tumor in a human individual, the treatment comprising administering to the individual an effective amount of each of an anti-CD39 antibody and an agent or treatment that induces the extracellular release of ATP from tumor cells, wherein the agent or treatment that induces the extracellular release of ATP from tumor cells is administered at least twice (e.g. in a first and a second successive administration) and wherein the anti-CD39 antibody is administered in an amount and/or schedule effective to achieve and/or to maintain a saturating concentration of anti-CD39 antibody between said two administrations of the agent or treatment that induces the extracellular release of ATP from tumor cells. The anti-CD39 antibody can be administered once or twice, for example. In one embodiment, the two administrations of the agent or treatment that induces the extracellular release of ATP from tumor cells are separated by two weeks or less (e.g. administered daily, weekly, two weekly). In one embodiment, the agent or treatment that induces the extracellular release of ATP from tumor cells is administered at least 2, 3 or 4 times during the two week period. In one embodiment, the anti-CD39 antibody can be administered in an amount that results in a concentration that is at least the minimum concentration required to substantially fully (e.g. 90%, 95%) occupy (saturate) CD39 protein. In one embodiment, the anti-CD39 antibody can be administered in an amount that results in a concentration that is at least the minimum concentration required to substantially fully (e.g. 90%, 95%) occupy (saturate) CD39 protein antibody between said two administrations of the agent or treatment that induces the extracellular release of ATP from tumor cells.

Exemplary treatment protocols for treating a human with an anti-CD39 antibody include, for example, administering to the patient an effective amount of each of anti-CD39 antibody and an agent or treatment that induces the extracellular release of ATP from tumor cells, wherein the method comprises at least one administration cycle in which at least one dose of the anti-CD39 antibody is administered and two doses of the agent or treatment that induces the extracellular release of ATP from tumor cells are administered, wherein the two doses of agent or treatment that induces the extracellular release of ATP are administered at an interval of two weeks or less. In one embodiment, the administration cycle is between 2 weeks and 8 weeks. In one embodiment, the anti-CD39 antibody can be administered in an amount and/or schedule such that the concentration of anti-CD39 antibody in circulation and/or tissue of interest (e.g. tumor tissue) inhibits the ATPase activity of CD39. Optionally, the anti-CD39 antibody is administered in an amount that provides a concentration that provides substantially full (e.g. 90%, 95%) occupation (saturation) of CD39. In one embodiment, the anti-CD39 antibody is administered concurrently with, or I-48 hours prior to, the administration of the agent or treatment that induces the extracellular release of ATP from tumor cells.

In one embodiment, provided is antibody that is capable of binding and inhibiting the ATPase activity of human CD39 (NTPDase1) protein, for use in treating a tumor in a human individual, the treatment comprising administering to the individual: (a) an anti-CD39 antibody in an amount and schedule effective to achieve and/or to maintain a saturating concentration of anti-CD39 antibody for at least one week, optionally at least two weeks (e.g. two weeks, three weeks, four weeks, or more), and (b) an agent or treatment that induces the extracellular release of ATP from tumor cells, wherein the anti-CD39 antibody is administered concurrently with, or I-48 hours prior to, the administration of the agent or treatment that induces the extracellular release of ATP from tumor cells. Optionally, the agent or treatment that induces the extracellular release of ATP from tumor cells is administered at least two times within a two week period following the administration of the anti-CD39 antibody, for example the agent or treatment that induces the extracellular release of ATP from tumor cells can be administered at least once per week.

In any embodiment, the anti-CD39 antibody can be administered in an amount that results in a concentration that is at least the minimum concentration required to substantially fully (e.g. 90%, 95%) occupy (saturate) CD39 protein antibody for one week. In one embodiment, the anti-CD39 antibody can be administered in an amount that results in a concentration that is at least the minimum concentration required to substantially fully (e.g. 90%, 95%) occupy (saturate) CD39 protein antibody for two weeks.

The anti-CD39 antibody compositions, in combination with an agent or treatment that induces the extracellular release of ATP, may optionally be combined (further combined) treatments with one or more other treatment or therapeutic agents, including agents normally utilized for the particular therapeutic purpose for which the antibody is being administered. The additional therapeutic agent will normally be administered in amounts and treatment regimens typically used for that agent in a monotherapy for the particular disease or condition being treated. In one embodiment, the additional therapeutic agent is an agent (e.g., an antibody) that inhibits CTLA-4 or the PD-1 axis (i.e. inhibits PD-1 or PD-L1). Antibodies that bind CTLA-4, PD1 or PD-L1 can be used, for example, at the exemplary the doses and/or frequencies that such agents are used as monotherapy, e.g., as described below. In one embodiment, the additional therapeutic agent is an agent (e.g., an antibody) that binds and neutralizes the 5′-ectonucleotidase activity of human CD73 protein (e.g., a soluble CD73 protein, a CD73 protein expressed by a cell). Human CD73, also known as ecto-5′-nucleotidase and as 5-prime-ribonucleotide phosphohydrolase, EC 3.1.3.5, encoded by the NT5E gene, exhibits 5′-nucleotidase, notably AMP-, NAD-, and NMN-nucleosidase, activities. CD73 catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane The amino acid sequence of Human CD73 preprotein (monomer), including a signal sequence at amino acids I-26, is shown in Genbank under accession number NP_002517, the entire disclosure of which is incorporated herein by reference, and as follows:

(SEQ ID NO: 48)
MCPRAARAPA TLLLALGAVL WPAAGAWELT
ILHTNDVHSR LEQTSEDSSK CVNASRCMGG
VARLFTKVQQ IRRAEPNVLL LDAGDQYQGT
IWFTVYKGAE VAHFMNALRY DAMALGNHEF
DNGVEGLIEP LLKEAKFPIL SANIKAKGPL
ASQISGLYLP YKVLPVGDEV VGIVGYTSKE
TPFLSNPGTN LVFEDEITAL QPEVDKLKTL
NVNKIIALGH SGFEMDKLIA QKVRGVDVVV
GGHSNTFLYT GNPPSKEVPA GKYPFIVTSD
DGRKVPVVQA YAFGKYLGYL KIEFDERGNV
ISSHGNPILL NSSIPEDPSI KADINKWRIK
LDNYSTQELG KTIVYLDGSS QSCRFRECNM
GNLICDAMIN NNLRHTDEMF WNHVSMCILN
GGGIRSPIDE RNNGTITWEN LAAVLPFGGT
FDLVQLKGST LKKAFEHSVH RYGQSTGEFL
QVGGIHVVYD LSRKPGDRVV KLDVLCTKCR
VPSYDPLKMD EVYKVILPNF LANGGDGFQM
IKDELLRHDS GDQDINVVST YISKMKVIYP
AVEGRIKFST GSHCHGSFSL IFLSLWAVIF VLYQ.

In the context herein, “inhibit”, “inhibiting”, “neutralize” or “neutralizing” when referring to the CD73 polypeptide (e.g., “neutralize CD73”, “neutralize the activity of CD73” or “neutralize the enzymatic activity of CD73”, etc.), refers to a process in which the 5′-nucleotidase (5′-ectonucleotidase) activity of CD73 is inhibited. This comprises, notably the inhibition of CD73-mediated generation of adenosine, i.e. the inhibition of CD73-mediated catabolism of AMP to adenosine. This can be measured for example in a cell-free assay that measures the capacity of a test compound to inhibit the conversion of AMP to adenosine, either directly or indirectly. In one embodiment, an antibody preparation causes at least a 50% decrease in the conversion of AMP to adenosine, at least a 70% decrease in the conversion of AMP to adenosine, or at least an 80% decrease in the conversion of AMP to adenosine, referring, for example, to the assays described herein.

### EXAMPLES

### Methods

### Generation of CD39 Mutants

CD39 mutants were generated by PCR. The sequences amplified were run on agarose gel and purified using the Macherey Nagel PCR Clean-Up Gel Extraction kit (reference 740609). The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a C002 incubator for 48 hours prior to testing for transgene expression. Mutants were transfected in Hek-293T cells, as shown in the table below. The targeted amino acid mutations in the table 1 below are shown using numbering of SEQ ID NO: 1.

TABLE 1

| Mutant | Substitutions | | | | | |
|---|---|---|---|---|---|---|
| 1 | V77G | H79Q | Q444K | G445D | | |
| 2A | V81S | E82A | R111A | V115A | | |
| 2B | E110A | R113T | E114A | | | |
| 3 | R118A | S119A | Q120K | Q122H | E123A | |
| 4 | D150A | E153S | R154A | S157K | N158A | L278F |
| 5 | Q96A | N99A | E143A | R147E | | |
| 6 | K188R | Replacement of the residues 190 to 207 by KTPGGS | | | | |
| 7 | A273S | N275A | I277S | R279A | | |
| 8 | S294A | K298G | K303A | E306A | T308K | Q312A |
| 9 | K288E | K289A | V290A | E315R | | |
| 10A | Q354A | D356S | E435A | H436Q | | |
| 10B | H428A | T430A | A431D | D432A | | |
| 11 | N371K | L372K | E375A | K376G | Insertion377V | V377S |
| 12 | K388N | Q392K | P393S | E396A | | |
| 13 | A402P | G403A | K405A | E406A | | |
| 15 | K87A | E100A | D107A | | | |
| 16 | Q323A | Q324A | Q327A | E331 K | | |
| 17 | N334A | S336A | Y337G | N346A | | |
| 18 | Q228A | I230S | D234A | Q238A | | |
| 19 | R138A | M139A | E142K | | | |

Cloning, production and purification of soluble huCD39 Molecular Biology

The huCD39 protein was cloned from human PBMC cDNA using the following primers ACAGGCGTGCATTCGCAGAACAAAGCATTGCCAG (SEQ ID NO: 35) (Forward), and CCGCCCCGACTCTAGATCACTTGTCATCGTCATCTTTGTAATCGA CATAGGTGGAGTGGGAGAG (SEQ ID NO: 36) (Reverse). The purified PCR product was then cloned into an expression vector using the InFusion cloning system. A M2 tag (FLAG tag, underlined in SEQ ID NO: 39) was added in the C-terminal part of the protein for the purification step; it will be appreciated that a CD39 extracellular domain protein (e.g., of SEQ ID NO: 39) can in any embodiment optionally be specified to lack the M2 tag.

Expression and Purification of the huCD39 Proteins

After validation of the sequence cloned, CHO cells were nucleofected and the producing pool was then sub-cloned to obtain a cell clone producing the huCD39 protein. Supernatant from the huCD39 clone grown in roller was harvested and purified using M2 chromatography column and eluted using the M2 peptide. The purified proteins were then loaded onto a S200 size exclusion chromatography column. The purified protein corresponding to a monomer was formulated in a TBS PH7.5 buffer. The amino acid sequence of the CD39-M2 extracellular domain recombinant protein without M2 tag was as follows:

(SEQ ID NO: 2)
TQNKALPENVKYGIVLDAGSSHTSLYIYKWPAEKENDTGVVHQVEECRVK
GPGISKFVQKVNEIGIYLTDCMERAREVIPRSQHQETPVYLGATAGMRLL
RMESEELADRVLDVVERSLSNYPFDFQGARIITGQEEGAYGWITINYLLG
KFSQKTRWFSIVPYETNNQETFGALDLGGASTQVTFVPQNQTIESPDNAL
QFRLYGKDYNVYTHSFLCYGKDQALWQKLAKDIQVASNEILRDPCFHPGY
KKVVNVSDLYKTPCTKRFEMTLPFQQFEIQGIGNYQQCHQSILELFNTSY
CPYSQCAFNGIFLPPLQGDFGAFSAFYFVMKFLNLTSEKVSQEKVTEMMK
KFCAQPWEEIKTSYAGVKEKYLSEYCFSGTYILSLLLQGYHFTADSWEHI
HFIGKIQGSDAGWTLGYMLNLTNMIPAEQPLSTPLSHSTYV.

The final amino acid sequence of the CD39-M2 extracellular domain recombinant protein with the M2 tag was as follows:

(SEQ ID NO: 39)
TQNKALPENVKYGIVLDAGSSHTSLYIYKWPAEKENDTGVVHQVEECRVK
GPGISKFVQKVNEIGIYLTDCMERAREVIPRSQHQETPVYLGATAGMRLL
RMESEELADRVLDVVERSLSNYPFDFQGARIITGQEEGAYGWITINYLLG
KFSQKTRWFSIVPYETNNQETFGALDLGGASTQVTFVPQNQTIESPDNAL
QFRLYGKDYNVYTHSFLCYGKDQALWQKLAKDIQVASNEILRDPCFHPGY
KKVVNVSDLYKTPCTKRFEMTLPFQQFEIQGIGNYQQCHQSILELFNTSY
CPYSQCAFNGIFLPPLQGDFGAFSAFYFVMKFLNLTSEKVSQEKVTEMMK
KFCAQPWEEIKTSYAGVKEKYLSEYCFSGTYILSLLLQGYHFTADSWEHI
HFIGKIQGSDAGWTLGYMLNLTNMIPAEQPLSTPLSHSTYVDYKDDDDK.

### Inhibition of the Enzymatic Activity of Soluble CD39

The inhibition by antibodies of the enzymatic activity of soluble CD39 protein produced was evaluated using Cell Titer Glo™ (Promega, reference G7571) that allows assessment of ATP hydrolysis through use of a reagent that generates a luminescent signal proportional to the amount of ATP present. In this way, inhibition of the soluble-CD39-meidatd ATP hydrolysis can be assessed. Briefly, dose ranges of anti-CD39 antibodies from 100 μg/ml to 6×10⁻³ μg/ml were incubated with 400 ng/ml of soluble recombinant human CD39 protein having the amino acid sequence described in the Methods section (SEQ ID NO: 39), for 1 h at 37° C. 20 μM ATP was added to the plates for 30 additional minutes at 37° C. before addition of CTG (Cell Titer Glo) reagent. Emitted light was quantified using an Enspire™ luminometer after a short incubation period of 5 min in the dark. Anti-CD39 antibody efficacy was determined by comparing emitted light in presence of antibody with ATP alone (maximal light emission) and ATP together with soluble CD39 protein (minimal light emission).

### Inhibition of the Enzymatic Activity of Cellular CD39

The inhibition of the CD39 enzymatic activity in CD39-expressing cells by antibodies was evaluated using Cell Titer Glo™ (Promega, reference G7571) that allows assessment of ATP hydrolysis through use of a reagent that generates a luminescent signal proportional to the amount of ATP present. The assay was thus designed to permit assessment of the inhibition of ATP hydrolyzed by CD39 in the cell culture supernatant. Briefly, 5×10⁴ Ramos human lymphoma cells, 5×10³ human CD39-, cynomolgus CD39- and mouse CD39-expressing CHO cells, were incubated 1 hour at 37° C. with anti-CD39 antibodies from 30 μg/ml to 5×10⁻⁴ μg/ml. Cells were then incubated with 20 μM ATP for 1 additional hour at 37° C. Plates were centrifuged for 2 min at 400 g and 50 μl cell supernatant are transferred in a luminescence microplate (white wells). 50 μl CellTiter-Glo® Reagent (CTG) was added to the supernatant and emitted light was quantified after a 5 min incubation in the dark using a Enspire™ luminometer. Anti-CD39 antibody efficacy was determined by comparing emitted light in presence of antibody with ATP alone (maximal light emission) and ATP together with cells (minimal light emission).

### Generation of Antibodies: Immunization and Screening in Mice

To obtain anti-human CD39 antibodies, Balb/c mice were immunized with the recombinant human CD39-M2 extracellular domain recombinant protein described above. Mice received one primo-immunization with an emulsion of 50 μg CD39 protein and Complete Freund Adjuvant, intraperitoneally, a 2nd immunization with an emulsion of 50 μg CD39 protein and Incomplete Freund Adjuvant, intraperitoneally, and finally a boost with 10 μg CD39 protein, intravenously. Immune spleen cells were fused 3 days after the boost with X63.Ag8.653 immortalized B cells, and cultured in the presence of irradiated spleen cells. Hydridomas were plated in semi-solid methylcellulose-containing medium and growing clones were picked using a clonepix 2 apparatus (Molecular Devices).

### Example 1: Epitope Mapping of Known Neutralizing CD39 mAbs

In order to gain insight into how antibodies that are able to inhibit the enzymatic (ATPase) activity of cellular CD39, we investigated the epitopes bound by antibodies that have been reported to inhibit the ATPase activity of CD39 in cellular assays: BY40 disclosed in PCT publication no. WO2009/095478.

In order to define the epitopes of anti-CD39 antibodies, we designed CD39 mutants defined by substitutions of amino acids exposed at the molecular surface over the surface of CD39. Mutants were transfected in Hek-293T cells, as shown in Table 1, using numbering of SEQ ID NO: 1.

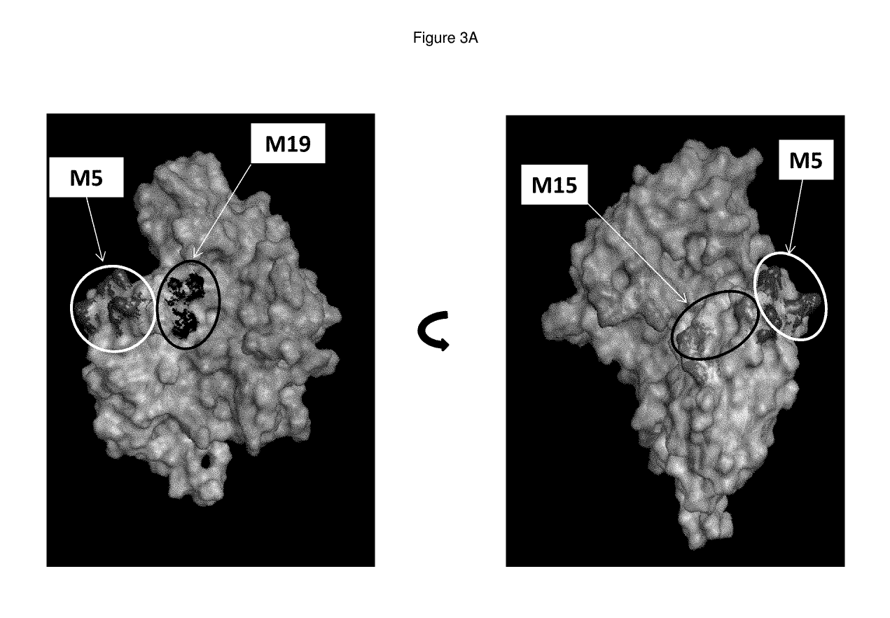

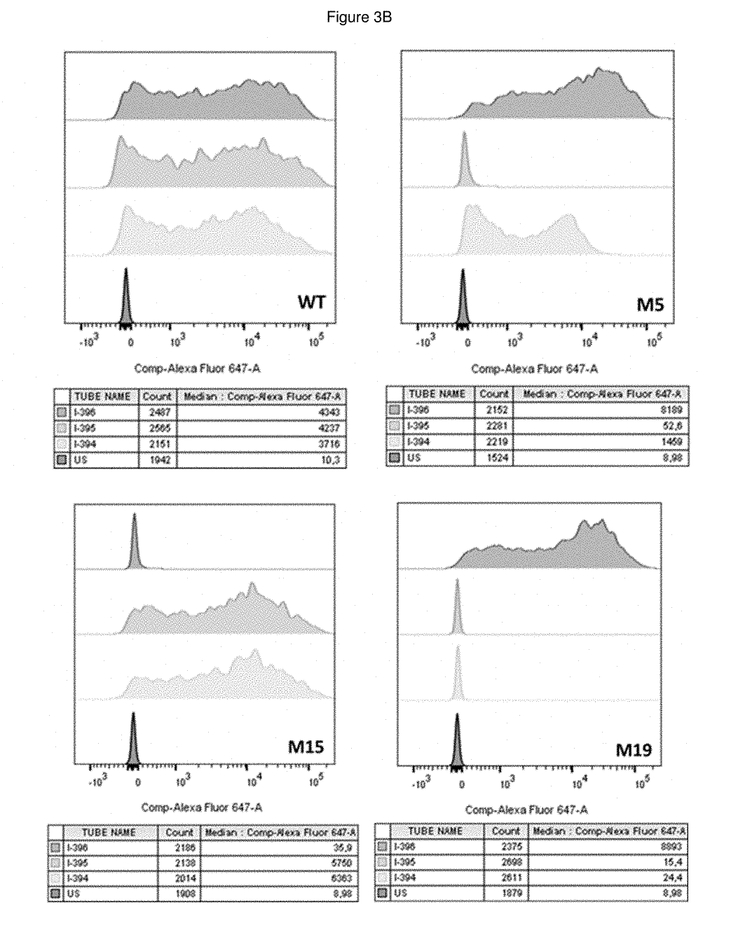

Dose-ranges of I-394 (10-2.5-0.625-0.1563-0.0391-0.0098-0.0024-0.0006 μg/ml) are tested on the 20 generated mutants by flow cytometry. BY40 antibodies both had complete loss of binding to cells expressing mutant 5 of CD39, without loss of binding to any other mutant. Mutant 5 contains amino acid substitutions at residues Q96, N99, E143 and R147. The position of Mutant 5 on the surface of CD39 is shown in FIG. 3A.

### Example 2: Known Neutralizing CD39 mAbs are Unable to Inhibit the ATPase Activity of Recombinant Soluble CD39 Protein

The two antibodies that have been reported to inhibit the ATPase activity of CD39 in cellular assays (BY40 and BY12) were assessed to determine whether are able to inhibit the ATPase activity of recombinant soluble CD39 protein. The inhibition by antibodies of the enzymatic activity of soluble CD39 protein produced as described above was evaluated using Cell Titer Glo™ (Promega, reference G7571). The inhibition by antibodies of the enzymatic activity of cellular CD39 protein was evaluated as indicated above.

As expected, BY40 inhibited the ATPase activity of CD39 protein in cells. However, BY40 was unable to inhibit the enzymatic activity of soluble CD39 protein. FIG. 2B shows a comparison of BY40 with the new antibodies identified herein.

### Example 3: Screening for New mAbs to Block sCD39 Activity

A series of immunizations were carried out in order to seek antibodies that neutralize the ATPase activity of sCD39. To obtain anti-human CD39 antibodies, animals were immunized with the recombinant human CD39-M2 extracellular domain recombinant protein described above. In total, 15 series of immunizations were carried out using different protocols and in different animals. Included were different mice strains, rats and rabbits.

In initial immunization protocols, the primary screen involved testing supernatant (SN) of growing clones by flow cytometry using wild type CHO and CHO expressing huCD39 cell lines. Cells were stained with 0.1 μM and 0.005 μM CFSE, respectively. For the flow cytometry screening, all cells were equally mixed and the presence of reacting antibodies in supernatants was revealed by Goat anti-mouse polyclonal antibody (pAb) labeled with APC. For antibodies that bound huCD39, supernatants were then screened for inhibition of the enzymatic activity of soluble CD39 using the screening assay developed and described above (Methods).

Results showed that while numerous specific CD39-binding antibodies could be obtained, none of the antibodies from any of these immunizations showed any inhibition of the enzymatic activity of soluble CD39. One possibility is that dominant epitopes on CD39 do not include any epitopes suitably positioned at or near that catalytic site of CD39. In view of the few antibodies available that inhibit cellular CD39 and the known difficulties in inhibiting the catalytic sites of enzymes using antibodies, the absence of antibodies that neutralize sCD39 may indicate that it is not possible to obtain antibodies that inhibit soluble (extracellular domain) CD39. Other possibilities relate to non-functional screening assays and/or improperly folded or functioning soluble CD39 protein, particularly since the lack of any antibody that can inhibit soluble CD39 hampers validation of sCD39 blockade assays.

In view of the absence of antibodies able to inhibit soluble CD39, a further immunization was carried out with a screening protocol designed to favor the generation of antibodies that bind the active site of CD39 as identified by the epitope of antibody BY40. Briefly, the primary screen involved testing supernatant (SN) of growing clones by flow cytometry using wild type CHO and CHO expressing huCD39 cell lines, as in the preceding immunizations, followed by screening for loss of binding Hek-293T cells expressing CD39 mutant 5, compared to wild-type CD39, as shown in Table 1. Mutant 5 has substitutions at residues Q96, N99, E143 and R147. However, again results showed that while numerous specific CD39-binding antibodies could be obtained that showed loss of binding to mutant 5, none of the antibodies from any of the initial immunizations showed any inhibition of the enzymatic activity of soluble CD39.

### Example 4: Identification of a First Antibody that Inhibits sCD39 Activity as Part of an Epitope-Directed Screen

We sought to identify anti-CD39 antibodies that do not bind the Q96, N99, E143 and R147 region (defined by mutant 5) in order to have antibodies that do not compete with BY40-like antibodies. Such antibodies which need not have any ability to block the ATPase activity of CD39 can be useful for pharmacology studies of antibodies that inhibit cellular CD39 which bind to the BY40 binding site, e.g., to detect and quantify free CD39 proteins on cells in the presence of BY40 or BY40-like antibodies that inhibit cellular CD39.

Starting from the results of the immunization of Example 3 in which hybridomas were screened for loss of binding to CD39 mutant 5, a hybridoma was selected that was not among those that showed loss of binding to CD39 mutant 5. This hybridoma (I-394) was among the broader pool due to inconclusive data indicating possible partial decrease in binding to mutant 5, but did not lose binding to mutant 5 and was therefore not initially retained.

In the context of ongoing screening of supernatants from further immunizations for inhibition of the enzymatic activity of soluble CD39, the antibody I-394 that had been cloned and produced was included as a control. Surprisingly, despite antibody I-394 not being among the clones retained in the epitope-directed screen, this antibody showed strong inhibition of the enzymatic activity of soluble CD39 in the assay described above (Methods).

I-394 was produced with human constant regions of IgG1 isotype, with a modified Fc domain having the mutations L234A/L235E/G237A/A330S/P331S (Kabat EU numbering) which results in lack of binding to human Fcγ receptors CD16A, CD16B, CD32A, CD32B and CD64, Briefly, the VH and Vk sequences of the I-394 antibody (the VH and Vk variable regions shown in SEQ ID NOS: 3 and 4, respectively) were cloned into expression vectors containing the huIgG1 constant domains harboring the aforementioned mutations and the huCk constant domain respectively. The two obtained vectors were co-transfected into the CHO cell line. The established pool of cell was used to produce the antibody in the CHO medium. The antibody was then purified using protein A. The amino acid sequences of the respective heavy and light chain variable domains of I-394 are shown below (Kabat CDRs underlined).

I-394 heavy chain variable domain sequence:
(SEQ ID NO: 3)
EVQLQQSGPELVKPGASVKMSCKASGYTFTDYNMHWVKQSHGRTLEWIGY
IVPLNGGSTFNQKFKGRATLTVNTSSRTAYMELRSLTSEDSAAYYCARGG
TRFAYWGQGTLVTVSA.
I-394 light chain variable domain sequence:
(SEQ ID NO: 4)
DIVLTQSPASLAVSLGQRATISCRASESVDNFGVSFMYWFQQKPGQPPNL
LIYGASNQGSGVPARFRGSGSGTDFSLNIHPMEADDTAMYFCQQTKEVPY
TFGGGTKLEIK.

The heavy and light chain sequences of I-394 with human IgG1 constant regions, with L234A/L235E/G237A/A330S/P331S substitutions (retaining N297-linked glycosylation) are shown below:

I-394 heavy chain sequence:
(SEQ ID NO: 37)
EVQLQQSGPELVKPGASVKMSCKASGYTFTDYNMHWVKQSHGRTLEWIGY
IVPLNGGSTFNQKFKGRATLTVNTSSRTAYMELRSLTSEDSAAYYCARGG
TRFAYWGQGTLVTVSAASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPEAEGAPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPSSIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.
I-394 light chain sequence:
(SEQ ID NO: 38)
DIVLTQSPASLAVSLGQRATISCRASESVDNEGVSEMYWFQQKPGQPPNL
LIYGASNQGSGVPARFRGSGSGTDFSLNIHPMEADDTAMYFCQQTKEVPY
TFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

Antibody I-394 was then tested for loss of binding to CD39 mutants defined by substitutions of amino acids exposed at the molecular surface over the surface of CD39. Mutants were transfected in Hek-293T cells, as shown in the table 1, using numbering of SEQ ID NO: 1. Dose-ranges of antibodies I-394 were tested on the 20 mutants by flow cytometry. As shown in FIG. 3B, I-394 showed complete loss of binding to cells expressing mutant 19 of CD39. Mutant 19 includes substitutions at residues R138, M139 and E142. The core epitope of I-394 thus includes one or more (or all of) residues R138, M139 and E142.

Unlike prior antibody BY40 which loses binding to mutant 5 and has the ability to inhibit cellular CD39 but not soluble CD39, antibody I-394 loses binding to the adjacent mutant 19, with strongly reduced binding to mutant 5 (but with some residual binding to mutant 5). Interestingly, the residues of mutant 19 are in close proximity or adjacent to those of residue 5, such that I-394 may represent a shift in epitope compared to BY40. Antibody I-394 thus presents a valuable new epitope for anti-CD39 antibodies that permits inhibition of the ATPase activity of soluble CD39 protein. It also provides a specific positive control that permits the validation and testing of screening assays for detecting further antibodies that neutralize the ATPase activity of soluble CD39 protein.

### Example 5: A Non-Epitope Directed Screen for sCD39-Neutralizating mAbs

Based on the results for Example 4 indicating the antibody-mediated inhibition of soluble CD39 is possible, fusions from the different immunizations using different protocols from Example 3 were revisited in order to seek antibodies that neutralize the ATPase activity of sCD39.

Different approaches for screening for ATPase inhibition were then evaluated. In one experiment, I-394 antibody was used to spike supernatants from hybridomas of an immunization of Example 3 that were found negative for ability to inhibit the ATPase activity of soluble CD39. This addition of I-394 to supernatant did not restore the ability of negative supernatants to inhibit ATPase activity of CD39. Antibody I-394 was then purified from the negative supernatant using Protein A coated beads, and we observed the purified I-394 was again able to inhibit of ATPase activity was restored.

In view of the foregoing results, new immunization and screening protocols were developed in which growing clones from new and past immunizations were screened by flow cytometry using wild type CHO and CHO expressing huCD39 cell lines without assessment of inhibition of soluble CD39 or cellular CD39 ATPase activity, and without screening bias for epitopes. While data regarding loss of binding to mutant 5 or 19 was available for some hybridomas, such data was not used for clone selection but only retained for purposes of rescuing hybridomas for cloning in the event of negative results in the ATPase blocking assay. Hybridomas that bind CD39 were selected and cloned, and then purified using Protein A according to the following protocol:
Add to 300 μl of hybridomas supernatant 10 μl of protein A beads
Add NaCl to be at a final concentration of 1.5M
Rotate the tubes for 3-4 h at 4° C.
Centrifuge 1 min at 1500 rpm
Eliminate the supernatant and perform three washes with 1 ml of TBS
Eliminate all the TBS after the third wash
Add 50 μl of Citrate 0.1M pH3, homogenize and incubate at RT for 5 min
Centrifuge the beads for 1 min at 1500 rpm
Harvest the 50 μl of elution and add rapidly 450 μl of TBS and store at 4° C.

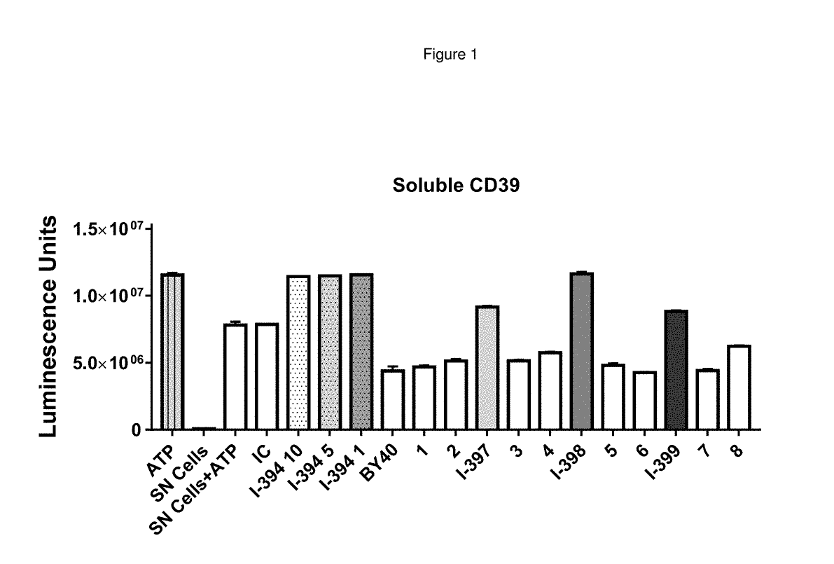

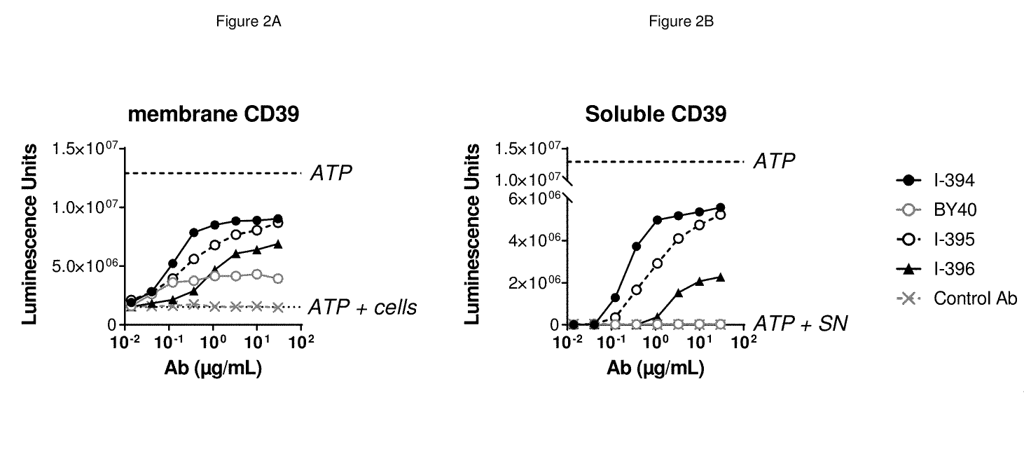

The antibodies obtained were then screened in a comparative assay for the ability to inhibit the ATPase activity of CD39 to a similar degree as I-394. Assays used for inhibition of the enzymatic activity of soluble and cellular CD39 were as described above (Methods). Surprisingly, among the exemplary antibodies produced in this way, several showed inhibition of soluble CD39 (as well as inhibition of cellular CD39). FIG. 1 shows a representative screening result, showing antibodies I-397, I-398 and I-399 compared to positive control I-394 antibody. Similarly, antibodies I-395 and I-396 from different immunization inhibited the enzymatic activity of soluble CD39 protein. FIGS. 2A and 2B shows results for antibodies I-395 and I-396 for which greater quantities of antibodies were available for additional experiments for both soluble and cellular CD39 neutralization. FIG. 2A shows that antibodies I-395 and I-396 both inhibit cell-membrane bound CD39 in comparison to BY40 and I-394 antibodies, with both I-394 and I-395 showing greater potency and maximal inhibition of cellular CD39 compared to BY40. FIG. 2B shows that antibodies I-395 and I-396 both inhibit soluble CD39 in comparison to BY40 and I-394 antibodies. While BY40 does not inhibit soluble CD39 at any concentration, I-394, I-395 and I-396 all inhibit soluble CD39 with I-394 showing the greatest potency, followed by I-395 and then I-396 with lower potency.

The results obtained raise the possibility that factor(s) in hybridoma supernatants are rapidly hydrolyzing ATP in both cell culture and in the soluble CD39 assay, such that no signal for ATP is detected in screening of antibodies using conventional methods. The soluble factor may be CD39 or some other enzyme, for example produced by the fusion partner.

Antibodies were then cloned, with modification to have a human constant regions with an IgG1 Fc domain having the mutations L234A/L235E/G237A/A330S/P331S (Kabat EU numbering) which results in lack of binding to human Fcγ receptors CD16A, CD16B, CD32A, CD32B and CD64, in the same way as shown herein for I-394. The resulting antibodies can then be subjected to titrations and then more detailed activity assessment as shown in Example 7-9 (titration, inhibition of ATPase activity) to assess EC₅₀ and IC₅₀ determinations to rank antibodies according to potency.

### Example 6: Epitope Mapping of sCD39 Neutralizing mAbs

As shown in Example 4, I-394 showed complete loss of binding to cells expressing mutant 19 of CD39, but did not lose binding to mutant 5. In order to define the epitopes of the further anti-CD39 antibodies of Example 5, they were tested for loss of binding to the panel of CD39 mutants as described in Example 1 and Table 1. Mutants were transfected in Hek-293T cells, as shown in the table 1, using numbering of SEQ ID NO: 1. Dose-ranges of test antibodies (10-2.5-0.625-0.1563-0.0391-0.0098-0.0024-0.0006 μg/ml) are tested on the 20 generated mutants by flow cytometry.

Results showed that the antibodies selected in Example 5 for ability to inhibit soluble CD39 represented several different epitopes. Among the antibodies that showed inhibition of soluble extracellular CD39 in Example 5, antibody I-395 is an example of an antibody that displayed loss of binding to mutant 5 having substitutions at residues Q96, N99, E143 and R147, and also loss of binding to mutant 19 having substitutions at residues R138, M139 and E142. Mutant 19 includes substitutions at residues R138, M139 and E142. The core epitope on CD39 of I-395 thus comprises one, two, three or four of residues Q96, N99, E143 and R147 as well as one, two or three of residues R138, M139 and E142.

Antibody I-398 on the other hand, is an example of an antibody that displayed loss of binding to mutant 19 having substitutions at residues R138, M139 and E142, but does not have decreased or loss of binding to mutant 5 having substitutions at residues Q96, N99, E143 and R147.

Other antibodies that showed inhibition of soluble extracellular CD39 in Example 5 had very different epitopes and did not show loss of binding to either of mutants 5 or 19, suggesting that soluble CD39 can also be inhibited by binding to other sites on sCD39. For some antibodies, loss of binding to one of the 20 mutants of Table 1 permitted the localization of binding site on CD39, while for others the binding site remained to be determined as they did not lose binding to any of the 20 mutants. Among the antibodies showing inhibition of ATPase activity of soluble CD39 in Example 5, antibody I-396 showed loss of binding to mutant 15 having substitutions K87A, E100A and D107A, without loss of binding to any of the other 20 mutants. The core epitope on CD39 of this antibody thus comprises one or more (or all of) residues K87, E100 and D107. Antibody I-399 showed loss of binding to mutant 11 having substitutions N371K, L372K, E375A, K376G, V377A and an insertion of a valine between K376 and V377 (referred to in Table 1 as “insertion 377V”), without loss of binding to any of the other 20 mutants. The core epitope on CD39 of this antibody thus comprises one or more (or all of) residues N371, L372, E375, K376 and V377. FIG. 3A shows the position of residues mutated in mutants 5 (M5), 15 (M15) and 19 (M19) on the surface of the CD39 protein. FIG. 3B shows results of binding to mutants 5, 15 and 19 for different antibodies.

The results thus show that antibodies that inhibit soluble CD39 can be obtained against different epitopes. The epitopes include epitopes defined by one or more residues of mutant 19 which are located adjacent to the binding site of the BY40 or BY40-like antibodies that inhibit only cellular CD39 but not soluble CD39 (which lose binding to mutant 5), epitopes that are defined by one or more residues of mutant 19 but also partly by mutant 5, indicating possibly a smaller shift compared to BY40 or BY40-like antibodies, epitopes defined by one or more residues of mutant 19 and not by residues of mutant 5, as well as other epitopes such as those defined by one or more residues of mutant 11 or one or more residues of mutant 15, or further by other antibodies that do not have any reduced binding to any of mutants 5, 15 or 19 for which localization of epitopes remain to be determined.

### Example 7: Antibody Titration on CD39 Expressing Cells by Flow Cytometry

Antibody I-394 was tested in two repeated experiments for binding to CHO cells expressing human CD39, CHO cells expressing cynomolgus (macaca fascicularis) CD39, CHO cells expressing murine CD39, and human Ramos lymphoma cells (ATCC™, reference CRL-1596). Cells were incubated with various concentration of unlabeled anti-CD39 antibody from 30 μg/ml to 5×10⁻⁴ μg/ml, for 30 minutes at 4° C. After washes, cells were incubated with Goat anti-mouse H+L labeled secondary antibody for 30 min at 4° C.

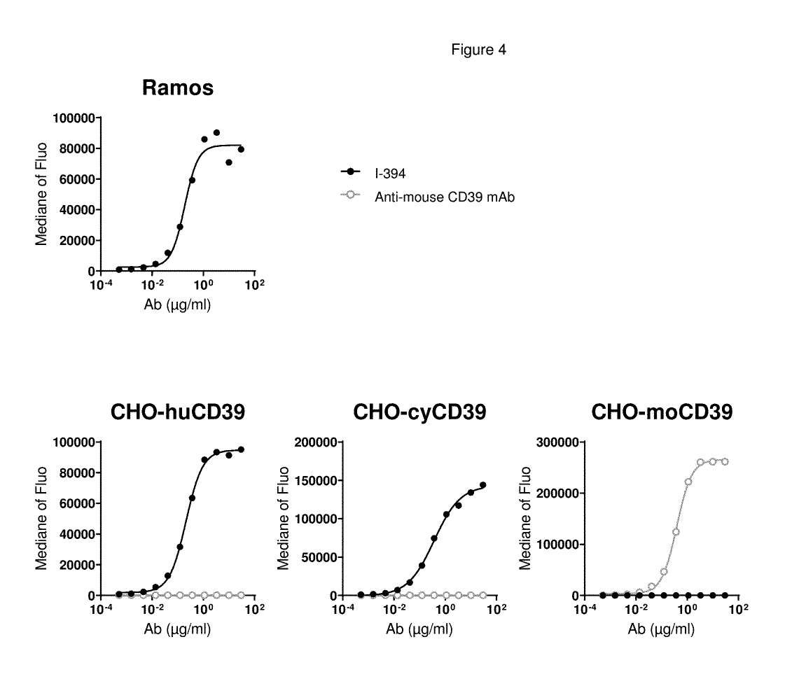

Results are shown in FIG. 4. Antibody I-394 bound to cells expressing human CD39 (CHO-huCD39), cells expressing cynomolgus CD39 (CHO-cyCD39) and to Ramos lymphoma cells, but not to cells expressing murine CD39 (CHO-moCD39). I-394 bound to Ramos cells with EC₅₀ values of 0.16 μg/ml and 0.19 μg/ml in the respective first and second set of experiments. Several other anti-CD39 antibodies showed comparable EC₅₀ values for binding to Ramos cells.

### Example 8: IC50 Determination for Inhibition of Cellular ATPase Activity

The inhibition by antibody I-394 of the ATPase activity of CD39 in CD39-expressing cells was evaluated using the assay used for inhibition of the enzymatic activity of cellular CD39 as described above (Methods).

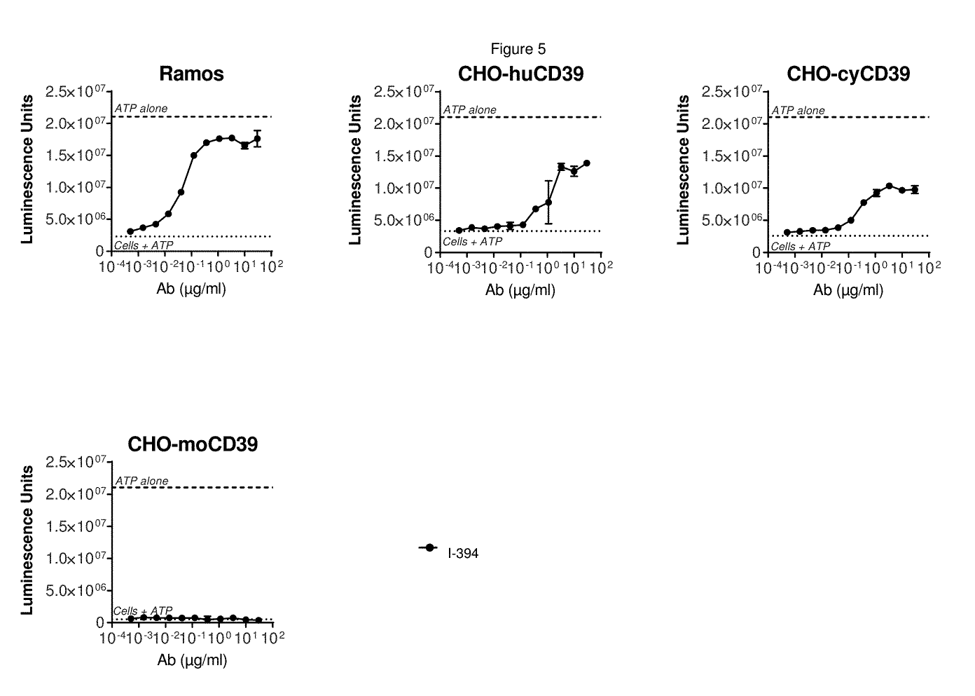

Results are shown in FIG. 5. I-394 is highly potent at blocking CD39 enzymatic activity in tumor (Ramos) cells, with greater potency compared to all other antibodies tested. I-394 also blocks CD39 enzymatic activity in cells expressing human CD39 (CHO-huCD39), and in cells expressing cynomolgus CD39 (CHO-cyCD39). Cells expressing murine CD39 (CHO-moCD39) are shown as a negative control. The calculated IC₅₀ (inhibition of 50% of the enzymatic activity of CD39 expressed by 50,000 Ramos cells) is 0.05 μg/ml. The maximum inhibition achieved is 81.6%. Isotype control had no effect.

### Example 9: IC50 Determination for Inhibition of the ATPase Activity of Recombinant Soluble CD39 Protein

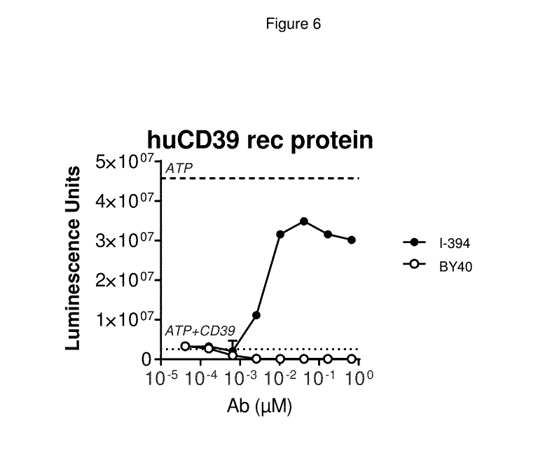

The inhibition by antibody I-394 of the ATPase activity of soluble CD39 protein was evaluated using the assays used for inhibition of the enzymatic activity of soluble CD39 as described above (Methods). Results are shown in FIG. 6. I-394 inhibits the enzymatic activity of soluble CD39 protein. Antibody BY40 in comparison did not inhibit the enzymatic activity of soluble CD39 protein. The calculated IC₅₀ is 0.003 μg/ml. The maximum inhibition achieved is 74.9%.

### Example 10: ELISA Titration on CD39-L1, L2, L3, L4 Isoforms

Antibody I-394 was tested for binding to recombinant human CD39 isoforms (Rec-huCD39 isoforms) having amino acid sequences shown below were coated in 96-well plate in PBS 1× at 500 ng/ml or 1 μg/ml at 4° C. overnight. Wells were washed in TBS Tween 20, and further saturated 2H at RT in TBS Blocking buffer. Dose range concentration of primary antibody was incubated in TBS blocking buffer for 2 h at RT. Wells were washed in TBS Tween 20. Secondary Antibody (GAM-HRP or GAH-HRP in TBS blocking buffer) was incubated for 1H at RT, and was revealed with TMB. Optical density was measured on Enspire™ at OD=450.

Amino Acid Sequence of the Cone huCD39 (Vascular Isoform):

| Human CD39-L1, also known as NTPDase2 or ENTPD2: | |
|---|---|
| (SEQ ID NO: 40) | |
| 1 | MAGKVRSLLP PLLLAAAGLA GLLLLCVPTR DVREPPALKY GIVLDAGSSH TSMFIYKWPA |
| 61 | DKENDTGIVG QHSSCDVPGG GISSYADNPS GASQSLVGCL EQALQDVPKE RHAGTPLYLG |
| 121 | ATAGMRLLNL TNPEASTSVL MAVTHTLTQY PFDFRGARIL SGQEEGVFGW VTANYLLENF |
| 181 | IKYGWVGRWF RPRKGTLGAM DLGGASTQIT FETTSPAEDR ASEVQLHLYG QHYRVYTHSF |
| 241 | LCYGRDQVLQ RLLASALQTH GFHPCWPRGF STQVLLGDVY QSPCTMAQRP QNFNSSARVS |
| 301 | LSGSSDPHLC RDLVSGLFSF SSCPFSRCSF NGVFQPPVAG NFVAFSAFFY TVDFLRTSMG |
| 361 | LPVATLQQLE AAAVNVCNQT WAQQLLSRGY GFDERAFGGV IFQKKAADTA VGWALGYMLN |
| 421 | LTNLIPADPP GLRKGTDFSS WVVLLLLFAS ALLAALVLLL RQVHSAKLPS TI. |
| Human CD39-L2, also known as NTPDase6 or ENTPD6: | |
| (SEQ ID NO: 41) | |
| 1 | MKKGIRYETS RKTSYIFQQP QHGPWQTRMR KISNHGSLRV AKVAYPLGLC VGVFIYVAYI |
| 61 | KWHRATATQA FFSITRAAPG ARWGQQAHSP LGTAADGHEV FYGIMFDAGS TGTRVHVFQF |
| 121 | TRPPRETPTL THETFKALKP GLSAYADDVE KSAQGIRELL DVAKQDIPFD FWKATPLVLK |
| 181 | ATAGLRLLPG EKAQKLLQKV KEVFKASPFL VGDDCVSIMN GTDEGVSAWI TINFLTGSLK |
| 241 | TPGGSSVGML DLGGGSTQIA FLPRVEGTLQ ASPPGYLTAL RMFNRTYKLY SYSYLGLGLM |
| 301 | SARLAILGGV EGQPAKDGKE LVSPCLSPSF KGEWEHAEVT YRVSGQKAAA SLHELCAARV |
| 361 | SEVLQNRVHR TEEVKHVDFY AFSYYYDLAA GVGLIDAEKG GSLVVGDFEI AAKYVCRTLE |
| 421 | TQPQSSPFSC MDLTYVSLLL QEFGFPRSKV LKLTRKIDNV ETSWALGAIF HYIDSLNRQK |
| 481 | SPAS. |
| Human CD39-L3, also known as NTPDase3 or ENTPD3: | |
| (SEQ ID NO: 42) | |
| 1 | MFTVLTRQPC EQAGLKALYR TPTIIALVVL LVSIVVLVSI TVIQIHKQEV LPPGLKYGIV |
| 61 | LDAGSSRTTV YVYQWPAEKE NNTGVVSQTF KCSVKGSGIS SYGNNPQDVP RAFEECMQKV |
| 121 | KGQVPSHLHG STPIHLGATA GMRLLRLQNE TAANEVLESI QSYFKSQPFD FRGAQIISGQ |
| 181 | EEGVYGWITA NYLMGNFLEK NLWHMWVHPH GVETTGALDL GGASTQISFV AGEKMDLNTS |
| 241 | DIMQVSLYGY VYTLYTHSFQ CYGRNEAEKK FLAMLLQNSP TKNHLTNPCY PRDYSISFTM |
| 301 | GHVFDSLCTV DQRPESYNPN DVITFEGTGD PSLCKEKVAS IFDFKACHDQ ETCSFDGVYQ |
| 361 | PKIKGPFVAF AGFYYTASAL NLSGSFSLDT FNSSTWNFCS QNWSQLPLLL PKFDEVYARS |
| 421 | YCFSANYIYH LFVNGYKFTE ETWPQIHFEK EVGNSSIAWS LGYMLSLTNQ IPAESPLIRL |
| 481 | PIEPPVFVGT LAFFTAAALL CLAFLAYLCS ATRRKRHSEH AFDHAVDSD. |
| Human CD39-L4, also known as NTPDase5 or ENTPD5: | |
| (SEQ ID NO: 43) | |
| 1 | MATSWGTVFF MLVVSCVCSA VSHRNQQTWF EGIFLSSMCP INVSASTLYG IMFDAGSTGT |
| 61 | RIHVYTFVQK MPGQLPILEG EVFDSVKPGL SAFVDQPKQG AETVQGLLEV AKDSIPRSHW |
| 121 | KKTPVVLKAT AGLRLLPEHK AKALLFEVKE IFRKSPFLVP KGSVSIMDGS DEGILAWVTV |
| 181 | NFLTGQLHGH RQETVGTLDL GGASTQITFL PQFEKTLEQT PRGYLTSFEM FNSTYKLYTH |
| 241 | SYLGFGLKAA RLATLGALET EGTDGHTFRS ACLPRWLEAE WIFGGVKYQY GGNQEGEVGF |
| 301 | EPCYAEVLRV VRGKLHQPEE VQRGSFYAFS YYYDRAVDTD MIDYEKGGIL KVEDFERKAR |
| 361 | EVCDNLENFT SGSPFLCMDL SYITALLKDG FGFADSTVLQ LTKKVNNIET GWALGATFHL |
| 421 | LQSLGISH. |

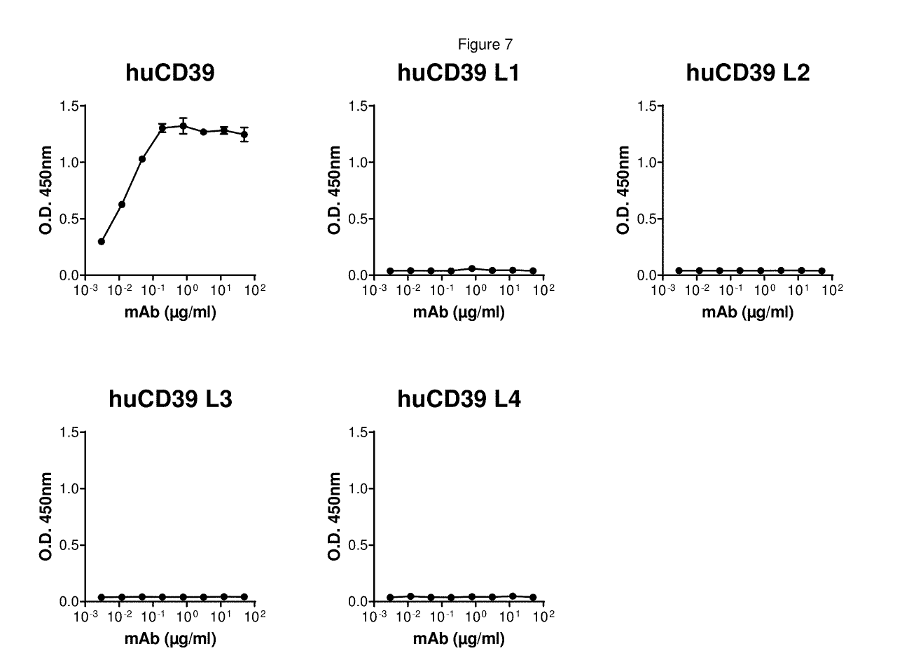

I-394 bound to the CD39 but not to any of the isoforms CD39-L1, -L2, -L3 or -L4. Isotype control antibodies (IC) did not bind to any CD39 or CD39-L molecule. Results are shown in FIG. 7.

### Example 11: Activation of Dendritic Cells

While ATP has pro-inflammatory activity, CD39-mediated catabolism of ATP is believed to be able to impair dendritic cell (DC) activation, in turn altering a broader adaptive immune response against tumor antigen. In order to evaluate whether CD39 blockade using anti-CD39 antibodies could overcome CD39-mediated alteration of dendritic cell (DC) activation in the presence of ATP, we incubated monocyte-derived DC (moDC) with anti-CD39 antibodies in the presence of ATP.

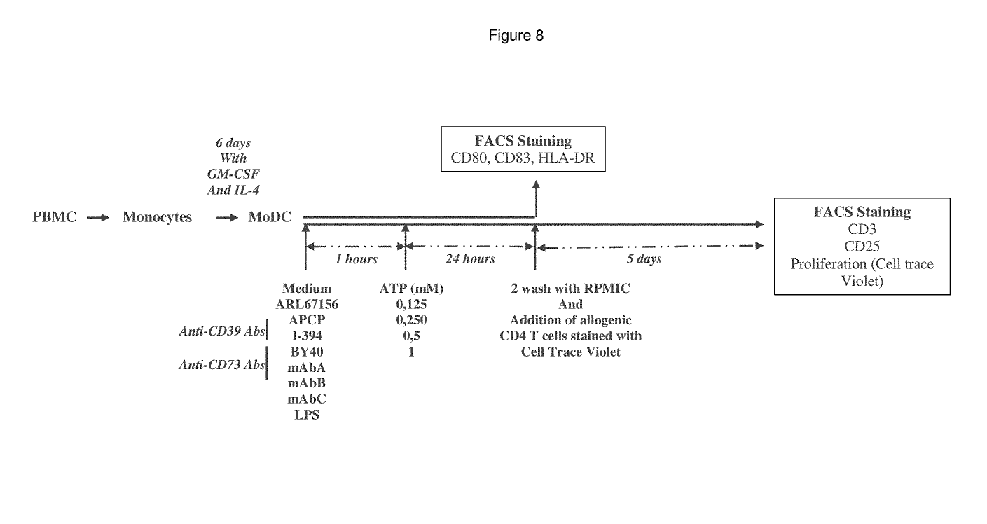

Briefly, human monocytes were purified from human healthy blood and differentiated into MoDC in presence of GM-CSF and IL-4 during 6 days. Then MoDC were activated in presence of ATP (Sigma, 0.25-1 mM) during 24 hours and DC activation were assessed by analyzing CD80, CD83 and HLA-DR expression by flow cytometry. In some cases, MoDC were preincubated during 1 hours in presence of CD39 inhibitor: ARL6716 (Tocris, 250 μM), CD73 inhibitor: APCP (Tocris 50 μM), anti-CD39 blocking antibody I-394 or BY40 (for BY40 see WO2009/095478), or anti-CD73 blocking antibodies. LPS (Invivogen, 10 ng/ml) was used as positive control. To assess resulting effect of ATP-mediated DC activation on CD4 T cells activation, ATP-activated DC were washed and then incubated with allogenic CD4 T cells (ratio 1 MoDC/4 T cells) for a mixed lymphocytes reaction (MLR) during 5 days. T cells activation and proliferation were analyzed through CD25 expression and Cell Trace Violet dilution by flow cytometry (FIG. 8).

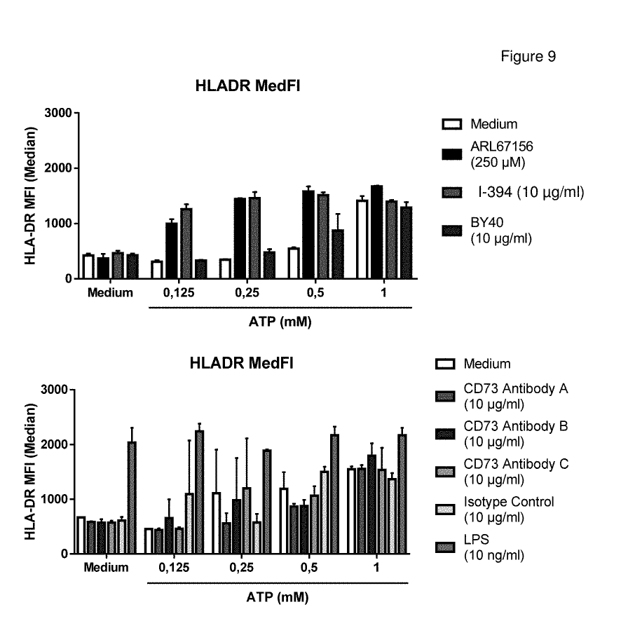

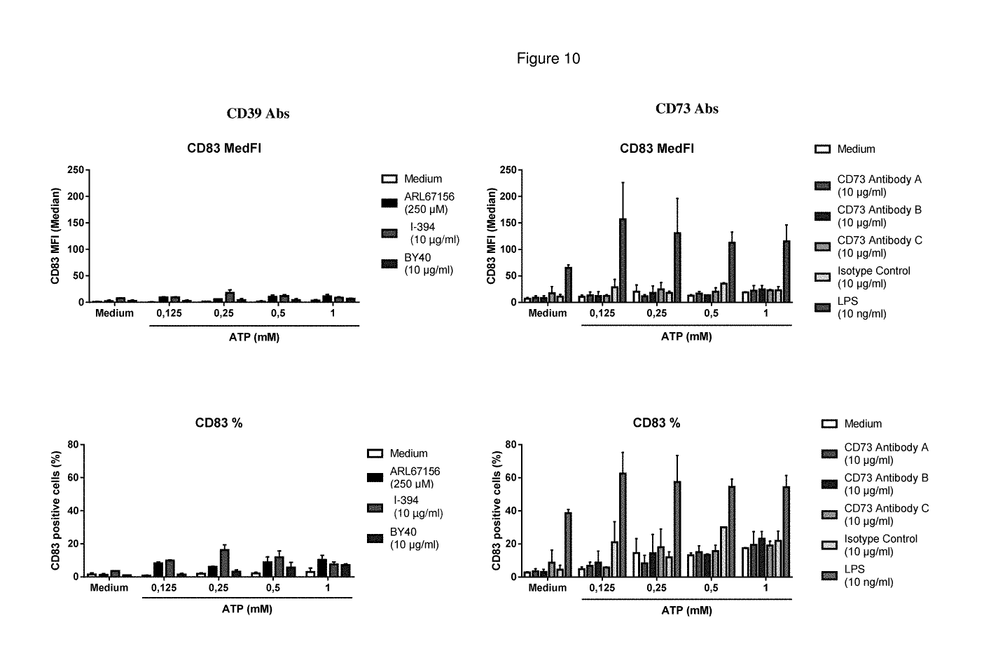

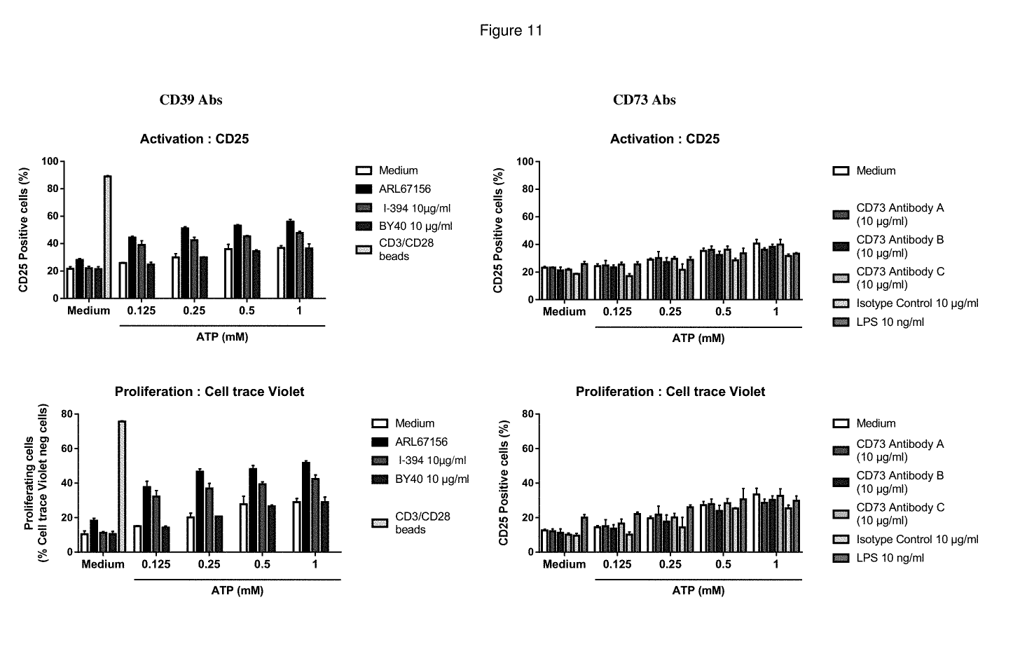

Results are shown in FIGS. 9, 10 and 11. In the presence of negative control (medium), moDC activation was observed in the presence of 1 mM ATP, however ATP at 0.125 mM, 0.25 mM or 0.5 mM did not permit moDC activation. Addition of chemical inhibitors of CD39 which are believed to fully block CD39 enzymatic activity by binding to the active site lead to moDC activation at each of 0.125 mM, 0.25 mM or 0.5 mM. However, anti-CD39 antibodies such as BY40 or anti-CD73 antibodies were not able to favor ATP-induced activation of dendritic cell (DC), suggesting that antibodies are not able to block enzymatic activity sufficiently to avoid ATP catabolism. Surprisingly, the anti-CD39 blocking antibody I-394 (shown in Figures at concentration 10 μg/ml) which substantially fully blocks the ATPase activity of CD39 and can therefore permit accumulation of ATP, permitted moDC activation as assessed by HLA-DR or CD83 expression at each of 0.125 mM, 0.25 mM or 0.5 mM (FIGS. 9 and 10). Interestingly, the MoDC activated in presence of ATP were able to induce better T cells activation and proliferation in a MLR assay. Moreover, the enhancement of ATP-mediated MoDC activation by anti-CD39 blocking antibody I-394 resulted in higher T cells proliferation and activation (FIG. 11).

Assessment of the ability to CD39 inhibitors to activate DC in the presence of ATP provides a method to identify and evaluate anti-CD39 antibodies that are able to achieve a high degree of inhibition of CD39. Furthermore, the possibility of using anti-CD39 antibodies to relieve the immunosuppressive effect exerted by CD39 upon DC can provide for enhancement of the adaptive immune response toward antigens, notably on tumors cells. Furthermore, such anti-CD39 antibodies may be of particular interest when used to enhance the immunogenic effect of chemotherapeutic agents. Numerous chemotherapeutic agents that cause necrosis of tumor cells are able to induce ATP; combined use with anti-CD39 antibodies can be particularly useful to enhance the anti-tumor response in these settings.

### Example 12: In Vivo Combination Treatment with Anti-CD39 Antibodies of Agents that Induce ATP Release

1×10⁶ MCA205 mouse tumor cells (sarcoma) were subcutaneously engrafted in the right flank of mice genetically modified to express human CD39 (CD39KI mice). Mice (n=9 to 13) were either treated with controls, oxaliplatin (10 mg/kg, intraperitoneally, on days 5 and 12 or 14), murine anti-human CD39 antibody I-394 (20 mg/kg for the first injection and then 10 mg/kg, intra-venously, twice a week for 3 or 4 weeks from day 4) or combination of both. Tumors were measured twice a week with a caliper (L: length and w: width) and tumor volume was calculated with the formula (L×w2)/2. Mice were sacrificed when tumor volume was above 1800 mm³ or when tumors were highly necrosed. The human CD39 KI mice were engrafted with MCA205 tumor cells at day 0. The murine I-394 used was engineered with an aglycosylated mouse IgG1 isotype (substitution at Kabat heavy chain residue N297), in order to prevent mouse FcR and complement binding, such that the only effect observed is linked to the blocking property of the antibody, and not to ADCC or CDC lysis of CD39+ immune suppressor cells or CD39+ endothelial cells.

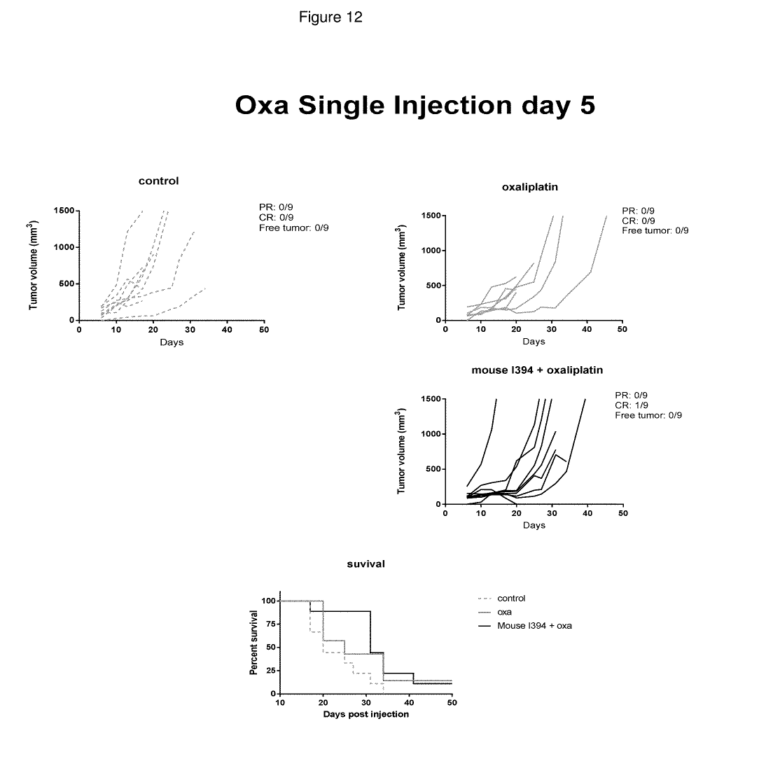

In a first experimental series, mice were treated at day 5 post tumor cell engraftment with either control (1 group) PBS, or oxaliplatin chemotherapy (2 groups). In parallel, one group of mice treated with oxaliplatin was injected twice a week with anti-CD39 antibody, with the anti-CD39 antibody treatment starting just one day before oxaliplatin treatment (at day 4). This ensured that oxaliplatin induced ATP release in a tumor environment where CD39 was already and fully inhibited, and thus providing optimal prevention of ATP degradation by intratumoral CD39. In this experiment, a delay of tumor growth and mice survival could be observed in the combination of oxaliplatin and I-394 antibody group, however the delay was deemed relatively modest, even though one Complete Response (CR) was obtained in this group, whereas no CR was observed in the control or oxaliplatin single agent group. Survival median of control was 20 days, oxaliplatin was 25 days and I-394 antibody combined with oxaliplatin was 31 days. Results are shown in FIG. 12.

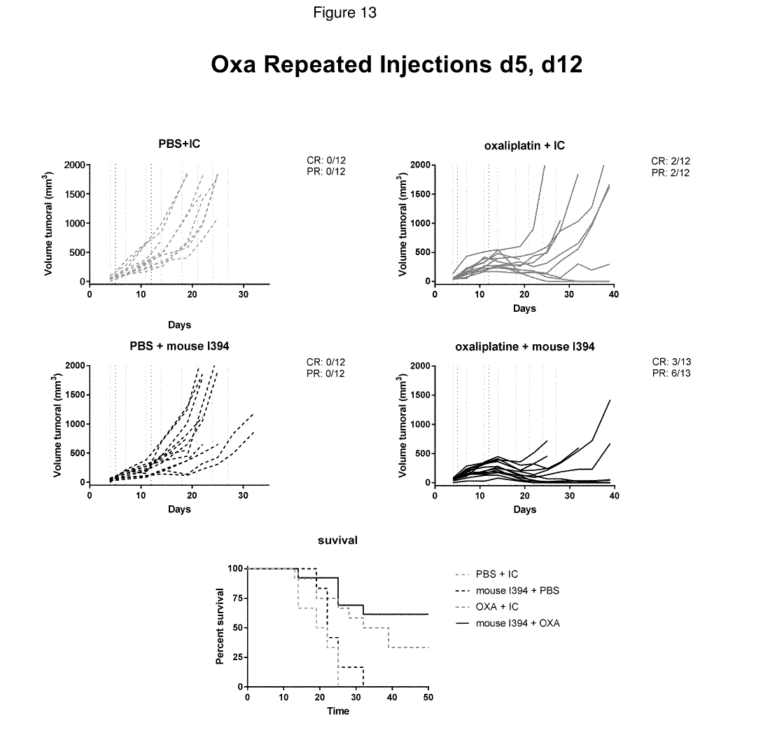

In a second experimental series (one representative experiment out of 2 is shown), the oxaliplatin injection was repeated one week after the first oxaliplatin injection, again just one day after the treatment with I-394 antibody, to provide optimal inhibition of ATP degradation. I-394 Ab administered alone had only marginal effect on tumor growth and on mice survival. Oxaliplatin as single agent with repeated (twice) injections did induce some regression of tumor volume, and increase in mice survival. However, the combination of repeated injections of oxaliplatin combined with antibody I-394 administered before oxaliplatin induced tumor volume regression in all mice, with 3 CR compared with 2 with Oxaliplatine alone, and 6 partial responses (PR) versus 3 PR with Oxaliplatine alone. The combination also improved survival of mice, 40 days post tumor engraftment, with 4/13 tumor free mice versus 2/12 in the oxaliplatin alone group. Results are shown in FIG. 13.

The second repeat injection of oxaliplatin in this setting is believed to allow ATP accumulation and adenosine (Ado) suppression in the presence of blocking anti-CD39 antibody to take place during the classical two week period required to mount an efficient anti-tumor immune response. Consequently, in humans an improved treatment regimen can involve repeating the chemotherapy administration at least twice, in order to see the strongest combination effect with anti-CD39 blocking antibody. Moreover, the anti-CD39 Ab can ideally be administered at least 1-48 h before chemotherapeutic agent inducing ATP release, in order to ensure full inhibition of intratumoral CD39, and full inhibition of ATP degradation into adenosine, at the moment when the chemotherapeutic agent induces ATP release.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of treating a tumor in a human individual, the treatment comprising administering to the individual an effective amount of each of: (a) an antibody that is capable of binding and inhibiting the ATPase activity of a soluble extracellular domain human CD39 in the presence of exogenously added ATP, and (b) an agent or treatment that induces the extracellular release of ATP from tumor cells.

2. The method of claim 1, wherein the antibody is capable of binding and inhibiting the ATPase activity of a soluble extracellular domain human CD39 protein, optionally wherein the antibody is capable of causing a decrease in the ATPase activity of the human extracellular domain CD39 protein in solution by more than 50%, optionally more than 60%, 70%, 75% or 80%.

3. The method of claim 2, wherein the antibody inhibits the ATPase activity of the soluble extracellular domain human CD39 protein in the presence of exogenously added ATP, wherein ATP is provided at a concentration of 20 μM.

4. A method of sensitizing an individual to treatment with an agent or treatment that induces the extracellular release of ATP from tumor cells, comprising administering to the individual an antibody that is capable of binding and inhibiting the ATPase activity of a soluble CD39 in the presence of exogenously added ATP.

5. The method of claim 1, wherein the agent or treatment that induces the extracellular release of ATP from tumor cells is an agent that is induces the death of tumor cells.

6. The method of claim 1, wherein the individual has a cancer that has relapsed or progressed following a prior course of treatment with an agent capable of inducing the extracellular release of ATP from tumor cells.

7. The method of claim 1, wherein the individual has a cancer that has relapsed or progressed following a prior course of treatment with a platinum agent, a taxane or a PARP inhibitor.

8. The method of claim 1, wherein the method or treatment further comprises administering to the individual (a) an agent, optionally an antibody, that neutralizes the inhibitory activity of human PD-1 and/or (b) an agent, optionally an antibody or small molecule agent, that neutralizes the inhibitory activity of a human CD73 protein.

9. The method of claim 1, wherein the individual has a poor response or prognostic for response to treatment with an agent that induces the extracellular release of ATP from tumor cells, in the absence of combined treatment with an antibody that is capable of binding and neutralizing the ATPase activity of human CD39.

10. The method of claim 1, wherein an antibody that is capable of binding and inhibiting the ATPase activity of CD39 in the presence of exogenously added ATP antibody is an antibody that is capable of causing an increase in expression of a cell surface marker of activation in monocyte-derived dendritic cells, when such moDC are incubated in vitro with the antibody and ATP, optionally wherein exogenously added ATP is provided at 0.125 mM, 0.25 mM or 0.5 mM.

11. The method of claim 1, wherein the antibody is capable of binding and neutralizing the ATPase activity of human CD39 at the surface of a cell.

12. The method of claim 1, wherein the antibody is capable of increasing the activation of dendritic cells in the presence of ATP.

13. The method of claim 1, wherein the antibody is capable of causing an increase in expression of a cell surface marker of activation in monocyte-derived dendritic cells, when such moDC are incubated in vitro with the antibody and ATP.

14. The method of claim 10, wherein an increase in expression of a cell surface marker of activation is assessed by incubating moDC in presence of ATP for 24 hours and analyzing cell surface expression of CD80, CD83 and/or HLA-DR on moDC by flow cytometry.

15. The method of claim 10, wherein the increase in expression of a cell surface marker is at least 40%.

16. The method of claim 1, wherein the antibody is capable of increasing proliferation of T cells, when T cells co-cultured in vitro with CD39-expressing DC cells, in the presence of ATP.

17. The method of claim 1, wherein the agent or treatment that induces the extracellular release of ATP from tumor cells is selected from the group consisting of a PARP inhibitor, taxane, a platinum agent, and radiotherapy.

18. The method of claim 1, wherein the antibody that neutralizes the ATPase activity of human CD39 and agent that induces the extracellular release of ATP from tumor cells are formulated for separate administration and are administered concurrently or sequentially.

19. The method of claim 1, wherein the agent that induces the extracellular release of ATP from tumor cells is administered 1 to 48 hours after the administration of the antibody that is capable of binding and inhibiting the ATPase activity of CD39.

20. The method of claim 1, wherein the individual has a hematological tumor or a solid tumor selected from the group consisting of an ovarian cancer, a gastric cancer, a lung cancer, a colon cancer and an esophageal cancer.

21. The method of claim 20, wherein the individual has a platinum-sensitive cancer.

22. The method of claim 20, wherein the individual has a platinum-resistant cancer.

23. The method of claim 20, wherein the individual has a taxane-sensitive cancer.

24. The method of claim 20, wherein the individual has a taxane-resistant cancer.

25. The method of claim 20, wherein the individual has a PARP inhibitor-sensitive cancer.

26. The method of claim 20, wherein the individual has a PARP inhibitor-resistant cancer.

27. A pharmaceutical composition comprising an antibody that is capable of binding and inhibiting the ATPase activity of a soluble extracellular domain human CD39 protein, and an agent that induces the extracellular release of ATP from tumor cells.

28. A kit comprising: (a) a dose of an antibody that is capable of binding and inhibiting the ATPase activity of a soluble extracellular domain human CD39 protein, and (b) a dose of an agent that induces the extracellular release of ATP from tumor cells.

29. A kit comprising: (a) multiple packages of single-dose pharmaceutical compositions containing an effective amount of an antibody that is capable of binding and inhibiting the ATPase activity of a soluble extracellular domain human CD39 protein, and (b) multiple packages of single-dose pharmaceutical compositions containing an agent that induces the extracellular release of ATP from tumor cells.

30. A method of enhancing an adaptive anti-tumor response in an individual, optionally further for enhancing dendritic cell activation and/or dendritic cell mediated T cell proliferation, in an individual, the method comprising administering to said individual an effective amount of an antibody that is capable of neutralizing the ATPase activity of CD39, and an agent that induces the extracellular release of ATP from tumor cells.

31. A method of treating cancer in an individual whose cancer has relapsed or progressed following treatment with an agent capable of inducing the extracellular release of ATP from tumor cells, comprising administering to the individual an antibody that is capable of binding and inhibiting the ATPase activity of a soluble extracellular domain human CD39 protein.

32. The method of claim 31, wherein the treatment further comprises administering to the individual an agent that induces the extracellular release of ATP from tumor cells.

33. The method of claim 32, wherein the agent capable of inducing the extracellular release of ATP from tumor cells is a platinum agent, a taxane or a PARP inhibitor.

34. The method of claim 1, wherein the antibody has reduced binding to:
(a) a mutant CD39 polypeptide comprising the mutations Q96A, N99A, E143A and R147E (with reference to SEQ ID NO: 1);
(b) a mutant CD39 polypeptide comprising a mutation at 1, 2, 3 or 4 residues selected from the group consisting of D150, E153 and R154 (with reference to SEQ ID NO:
1), relative to binding between the antibody and a wild-type CD39 polypeptide comprising the amino acid sequence of SEQ ID NO: 1;
(c) a mutant CD39 polypeptide comprising a mutation at 1, 2, 3 or 4 residues selected from the group consisting of N99, E153 and R154 (with reference to SEQ ID NO:
1), relative to binding between the antibody and a wild-type CD39 polypeptide comprising the amino acid sequence of SEQ ID NO: 1;
(d) a mutant CD39 polypeptide comprising the mutations R138A, M139A and E142K (with reference to SEQ ID NO: 1);
(e) a mutant CD39 polypeptide comprising the mutations K87A, E100A and D107A (with reference to SEQ ID NO: 1); and/or
(f) a mutant CD39 polypeptide comprising the mutations N371K, L372K, E375A, K376G and V377S, and an insertion of a valine between residues 376 and 377 (with reference to SEQ ID NO: 1);
in each case, relative to binding between the antibody and a wild-type CD39 polypeptide comprising the amino acid sequence of SEQ ID NO: 1.

35. The method of claim 1, wherein the anti-CD39 antibody comprises a heavy chain variable region that is a function-conservative variant of the heavy chain variable region of antibody I-394, I-395, I-396, I-397, I-398 or I-399, and a light chain variable region that is a function-conservative variant of the light chain variable region of the respective I-394, I-395, I-396, I-397, I-398 or I-399 antibody.

36. The method of claim 1, wherein the antibody that neutralizes the ATPase activity of CD39 comprises the heavy chain CDR1, CDR2 and CDR3 domains of the heavy chain variable domain having the sequence set forth in SEQ ID NOS: 6, and the light chain CDR1, CDR2 and CDR3 domains of the light chain variable domain having the sequence set forth in SEQ ID NO: 7.

37. The method of claim 1, wherein the antibody that neutralizes the ATPase activity of CD39 comprises the heavy chain CDR1, CDR2 and CDR3 domains of the heavy chain variable domain having the sequence set forth in SEQ ID NOS: 81, and the light chain CDR1, CDR2 and CDR3 domains of the light chain variable domain having the sequence set forth in SEQ ID NO: 82.

38. The method of claim 1, wherein the anti-CD39 antibody comprises a HCDR1 comprising an amino acid sequence DYNMH (SEQ ID NO: 5); a HCDR2 comprising an amino acid sequence YIVPLNGGSTFNQKFKG (SEQ ID NO: 6); a HCDR3 comprising an amino acid sequence GGTRFAY (SEQ ID NO: 7); a LCDR1 comprising an amino acid sequence RASESVDNFGVSFMY (SEQ ID NO: 8); a LCDR2 region comprising an amino acid sequence GASNQGS (SEQ ID NO: 9); and a LCDR3 region comprising an amino acid sequence QQTKEVPYT (SEQ ID NO: 10).

39. The method of claim 1, wherein the anti-CD39 antibody comprises a HCDR1 comprising an amino acid sequence SYEMH (SEQ ID NO: 83); a HCDR2 comprising an amino acid sequence RINPSVGSTWYAQKFQG (SEQ ID NO: 74); a HCDR3 comprising an amino acid sequence GKREGGTEYLRK (SEQ ID NO: 85); a LCDR1 comprising an amino acid sequence RASQSVASSYLA (SEQ ID NO: 86); a LCDR2 region comprising an amino acid sequence GASNRHT (SEQ ID NO: 87); and a LCDR3 region comprising an amino acid sequence QQYHNAIT (SEQ ID NO: 88).

40. The method of claim 1, wherein the antibody is administered at least 1 hour prior to the administration of the agent or treatment that induces the extracellular release of ATP.

41. The method of claim 1, wherein the agent or treatment that induces the extracellular release of ATP is administered at least twice within a period of two weeks following administration of the antibody.

42. In a method of treating a tumor in a human individual by administering an agent or treatment that induces the extracellular release of ATP, the improvement comprising further administering to the individual an effective amount of an antibody that is capable of binding and inhibiting the ATPase activity of a soluble extracellular domain human CD39 in the presence of exogenously added ATP.

43. The method of claim 42, wherein the antibody is administered at least 1 hour prior to the administration of the agent or treatment that induces the extracellular release of ATP.

44. The method of claim 42, wherein the agent or treatment that induces the extracellular release of ATP is administered at least twice within a period of two weeks following administration of the antibody.

45. A method of treating a tumor in a human individual, the treatment comprising administering to the individual an effective amount of each of: (a) a means for inhibiting the ATPase activity of a soluble extracellular domain human CD39, and (b) a means for inducing the extracellular release of ATP from tumor cells.

46. In a method of treating a tumor in a human individual by administering an agent or treatment that induces the extracellular release of ATP, the improvement comprising further administering to the individual an effective amount of means for inhibiting the ATPase activity of a soluble extracellular domain human CD39.


SEQUENCE LISTING (90 sequences)

SEQ ID NO: 1 - protein, 510 aa, HOMO SAPIENS
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 2 - protein, 441 aa, HOMO SAPIENS
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYV

SEQ ID NO: 3 - protein, 116 aa, MUS MUSCULUS
EVQLQQSGPELVKPGASVKMSCKASGYTFTDYNMHWVKQSHGRTLEWIGYIVPLNGGSTFNQKFKGRATLTVNTSSRTAYMELRSLTSEDSAAYYCARGGTRFAYWGQGTLVTVSA

SEQ ID NO: 4 - protein, 111 aa, MUS MUSCULUS
DIVLTQSPASLAVSLGQRATISCRASESVDNFGVSFMYWFQQKPGQPPNLLIYGASNQGSGVPARFRGSGSGTDFSLNIHPMEADDTAMYFCQQTKEVPYTFGGGTKLEIK

SEQ ID NO: 5 - protein, 5 aa, MUS MUSCULUS
DYNMH

SEQ ID NO: 6 - protein, 17 aa, Mus musculus
YIVPLNGGSTFNQKFKG

SEQ ID NO: 7 - protein, 7 aa, Mus musculus
GGTRFAY

SEQ ID NO: 8 - protein, 15 aa, Mus musculus
RASESVDNFGVSFMY

SEQ ID NO: 9 - protein, 7 aa, Mus musculus
GASNQGS

SEQ ID NO: 10 - protein, 9 aa, Mus musculus
QQTKEVPYT

SEQ ID NO: 11 - protein, 116 aa, Mus musculus
EVQLQQSGPELVKPGASVRMSCKASGYTFTDYNMHWVKKNHGKGLEWIGYINPNNGGTTYNQKFKGKATLTVNTSSKTAYMELRSLTSEDSAVYYCTRGGTRFASWGQGTLVTVSA

SEQ ID NO: 12 - protein, 111 aa, Mus musculus
NIVLTQSPASLAVSLGQRATISCRASESVDNYGISFMYWFQQKPGQPPKLLIYAASTQGSGVPARFSGSGSGTDFSLNIHPMEEDDTAMYFCQQSKEVPFTFGSGTKLEIK

SEQ ID NO: 13 - protein, 5 aa, Mus musculus
DYNMH

SEQ ID NO: 14 - protein, 17 aa, Mus musculus
YINPNNGGTTYNQKFKG

SEQ ID NO: 15 - protein, 7 aa, Mus musculus
GGTRFAS

SEQ ID NO: 16 - protein, 15 aa, Mus musculus
RASESVDNYGISFMY

SEQ ID NO: 17 - protein, 7 aa, Mus musculus
AASTQGS

SEQ ID NO: 18 - protein, 9 aa, Mus musculus
QQSKEVPFT

SEQ ID NO: 19 - protein, 122 aa, Mus musculus
EVQLQQSGAELVKPGASVKLSCIVSGFNIKDTYINWVKQRPEQGLEWIGRIDPANGNTKYDPKFQGKATMTSDTSSNTAYLHLSSLTSDDSAVYYCARWGYDDEEADYFDSWGQGTTLTVSS

SEQ ID NO: 20 - protein, 111 aa, Mus musculus
DIVLTQSPASLAVSLGQRATISCRASESVDNYGISFMNWFQQKPGQPPKLLIYAASNQGSGVPARFSGSGSGTDFSLNILPMEEVDAAMYFCHQSKEVPWTFGGGTKLEIK

SEQ ID NO: 21 - protein, 5 aa, Mus musculus
DTYIN

SEQ ID NO: 22 - protein, 17 aa, Mus musculus
RIDPANGNTKYDPKFQG

SEQ ID NO: 23 - protein, 13 aa, Mus musculus
WGYDDEEADYFDS

SEQ ID NO: 24 - protein, 15 aa, Mus musculus
RASESVDNYGISFMN

SEQ ID NO: 25 - protein, 7 aa, Mus musculus
AASNQGS

SEQ ID NO: 26 - protein, 9 aa, Mus musculus
HQSKEVPWT

SEQ ID NO: 27 - protein, 118 aa, Mus musculus
PVQLQQPGAEVVMPGASVKLSCKASGYTFTSFWMNWMRQRPGQGLEWIGEIDPSDFYTNSNQRFKGKATLTVDKSSSTAYMQLSSLTSEDSAVYFCARGDFGWYFDVWGTGTSVTVSS

SEQ ID NO: 28 - protein, 108 aa, Mus musculus
EIVLTQSPTTMTSSPGEKITFTCSASSSINSNYLHWYQQKPGFSPKLLIYRTSNLASGVPTRFSGSGSGTSYSLTIGTMEAEDVATYYCQQGSSLPRTFGGGTKLEIK

SEQ ID NO: 29 - protein, 5 aa, Mus musculus
SFWMN

SEQ ID NO: 30 - protein, 17 aa, Mus musculus
EIDPSDFYTNSNQRFKG

SEQ ID NO: 31 - protein, 9 aa, Mus musculus
GDFGWYFDV

SEQ ID NO: 32 - protein, 12 aa, Mus musculus
SASSSINSNYLH

SEQ ID NO: 33 - protein, 7 aa, Mus musculus
RTSNLAS

SEQ ID NO: 34 - protein, 9 aa, Mus musculus
QQGSSLPRT

SEQ ID NO: 35 - DNA, 34 bp, HOMO SAPIENS
acaggcgtgcattcgcagaacaaagcattgccag

SEQ ID NO: 36 - DNA, 64 bp, HOMO SAPIENS
ccgccccgactctagatcacttgtcatcgtcatctttgtaatcgacataggtggagtgggagag

SEQ ID NO: 37 - protein, 446 aa, Artificial
  Chimeric homo sapiens mus musculus
EVQLQQSGPELVKPGASVKMSCKASGYTFTDYNMHWVKQSHGRTLEWIGYIVPLNGGSTFNQKFKGRATLTVNTSSRTAYMELRSLTSEDSAAYYCARGGTRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 218 aa, Artificial
  Chimeric homo sapiens mus musculus
DIVLTQSPASLAVSLGQRATISCRASESVDNFGVSFMYWFQQKPGQPPNLLIYGASNQGSGVPARFRGSGSGTDFSLNIHPMEADDTAMYFCQQTKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 449 aa, HOMO SAPIENS
TQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVDYKDDDDK

SEQ ID NO: 40 - protein, 472 aa, HOMO SAPIENS
MAGKVRSLLPPLLLAAAGLAGLLLLCVPTRDVREPPALKYGIVLDAGSSHTSMFIYKWPADKENDTGIVGQHSSCDVPGGGISSYADNPSGASQSLVGCLEQALQDVPKERHAGTPLYLGATAGMRLLNLTNPEASTSVLMAVTHTLTQYPFDFRGARILSGQEEGVFGWVTANYLLENFIKYGWVGRWFRPRKGTLGAMDLGGASTQITFETTSPAEDRASEVQLHLYGQHYRVYTHSFLCYGRDQVLQRLLASALQTHGFHPCWPRGFSTQVLLGDVYQSPCTMAQRPQNFNSSARVSLSGSSDPHLCRDLVSGLFSFSSCPFSRCSFNGVFQPPVAGNFVAFSAFFYTVDFLRTSMGLPVATLQQLEAAAVNVCNQTWAQQLLSRGYGFDERAFGGVIFQKKAADTAVGWALGYMLNLTNLIPADPPGLRKGTDFSSWVVLLLLFASALLAALVLLLRQVHSAKLPSTI

SEQ ID NO: 41 - protein, 484 aa, HOMO SAPIENS
MKKGIRYETSRKTSYIFQQPQHGPWQTRMRKISNHGSLRVAKVAYPLGLCVGVFIYVAYIKWHRATATQAFFSITRAAPGARWGQQAHSPLGTAADGHEVFYGIMFDAGSTGTRVHVFQFTRPPRETPTLTHETFKALKPGLSAYADDVEKSAQGIRELLDVAKQDIPFDFWKATPLVLKATAGLRLLPGEKAQKLLQKVKEVFKASPFLVGDDCVSIMNGTDEGVSAWITINFLTGSLKTPGGSSVGMLDLGGGSTQIAFLPRVEGTLQASPPGYLTALRMFNRTYKLYSYSYLGLGLMSARLAILGGVEGQPAKDGKELVSPCLSPSFKGEWEHAEVTYRVSGQKAAASLHELCAARVSEVLQNRVHRTEEVKHVDFYAFSYYYDLAAGVGLIDAEKGGSLVVGDFEIAAKYVCRTLETQPQSSPFSCMDLTYVSLLLQEFGFPRSKVLKLTRKIDNVETSWALGAIFHYIDSLNRQKSPAS

SEQ ID NO: 42 - protein, 529 aa, HOMO SAPIENS
MFTVLTRQPCEQAGLKALYRTPTIIALVVLLVSIVVLVSITVIQIHKQEVLPPGLKYGIVLDAGSSRTTVYVYQWPAEKENNTGVVSQTFKCSVKGSGISSYGNNPQDVPRAFEECMQKVKGQVPSHLHGSTPIHLGATAGMRLLRLQNETAANEVLESIQSYFKSQPFDFRGAQIISGQEEGVYGWITANYLMGNFLEKNLWHMWVHPHGVETTGALDLGGASTQISFVAGEKMDLNTSDIMQVSLYGYVYTLYTHSFQCYGRNEAEKKFLAMLLQNSPTKNHLTNPCYPRDYSISFTMGHVFDSLCTVDQRPESYNPNDVITFEGTGDPSLCKEKVASIFDFKACHDQETCSFDGVYQPKIKGPFVAFAGFYYTASALNLSGSFSLDTFNSSTWNFCSQNWSQLPLLLPKFDEVYARSYCFSANYIYHLFVNGYKFTEETWPQIHFEKEVGNSSIAWSLGYMLSLTNQIPAESPLIRLPIEPPVFVGTLAFFTAAALLCLAFLAYLCSATRRKRHSEHAFDHAVDSD

SEQ ID NO: 43 - protein, 428 aa, HOMO SAPIENS
MATSWGTVFFMLVVSCVCSAVSHRNQQTWFEGIFLSSMCPINVSASTLYGIMFDAGSTGTRIHVYTFVQKMPGQLPILEGEVFDSVKPGLSAFVDQPKQGAETVQGLLEVAKDSIPRSHWKKTPVVLKATAGLRLLPEHKAKALLFEVKEIFRKSPFLVPKGSVSIMDGSDEGILAWVTVNFLTGQLHGHRQETVGTLDLGGASTQITFLPQFEKTLEQTPRGYLTSFEMFNSTYKLYTHSYLGFGLKAARLATLGALETEGTDGHTFRSACLPRWLEAEWIFGGVKYQYGGNQEGEVGFEPCYAEVLRVVRGKLHQPEEVQRGSFYAFSYYYDRAVDTDMIDYEKGGILKVEDFERKAREVCDNLENFTSGSPFLCMDLSYITALLKDGFGFADSTVLQLTKKVNNIETGWALGATFHLLQSLGISH

SEQ ID NO: 44 - protein, 330 aa, HOMO SAPIENS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 330 aa, HOMO SAPIENS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 330 aa, HOMO SAPIENS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 330 aa, HOMO SAPIENS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 574 aa, homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 49 - protein, 120 aa, Artificial
  chimeric
QVQLVQSGAEVKKPGSSVKVSCKASGGTFPSNAISWVRQAPGQGLEWMGGIGFGTANYAQKFQGRVTITADESTSTAYMELSSLRSEDTAVYYCARGGAKYARTYGMDVWGQGTTVTVSS

SEQ ID NO: 50 - protein, 113 aa, Artificial
  Chimeric
DIVMTQSPDSLAVSLGERATINCKSSKSVLYSNNNKNYLAWYQQKPGQPPKLLIYWASTRQSGVPDRFSGSGSGTDFTLTISSLQAEDVAVYYCQQYLLYPLTFGGGTKVEIK

SEQ ID NO: 51 - protein, 5 aa, Artificial
  Synthetic CDR
SNAIS

SEQ ID NO: 52 - protein, 15 aa, Artificial
  Synthetic CDR
GIGFGTANYAQKFQG

SEQ ID NO: 53 - protein, 13 aa, Artificial
  Synthetic CDR
GGAKYARTYGMDV

SEQ ID NO: 54 - protein, 17 aa, Artificial
  Synthetic CDR
KSSKSVLYSNNNKNYLA

SEQ ID NO: 55 - protein, 7 aa, Artificial
  Synthetic CDR
WASTRQS

SEQ ID NO: 56 - protein, 9 aa, Artificial
  Synthetic CDR
QQYLLYPLT

SEQ ID NO: 57 - protein, 120 aa, Artificial
  Synthetic CDR
QVQLVQSGAEVKKPGSSVKVSCKASGGTFSSLPISWVRQAPGQGLEWMGGIGFGTANYAQKFQGRVTITADESTSTAYMELSSLRSEDTAVYYCARGGAKYAGRYGMDVWGQGTTVTVSS

SEQ ID NO: 58 - protein, 113 aa, Artificial
  Synthetic VL
GIVMTQSPDSLAVSLGERATINCKSSQSVLFSSNNKNYLAWYQQKPGQPPKLLIYWASTRASGVPDRFSGSGSGTDFTLTISSLQAEDVAVYYCQQYYLYPLTFGGGTKVEIK

SEQ ID NO: 59 - protein, 5 aa, Artificial
  Synthetic CDR
SLPIS

SEQ ID NO: 60 - protein, 13 aa, Artificial
  Synthetic CDR
GIGFGTANYAQKF

SEQ ID NO: 61 - protein, 13 aa, Artificial
  Synthetic CDR
GGAKYAGRYGMDV

SEQ ID NO: 62 - protein, 17 aa, Artificial
  Synthetic CDR
KSSQSVLFSSNNKNYLA

SEQ ID NO: 63 - protein, 7 aa, Artificial
  Synthetic CDR
WASTRAS

SEQ ID NO: 64 - protein, 9 aa, Artificial
  Synthetic CDR
QQYYLYPLT

SEQ ID NO: 65 - protein, 121 aa, Artificial
  Synthetic VH
QVQLVQSGAEVKKPGSSVKVSCKASGGTFSKYGISWVRQAPGQGLEWMGSIIPEFGIANYAQKFQGRVTITADESTSTAYMELSSLRSEDTAVYYCARESGGYRDHRLGVWGQGTMVTVSS

SEQ ID NO: 66 - protein, 107 aa, Artificial
  Synthetic VL
EIVMTQSPATLSVSPGERATLSCRASQSVGSNLAWYQQKPGQAPRLLIYGASTRASGIPARFSGSGSGTEFTLTISSLQSEDFAVYYCQQYLLWPLTFGGGTKVEIK

SEQ ID NO: 67 - protein, 5 aa, Artificial
  Synthetic CDR
KYGIS

SEQ ID NO: 68 - protein, 17 aa, Artificial
  Synthetic CDr
SIIPEFGIANYAQKFQG

SEQ ID NO: 69 - protein, 12 aa, Artificial
  Synthetic CDR
ESGGYRDHRLGV

SEQ ID NO: 70 - protein, 11 aa, Artificial
  Synthetic CDR
RASQSVGSNLA

SEQ ID NO: 71 - protein, 7 aa, Artificial
  Synthetic CDR
GASTRAS

SEQ ID NO: 72 - protein, 9 aa, Artificial
  Synthetic CDR
QQYLLWPLT

SEQ ID NO: 73 - protein, 121 aa, Artificial
  Synthetic VH
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRNWGQGTLVTVSS

SEQ ID NO: 74 - protein, 107 aa, Artificial
  Synthetic VL
EIVLTQSPGTLSLSPGERATLSCRASQSVSSSYLAWYQQKPGQAPRLLIYGASSRATGIPDRFSGSGSGTDFTLTISRLEPEDFAVYYCQQYHSYITFGGGTKVEIK

SEQ ID NO: 75 - protein, 5 aa, Artificial
  Synthetic CDR
SYEMH

SEQ ID NO: 76 - protein, 17 aa, Artificial
  Synthetic CDR
RINPSVGSTWYAQKFQG

SEQ ID NO: 77 - protein, 12 aa, Artificial
  Synthetic CDR
GKREGGTEYLRN

SEQ ID NO: 78 - protein, 12 aa, Artificial
  Synthetic CDR
RASQSVSSSYLA

SEQ ID NO: 79 - protein, 7 aa, Artificial
  synthetic CDR
GASSRAT

SEQ ID NO: 80 - protein, 8 aa, Artificial
  Synthetic CDR
QQYHSYIT

SEQ ID NO: 81 - protein, 121 aa, Artificial
  Synthetic VH
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSS

SEQ ID NO: 82 - protein, 107 aa, Artificial
  Synthetic VL
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIK

SEQ ID NO: 83 - protein, 5 aa, Artificial
  Synthetic CDR
SYEMH

SEQ ID NO: 84 - protein, 17 aa, Artificial
  Synthetic CDR
RINPSVGSTWYAQKFQG

SEQ ID NO: 85 - protein, 12 aa, Artificial
  Synthetic CDR
GKREGGTEYLRK

SEQ ID NO: 86 - protein, 12 aa, Artificial
  Synthetic CDR
RASQSVASSYLA

SEQ ID NO: 87 - protein, 7 aa, Artificial
  Synthetic CDR
GASNRHT

SEQ ID NO: 88 - protein, 8 aa, Artificial
  Synthetic CDR
QQYHNAIT

SEQ ID NO: 89 - protein, 448 aa, Artificial
  Synthetic heavy chain
QVQLVQSGAEVKKPGASVKVSCKASGYTFKSYEMHWVRQAPGQGLEWMGRINPSVGSTWYAQKFQGRVTMTRDTSTSTVYMELSSLRSEDTAVYYCARGKREGGTEYLRKWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 214 aa, Artificial
  Synthetic light chain
EIVLTQSPGTLSLSPGERATLSCRASQSVASSYLAWYQQKPGQAPRLLIYGASNRHTGIPDRFSGSGSGTDFTLTISRLEPEDFAVYYCQQYHNAITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC